(19) United States
(12) Patent Application Publication
(10) Pub. No.: US 2017/0062735 A1
(43) Pub. Date: Mar. 2, 2017

(54) Benzotriphenylene Compound, Light-Emitting Element, Light-Emitting Device, Electronic Device, and Lighting Device

(71) Applicant: Semiconductor Energy Laboratory Co., Ltd., Kanagawa-ken (JP)

(72) Inventors: Kyoko TAKEDA, Atsugi (JP); Hiroshi KADOMA, Sagamihara (JP); Tsunenori SUZUKI, Yokohama (JP); Naoaki HASHIMOTO, Sagamihara (JP); Satoshi SEO, Sagamihara (JP)

(73) Assignee: Semiconductor Energy Laboratory Co., Ltd., Kanagawa-ken (JP)

(21) Appl. No.: 15/251,364

(22) Filed: Aug. 30, 2016

(30) Foreign Application Priority Data
Aug. 31, 2015 (JP) 2015-171076

(51) Int. Cl. H01L 51/00; C09K 11/02; C07D 209/80; C07D 209/86; C09K 11/06

(52) U.S. Cl. CPC H01L 51/0072; C07D 209/86; H01L 51/0055; C09K 11/06; C07D 209/80; C09K 11/02; H01L 51/5016

(57) ABSTRACT

A novel benzotriphenylene compound that can be suitably used as a host material in the case where a light-emitting substance is a fluorescent material. The benzotriphenylene compound is represented by General Formula (G1-1). In General Formula (G1-1), A represents a condensed ring. Each of R¹ to R⁹ and R¹¹ to R¹⁴ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Ar represents an arylene group having 6 to 13 carbon atoms.

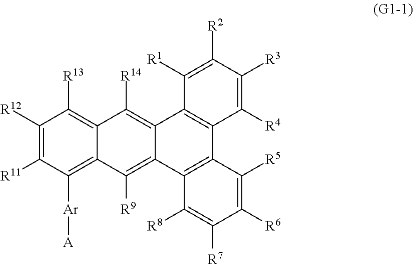

### BACKGROUND OF THE INVENTION

1. Field of the Invention

One embodiment of the present invention relates to a benzotriphenylene compound. One embodiment of the present invention relates to a light-emitting element in which a light-emitting layer capable of providing light emission by application of an electric field is provided between a pair of electrodes, and also relates to a light-emitting device, an electronic device, and a lighting device including the light-emitting element.

Note that one embodiment of the present invention is not limited to the above technical field. The technical field of one embodiment of the invention disclosed in this specification and the like relates to an object, a method, or a manufacturing method. In addition, one embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting device, a lighting device, a power storage device, a memory device, a method for driving any of them, and a method for manufacturing any of them.

2. Description of the Related Art

In recent years, research and development have been extensively conducted on light-emitting elements using electroluminescence (EL). In a basic structure of such a light-emitting element, a layer containing a light-emitting material (an EL layer) is interposed between a pair of electrodes. By application of a voltage to this element, light emission from the light-emitting material can be obtained.

Since the above light-emitting element is a self-luminous type, a display device using this light-emitting element has advantages such as high visibility, no necessity of a backlight, and low power consumption. Furthermore, such a light-emitting element also has advantages in that the element can be manufactured to be thin and lightweight, and has high response speed.

In the case of an organic EL element whose EL layer contains an organic compound as a light-emitting material and is provided between a pair of electrodes, application of a voltage between the pair of electrodes causes injection of electrons from a cathode and holes from an anode into the EL layer having a light-emitting property and thus a current flows. By recombination of the injected electrons and holes, the light-emitting organic compound is brought into an excited state to provide light emission.

Note that an excited state formed by an organic compound can be a singlet excited state (S*) or a triplet excited state (T*). Light emission from the singlet excited state is referred to as fluorescence, and light emission from the triplet excited state is referred to as phosphorescence. The formation ratio of S* to T* in the light-emitting element is statistically considered to be 1:3. In other words, a light-emitting element including a phosphorescent material has higher emission efficiency than a light-emitting element containing a fluorescent material. Therefore, light-emitting elements including phosphorescent materials capable of converting a triplet excited state into light emission have been actively developed in recent years.

Among light-emitting elements including phosphorescent materials, a light-emitting element that emits blue light in particular has yet been put into practical use because it is difficult to develop a stable compound having a high triplet excited energy level. For this reason, as a light-emitting element emitting blue light, a light-emitting element containing a more stable fluorescent material has been developed and a technique for increasing the emission efficiency of a light-emitting element containing a fluorescent material has been searched.

As an emission mechanism capable of converting part of a triplet excited state into light emission, triplet-triplet annihilation (TTA) that involves a plurality of triplet excitons is known. The term TTA refers to a process in which, when two triplet excitons approach each other, excited energy is transferred and spin angular momentum is exchanged to form a singlet exciton.

As compounds in which TTA occurs, anthracene compounds are known. Non-Patent Document 1 discloses that the use of an anthracene compound as a host material in a light-emitting element that emits blue light achieves an external quantum efficiency exceeding 10%. It also discloses that the proportion of the delayed fluorescence components due to TTA in the anthracene compound is approximately 10% of emissive components of the light-emitting element.

Furthermore, tetracene compounds are known as compounds in which delayed fluorescence components due to TTA account for a large proportion. Non-Patent Document 2 discloses that the delayed fluorescence components due to TTA in light emitted from a tetracene compound account for a larger proportion than that for an anthracene compound.

### REFERENCE

### [Non-Patent Document]

[Non-Patent Document 1] Tsunenori Suzuki and six others, Japanese Journal of Applied Physics, Vol. 53, 052102 (2014)
[Non-Patent Document 2] D. Y. Kondakov and three others, Journal of Applied Physics, Vol. 106, 124510 (2009)

### SUMMARY OF THE INVENTION

As reported in Non-Patent Document 1 or 2, although host materials for fluorescent materials have been developed, there is room for improvement in terms of emission efficiency, reliability, synthesis efficiency, cost, or the like, and further development is required for obtaining more excellent host materials for fluorescent materials.

In view of the above, an object of one embodiment of the present invention is to provide a novel benzotriphenylene compound that can be used in a light-emitting element as a host material in which a light-emitting substance of a light-emitting layer is dispersed. In particular, another object of one embodiment of the present invention is to provide a novel benzotriphenylene compound that can be suitably used as a host material in the case where the light-emitting substance is a fluorescent material. Another object of one embodiment of the present invention is to provide a novel benzotriphenylene compound that has a high electron-transport property and can be suitably used in an electron-transport layer in a light-emitting element. Another object of one embodiment of the present invention is to provide a novel light-emitting element.

Another object of one embodiment of the present invention is to provide a light-emitting element which is driven at a low voltage and has high current efficiency. Another object of one embodiment of the present invention is to provide a light-emitting element having a long lifetime. Another object of one embodiment of the present invention is to provide a light-emitting device, an electronic device, and a lighting device each having reduced power consumption. Another object of one embodiment of the present invention is to provide a novel light-emitting device, a novel electronic device, and a novel lighting device.

Note that the descriptions of these objects do not disturb the existence of other objects. In one embodiment of the present invention, there is no need to achieve all the objects. Other objects will be apparent from and can be derived from the description of the specification, the drawings, the claims, and the like.

One embodiment of the present invention is a benzotriphenylene compound represented by General Formula (G1-1).

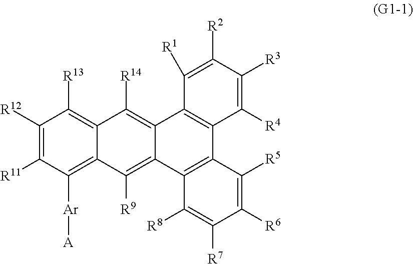

In General Formula (G1-1), A represents a condensed ring. Furthermore, each of R¹ to R⁹ and R¹¹ to R¹⁴ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Furthermore, Ar represents an arylene group having 6 to 13 carbon atoms.

In the above embodiment, the arylene group may include one or more substituents and the substituents may be bonded to each other to form a ring.

In the above embodiment, the condensed ring is preferably selected from a substituted or unsubstituted carbazolyl group, a substituted or unsubstituted naphthocarbazolyl group, a substituted or unsubstituted dibenzocarbazolyl group, and a substituted or unsubstituted benzocarbazolyl group. It is preferable that in the above embodiment, the condensed ring be a substituted or unsubstituted carbazolyl group and any one of the 2-position, the 3-position, and the 9-position of the carbazolyl group be bonded to Ar.

Note that the benzotriphenylene compound of one embodiment of the present invention can be rephrased as a benzo[b]triphenylene compound.

Another embodiment of the present invention is a benzotriphenylene compound represented by General Formula (G1-2).

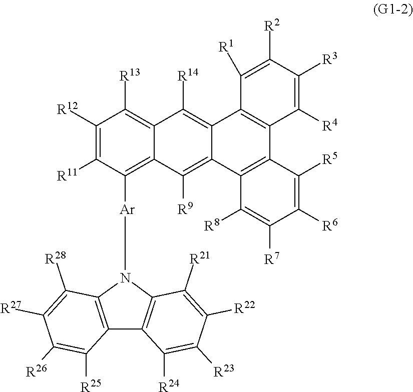

In General Formula (G1-2), each of R¹ to R⁹, R¹¹ to R¹⁴, and R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Furthermore, Ar represents an arylene group having 6 to 13 carbon atoms.

In the above embodiment, the arylene group may include one or more substituents and the substituents may be bonded to each other to form a ring.

Another embodiment of the present invention is a benzotriphenylene compound represented by General Formula (G2-1).

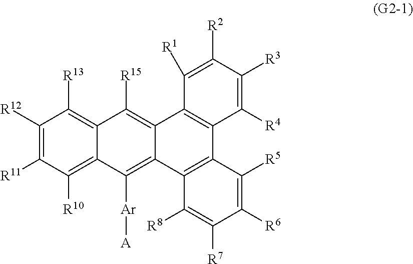

In General Formula (G2-1), A represents a condensed ring. Furthermore, each of R¹ to R⁸ and R¹⁰ to R¹³ independently represents any of hydrogen, an alkyl group having 1 to 6 carbon atoms, a cycloalkyl group having 3 to 6 carbon atoms, and a substituted or unsubstituted aryl group having 6 to 13 carbon atoms. R¹⁵ represents any of hydrogen, an alkyl group having 1 to 6 carbon atoms, and a cycloalkyl group having 3 to 6 carbon atoms. Furthermore, Ar represents an arylene group having 6 to 13 carbon atoms.

In the above embodiment, the arylene group may include one or more substituents and the substituents may be bonded to each other to form a ring.

In the above embodiment, the condensed ring is preferably selected from a substituted or unsubstituted carbazolyl group, a substituted or unsubstituted naphthocarbazolyl group, a substituted or unsubstituted dibenzocarbazolyl group, and a substituted or unsubstituted benzocarbazolyl group. It is preferable that in the above embodiment, the condensed ring be a substituted or unsubstituted carbazolyl group and any one of the 2-position, the 3-position, and the 9-position of the carbazolyl group be bonded to Ar.

Another embodiment of the present invention is a benzotriphenylene compound represented by General Formula (G2-2).

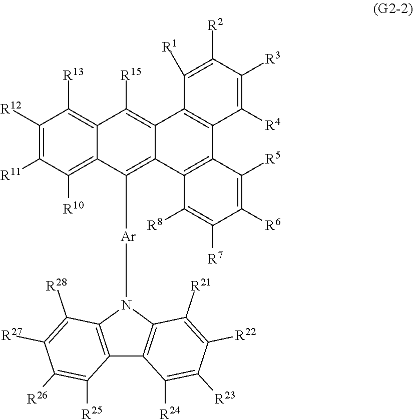

In General Formula (G2-2), each of R¹ to R⁸, R¹⁰ to R¹³, and R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms. R¹⁵ represents any of hydrogen, an alkyl group having 1 to 6 carbon atoms, and a cycloalkyl group having 3 to 6 carbon atoms. Furthermore, Ar represents an arylene group having 6 to 13 carbon atoms.

In the above embodiment, the arylene group may include one or more substituents and the substituents may be bonded to each other to form a ring.

In the above embodiments, Ar preferably represents a substituted or unsubstituted phenylene group or a substituted or unsubstituted biphenyldiyl group. In the above embodiments, Ar preferably represents a substituted or unsubstituted in-phenylene group.

Another embodiment of the present invention is a benzotriphenylene compound represented by Structural Formula (100) or Structural Formula (200).

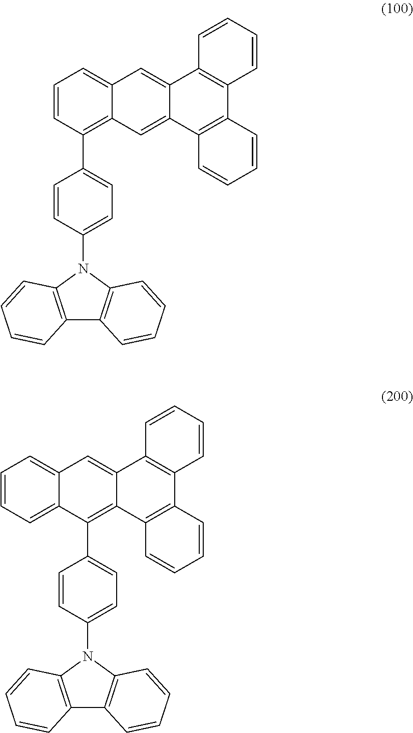 [SMILES: c1cc(-c2ccc(-n3c4ccccc4c4ccccc43)cc2)c2cc3c4ccccc4c4ccccc4c3cc2c1 ; c1ccc2c(-c3ccc(-n4c5ccccc5c5ccccc54)cc3)c3c4ccccc4c4ccccc4c3cc2c1]

Another embodiment of the present invention is a light-emitting element that includes a pair of electrodes and an EL layer between the pair of electrodes. The EL layer includes the benzotriphenylene compound of any one of the above embodiments.

In the above embodiment, it is preferable that the EL layer further include a fluorescent material.

In the above embodiment, the fluorescent material preferably has a peak of an emission spectrum in a blue wavelength range. In the above embodiments, the fluorescent material preferably exhibits delayed fluorescence.

Another embodiment of the present invention is a light-emitting device that includes any of the above light-emitting elements and a color filter. Another embodiment of the present invention is an electronic device that includes the above light-emitting device and a housing or a touch sensor. Another embodiment of the present invention is a lighting device that includes any one of the above light-emitting elements and a housing.

The category of one embodiment of the present invention includes not only the light-emitting device including the light-emitting element but also an electronic device including the light-emitting device. Therefore, the light-emitting device in this specification refers to an image display device or a light source (e.g., a lighting device). In addition, the light-emitting device includes all the following modules: a module in which a connector, such as a flexible printed circuit (FPC) or a tape carrier package (TCP), is attached to a light-emitting device; a module in which a printed wiring board is provided at the end of a TCP; and a module in which an integrated circuit (IC) is directly mounted on a light-emitting element by a chip-on-glass (COG) method.

One embodiment of the present invention makes it possible to provide a novel benzotriphenylene compound that can be used in a light-emitting element as a host material in which a light-emitting substance of a light-emitting layer is dispersed. One embodiment of the present invention makes it possible to provide a novel benzotriphenylene compound that can be suitably used as a host material in the case where the light-emitting substance is a fluorescent material. One embodiment of the present invention makes it possible to provide a novel benzotriphenylene compound that has a high electron-transport property and can be suitably used in an electron-transport layer in a light-emitting element. One embodiment of the present invention makes it possible to provide a novel light-emitting element.

One embodiment of the present invention makes it possible to provide a light-emitting element which is driven at a low voltage and has high current efficiency. One embodiment of the present invention makes it possible to provide a light-emitting element having a long lifetime. One embodiment of the present invention makes it possible to provide a light-emitting device, an electronic device, and a lighting device each having reduced power consumption. One embodiment of the present invention makes it possible to provide a novel light-emitting device, a novel electronic device, and a novel lighting device.

Note that the description of these effects does not disturb the existence of other effects. One embodiment of the present invention does not necessarily achieve all the effects listed above. Other effects will be apparent from and can be derived from the description of the specification, the drawings, the claims, and the like.

### BRIEF DESCRIPTION OF THE DRAWINGS

FIGS. 1A and 1B are cross-sectional views illustrating a light-emitting element of one embodiment of the present invention and FIG. 1C is a schematic view illustrating the correlation of energy levels.

FIG. 2 is a cross-sectional view illustrating a light-emitting element of one embodiment of the present invention.

FIGS. 3A and 3B are cross-sectional views each illustrating a light-emitting element of one embodiment of the present invention.

FIG. 4A is a cross-sectional view illustrating a light-emitting element of one embodiment of the present invention and FIG. 4B is a schematic view illustrating the correlation of energy levels.

FIG. 5A is a cross-sectional view illustrating a light-emitting element of one embodiment of the present invention and FIG. 5B is a schematic view illustrating the correlation of energy levels.

FIGS. 6A and 6B are a block diagram and a circuit diagram illustrating a display device.

FIGS. 7A and 7B are each a circuit diagram illustrating a pixel circuit of a display device.

FIGS. 8A and 8B are each a circuit diagram illustrating a pixel circuit of a display device.

FIGS. 9A and 9B are perspective views illustrating an example of a touch panel.

FIGS. 10A to 10C are cross-sectional views illustrating examples of a display panel and a touch sensor.

FIGS. 11A and 11B are cross-sectional views each illustrating an example of a touch panel.

FIGS. 12A and 12B are a block diagram and a timing chart of a touch sensor.

FIG. 13 is a circuit diagram of a touch sensor.

FIG. 14 is a perspective view illustrating a display module.

FIGS. 15A to 15G illustrate electronic devices.

FIGS. 16A and 16B are perspective views illustrating a display device.

FIGS. 17A to 17C are a perspective view and cross-sectional views illustrating a light-emitting device.

FIGS. 18A to 18D are cross-sectional views each illustrating a light-emitting device.

FIGS. 19A and 19B illustrate a lighting device and an electronic device.

FIGS. 20A and 20B show NMR charts of 9-[4-(benzo[b]triphenylen-9-yl)phenyl]-9H-carbazole (abbreviation: 9CzPBTp).

FIG. 21 shows an emission spectrum of 9-[4-(benzo[b]triphenylen-9-yl)phenyl]-9H-carbazole (abbreviation: 9CzPBTp).

FIG. 22 shows an absorption spectrum of 9-[4-(benzo[b]triphenylen-9-yl)phenyl]-9H-carbazole (abbreviation: 9CzPBTp).

FIG. 23 shows an emission spectrum of 9-[4-(benzo[b]triphenylen-9-yl)phenyl]-9H-carbazole (abbreviation: 9CzPBTp).

FIG. 24 shows an absorption spectrum of 9-[4-(benzo[b]triphenylen-9-yl)phenyl]-9H-carbazole (abbreviation: 9CzPBTp).

FIGS. 25A and 25B show NMR charts of 9-[4-(benzo[b]triphenylen-10-yl)phenyl]-9H-carbazole (abbreviation: 10CzPBTp).

FIG. 26 shows an emission spectrum of 9-[4-(benzo[b]triphenylen-10-yl)phenyl]-9H-carbazole (abbreviation: 10CzPBTp).

FIG. 27 shows an absorption spectrum of 9-[4-(benzo[b]triphenylen-10-yl)phenyl]-9H-carbazole (abbreviation: 10CzPBTp).

FIG. 28 shows an emission spectrum of 9-[4-(benzo[b]triphenylen-10-yl)phenyl]-9H-carbazole (abbreviation: 10CzPBTp).

FIG. 29 shows an absorption spectrum of 9-[4-(benzo[b]triphenylen-10-yl)phenyl]-9H-carbazole (abbreviation: 10CzPBTp).

FIGS. 30A and 30B show MIR charts of 7-[4-(benzo[b]triphenylen-10-yl)phenyl]-7H-dibenzo[c,g]carbazole (abbreviation: 10cgDBCzPBTp).

FIG. 31 shows an emission spectrum of 7-[4-(benzo[b]triphenylen-10-yl)phenyl]-7H-dibenzo[c,g]carbazole (abbreviation: 10cgDBCzPBTp).

FIG. 32 shows an absorption spectrum of 7-[4-(benzo[b]triphenylen-10-yl)phenyl]-7H-dibenzo[c,g]carbazole (abbreviation: 10cgDBCzPBTp).

FIG. 33 shows an emission spectrum of 7-[4-(benzo[b]triphenylen-10-yl)phenyl]-7H-dibenzo[c,g]carbazole (abbreviation: 10cgDBCzPBTp).

FIG. 34 shows an absorption spectrum of 7-[4-(benzo [b]triphenylen-10-yl)phenyl]-7H-dibenzo[c,g]carbazole (abbreviation: 10cgDBCzPBTp).

FIGS. 35A and 35B are cross-sectional views each illustrating a light-emitting element in Example.

FIG. 36 is a graph showing luminance vs. current density characteristics of light-emitting elements of embodiments of the present invention.

FIG. 37 is a graph showing luminance vs. voltage characteristics of light-emitting elements of embodiments of the present invention.

FIG. 38 is a graph showing current efficiency vs. luminance characteristics of light-emitting elements of embodiments of the present invention.

FIG. 39 is a graph showing current vs. voltage characteristics of light-emitting elements of embodiments of the present invention.

FIGS. 40A and 40B are graphs showing fluorescence lifetime characteristics of light-emitting elements of embodiments of the present invention.

FIG. 41 is a graph showing luminance vs. current density characteristics of light-emitting elements of embodiments of the present invention.

FIG. 42 is a graph showing luminance vs. voltage characteristics of light-emitting elements of embodiments of the present invention.

FIG. 43 is a graph showing current efficiency vs. luminance characteristics of light-emitting elements of embodiments of the present invention.

FIG. 44 is a graph showing current vs. voltage characteristics of light-emitting elements of embodiments of the present invention.

FIG. 45 is a graph showing reliability test results of light-emitting elements of embodiments of the present invention.

### DETAILED DESCRIPTION OF THE INVENTION

Embodiments of the present invention will be explained in detail below with reference to the drawings. However, the present invention is not limited to description to be given below, and it is to be easily understood that modes and details thereof can be variously modified without departing from the purpose and the scope of the present invention. Accordingly, the present invention should not be interpreted as being limited to the content of the embodiments below.

Note that the position, the size, the range, or the like of each structure illustrated in drawings and the like is not accurately represented in some cases for easy understanding. Therefore, the disclosed invention is not necessarily limited to the position, the size, the range, or the like disclosed in the drawings and the like.

Ordinal numbers such as “first” and “second” in this specification and the like are used for convenience and do not denote the order of steps or the stacking order of layers. Therefore, for example, description can be made even when “first” is replaced with “second” or “third”, as appropriate. In addition, the ordinal numbers in this specification and the like are not necessarily the same as those which specify one embodiment of the present invention.

In the description of modes of the present invention in this specification and the like with reference to the drawings, the same components in different diagrams are commonly denoted by the same reference numeral in some cases.

In this specification and the like, the terms “film” and “layer” can be interchanged with each other. For example, the term “conductive layer” can be changed into the term “conductive film” in some cases. Also, the term “insulating film” can be changed into the term “insulating layer” in some cases.

In this specification and the like, a singlet excited state (S*) means a singlet state having excited energy. Among singlet excited states, an excited state having the lowest energy is referred to as the lowest singlet excited state. Furthermore, a singlet excited energy level means an energy level in a singlet excited state. Among singlet excited energy levels, the lowest excited energy level is referred to as the lowest singlet excited energy (S₁) level.

In this specification and the like, a triplet excited state (T*) means a triplet state having excited energy. Among triplet excited states, an excited state having the lowest energy is referred to as the lowest triplet excited state. Furthermore, a triplet excited energy level means an energy level in a triplet excited state. Among triplet excited energy levels, the lowest excited energy level is referred to as the lowest triplet excited energy (T₁) level.

In this specification and the like, a fluorescent material refers to a material that emits light in the visible light region when the singlet excited state relaxes to the ground state. A phosphorescent material refers to a material that emits light in the visible light region at room temperature when the triplet excited state relaxes to the ground state. That is, a phosphorescent material refers to a material that can convert triplet excited energy into visible light.

In this specification and the like, the blue wavelength range refers to a range in which the wavelength is greater than or equal to 400 nm and less than or equal to 550 nm, and the blue light emission refers to light emission with at least one emission spectrum peak in the range.

### Embodiment 1

In this embodiment, a benzotriphenylene compound that is an organic compound of one embodiment of the present invention will be described.

<1-1. Benzotriphenylene compounds represented by General Formulae (G1-1) and (G1-2)>

One embodiment of the present invention is a benzotriphenylene compound represented by General Formula (G1-1).

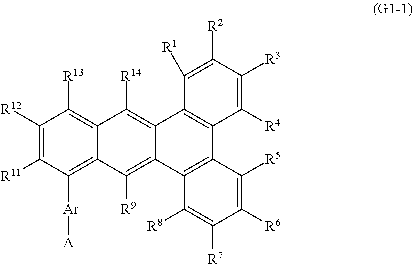

In General Formula (G1-1), A represents a condensed ring. Furthermore, each of R¹ to R⁹ and R¹¹ to R¹⁴ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Furthermore, Ar represents an arylene group having 6 to 13 carbon atoms. The arylene group may include one or more substituents and the substituents may be bonded to each other to form a ring.

The condensed ring in General Formula (G1-1) is preferably selected from a substituted or unsubstituted carbazolyl group, a substituted or unsubstituted naphthocarbazolyl group, a substituted or unsubstituted dibenzocarbazolyl group, and a substituted or unsubstituted benzocarbazolyl group. It is particularly preferable that the condensed ring be a substituted or unsubstituted carbazolyl group and any one of the 2-position, the 3-position, and the 9-position of the carbazolyl group be bonded to the Ar. A typical example is a benzotriphenylene compound represented by General Formula (G1-2). Note that the benzotriphenylene compound represented by General Formula (G1-2) is one embodiment of the present invention.

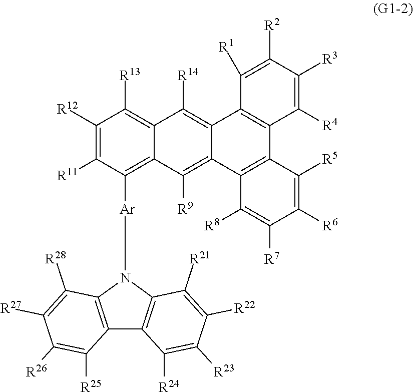

In General Formula (G1-2), each of R¹ to R⁹, R¹¹ to R¹⁴, and R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Furthermore, Ar represents an arylene group having 6 to 13 carbon atoms. The arylene group may include one or more substituents and the substituents may be bonded to each other to form a ring.

### <1-2. Benzotriphenylene Compounds Represented by General Formulae (G2-1) and (G2-2)>

Another embodiment of the present invention is a benzotriphenylene compound represented by General Formula (G2-1).

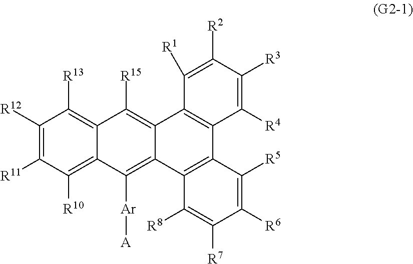

In General Formula (G2-1), A represents a condensed ring. Furthermore, each of R¹ to R⁸ and R¹° to R¹³ independently represents any of hydrogen, an alkyl group having 1 to 6 carbon atoms, a cycloalkyl group having 3 to 6 carbon atoms, and a substituted or unsubstituted aryl group having 6 to 13 carbon atoms. R¹⁵ represents any of hydrogen, an alkyl group having 1 to 6 carbon atoms, and a cycloalkyl group having 3 to 6 carbon atoms. Furthermore, Ar represents an arylene group having 6 to 13 carbon atoms. The arylene group may include one or more substituents and the substituents may be bonded to each other to form a ring.

The condensed ring in General Formula (G2-1) is preferably selected from a substituted or unsubstituted carbazolyl group, a substituted or unsubstituted naphthocarbazolyl group, a substituted or unsubstituted dibenzocarbazolyl group, and a substituted or unsubstituted benzocarbazolyl group. It is particularly preferable that the condensed ring be a substituted or unsubstituted carbazolyl group and any one of the 2-position, the 3-position, and the 9-position of the carbazolyl group be bonded to the Ar. A typical example is a benzotriphenylene compound represented by General Formula (G2-2). Note that the benzotriphenylene compound represented by General Formula (G2-2) is one embodiment of the present invention.

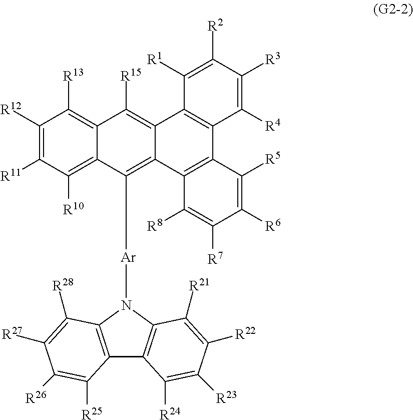

In General Formula (G2-2), each of R¹ to R⁸, R¹⁰ to R¹³, and R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms. R¹⁵ represents any of hydrogen, an alkyl group having 1 to 6 carbon atoms, and a cycloalkyl group having 3 to 6 carbon atoms.

Furthermore, Ar represents an arylene group having 6 to 13 carbon atoms. The arylene group may include one or more substituents and the substituents may be bonded to each other to form a ring.

In General Formulae (G1-1), (G1-2), (G2-1), and (G2-2), it is preferable that Ar represent a substituted or unsubstituted phenylene group or a substituted or unsubstituted biphenyldiyl group. It is particularly preferable that Ar represent a substituted or unsubstituted phenylene group or a substituted or unsubstituted m-phenylene group.

<1-3. Specific examples of R¹ to R¹⁴ and R²¹ to R²⁸>

As specific examples of structures of R¹ to R¹⁴ and R²¹ to R²⁸ in General Formulae (G1-1), (G1-2), (G2-1), and (G2-2), groups represented by Structural Formulae (R-1) to (R-23) can be given.

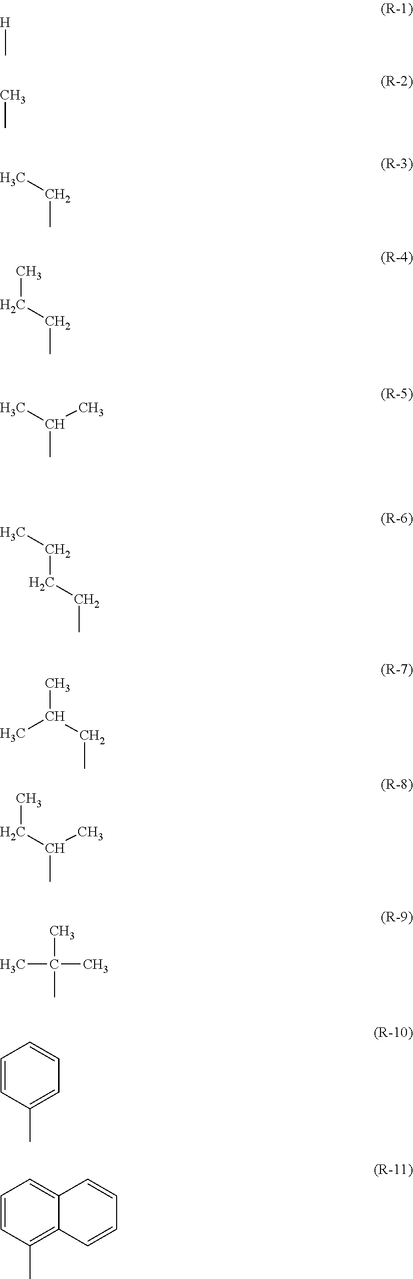

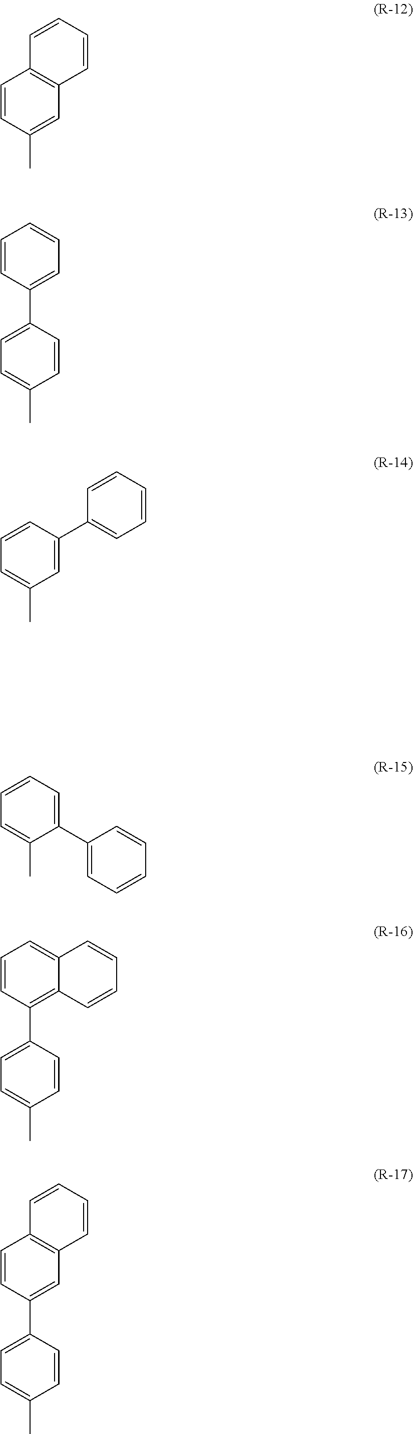

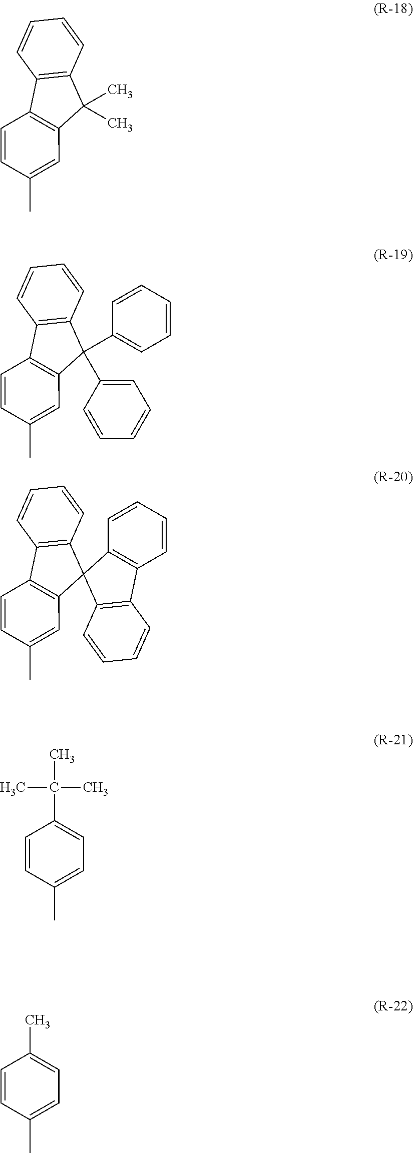

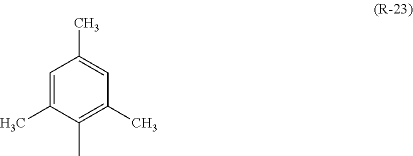

### <1-4. Specific Examples of Ar>

As specific examples of structures of Ar in General Formulae (G1-1), (G1-2), (G2-1), and (G2-2), groups represented by Structural Formulae (Ar-1) to (Ar-15) can be given.

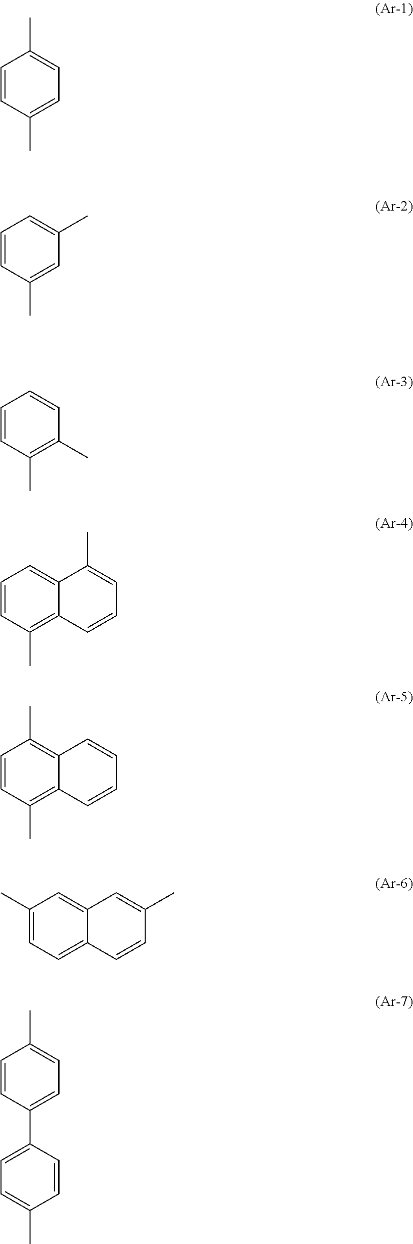

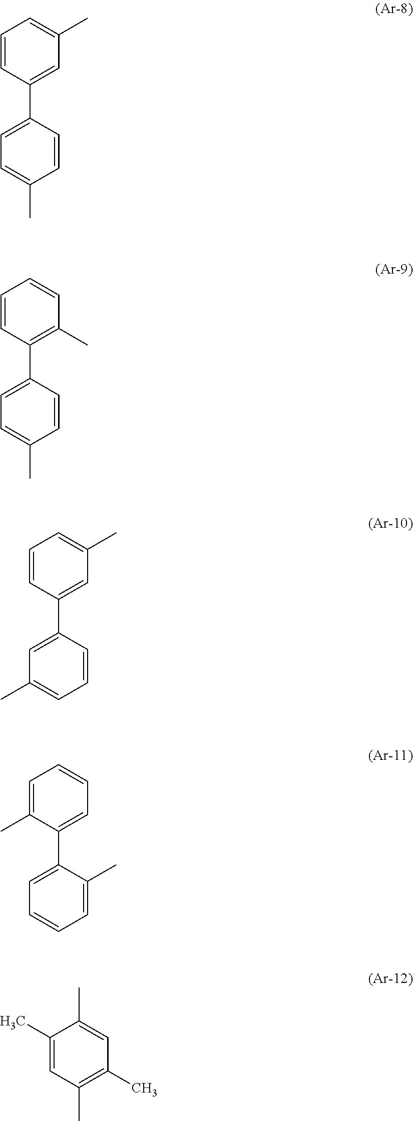

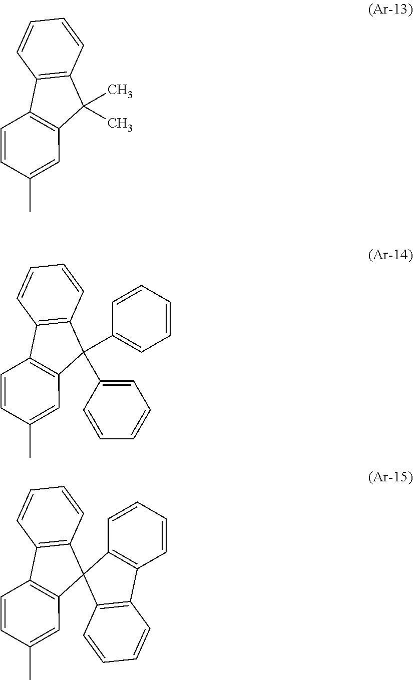

### <1-5. Specific Examples of Benzotriphenylene Compound>

As specific examples of the benzotriphenylene compounds represented by General Formulae (G1-1) and (G1-2), benzotriphenylene compounds represented by Structural Formulae (100) to (160) can be given.

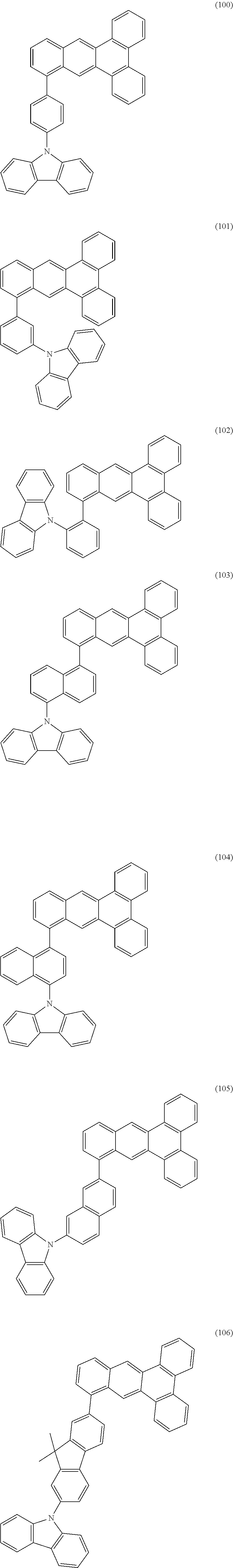

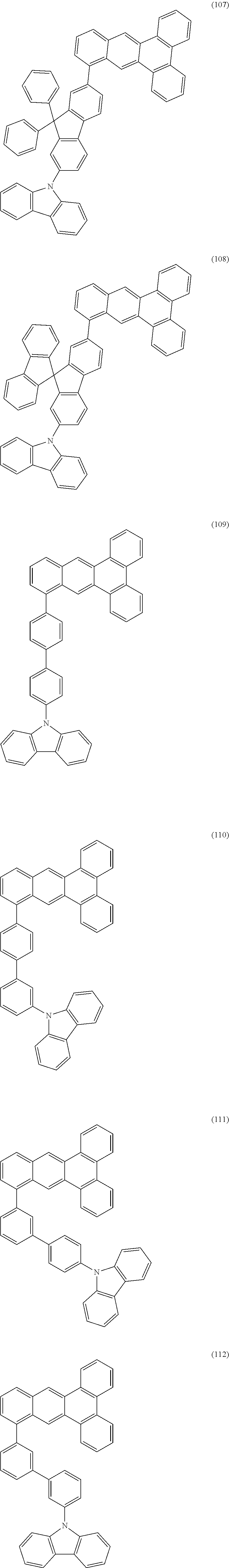

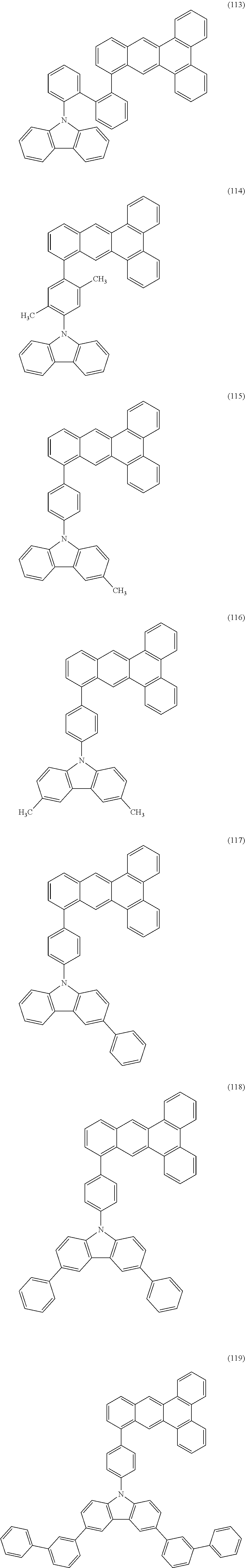

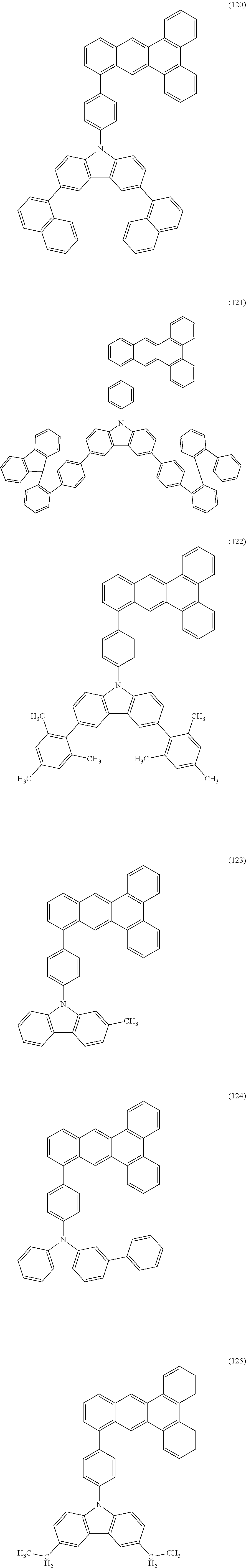

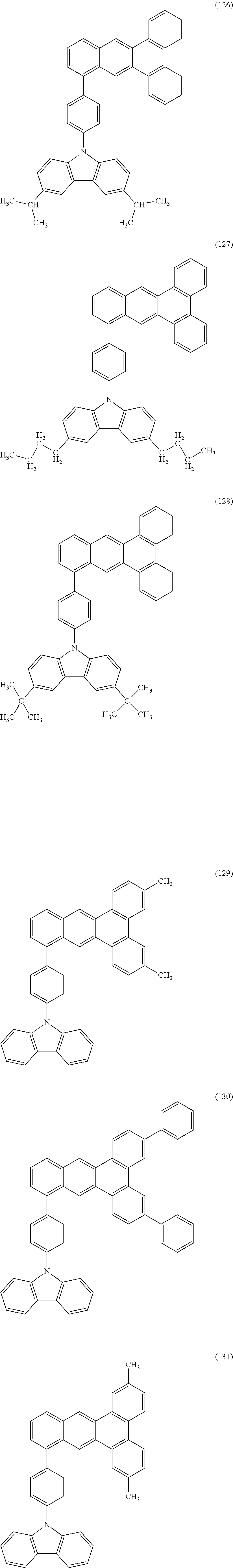

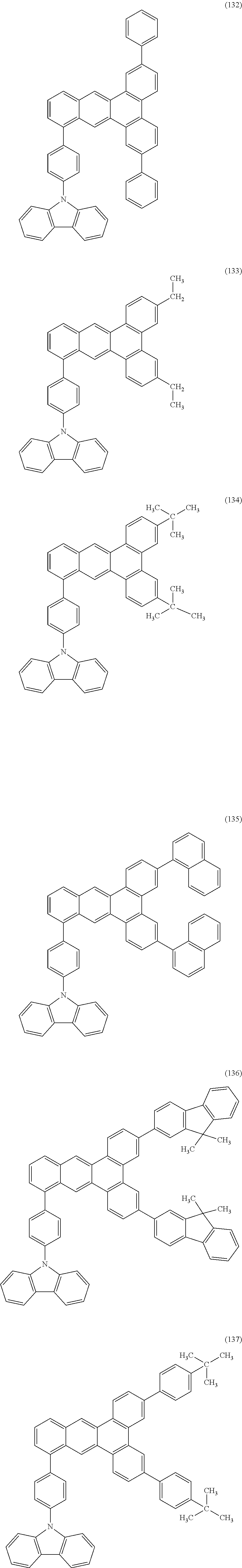

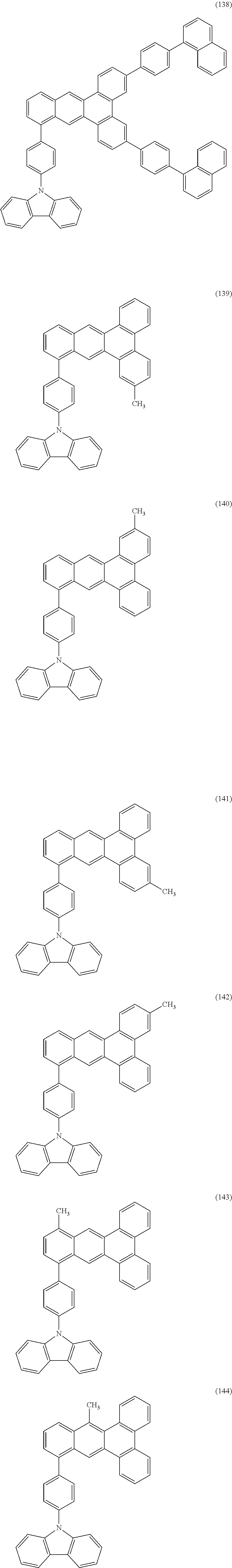

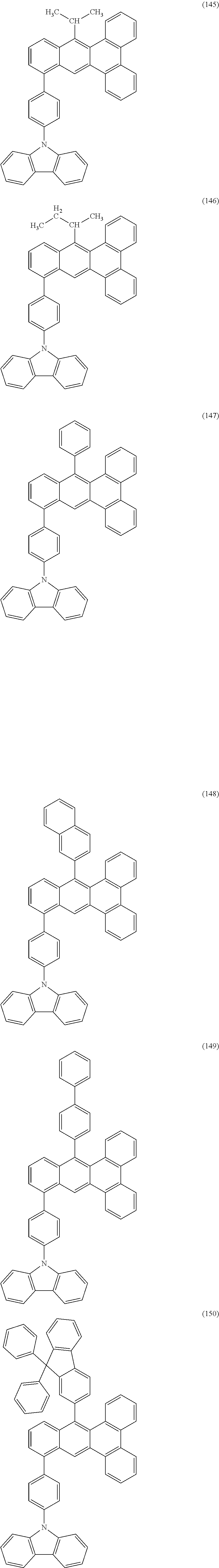

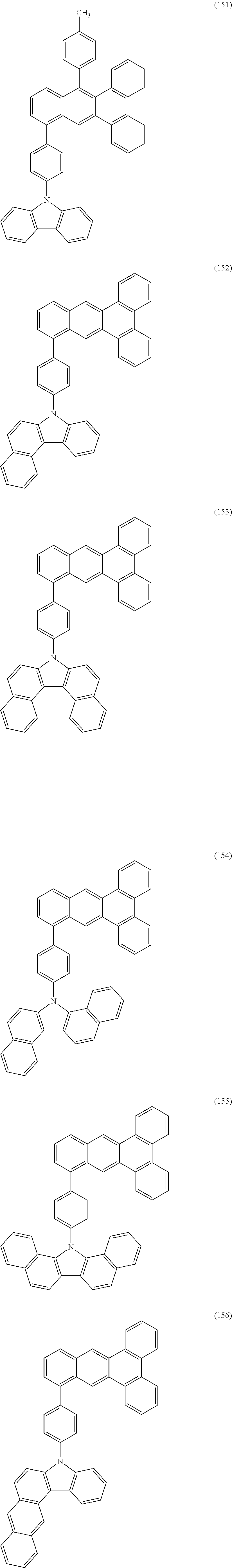

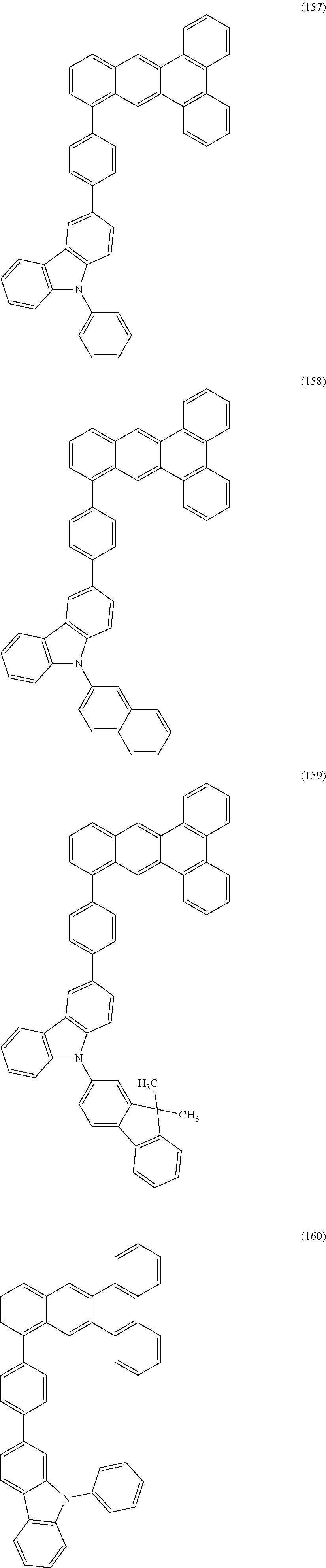

As specific examples of the benzotriphenylene compounds represented by General Formulae (G2-1) and (G2-2), benzotriphenylene compounds represented by Structural Formulae (200) to (253) can be given.

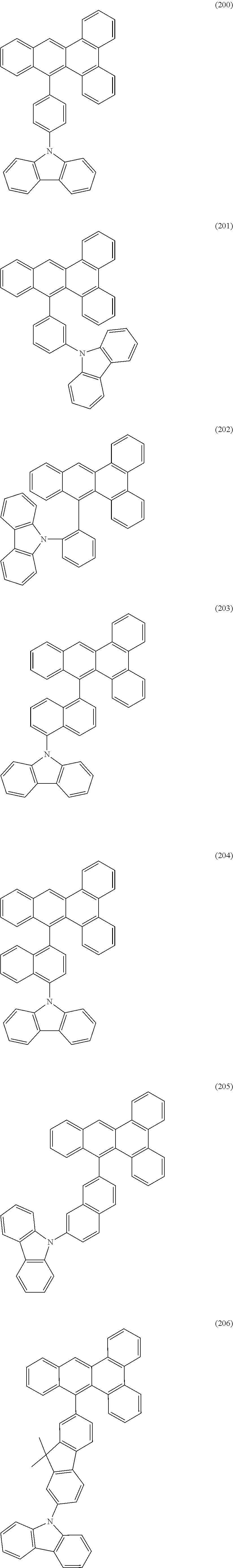

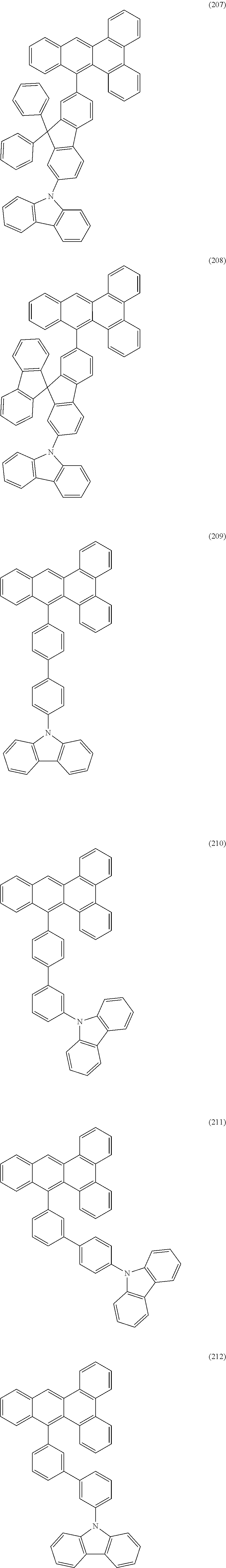

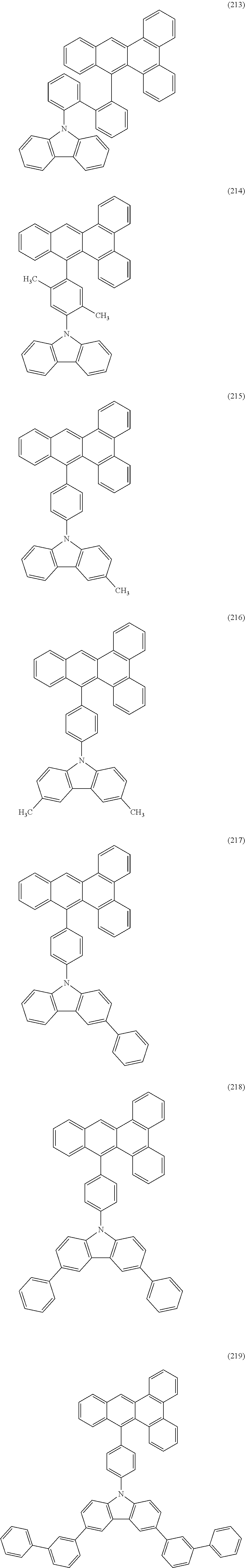

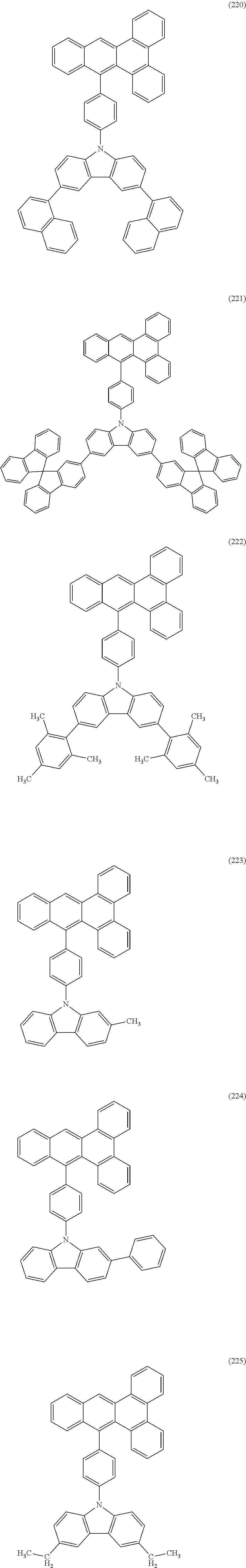

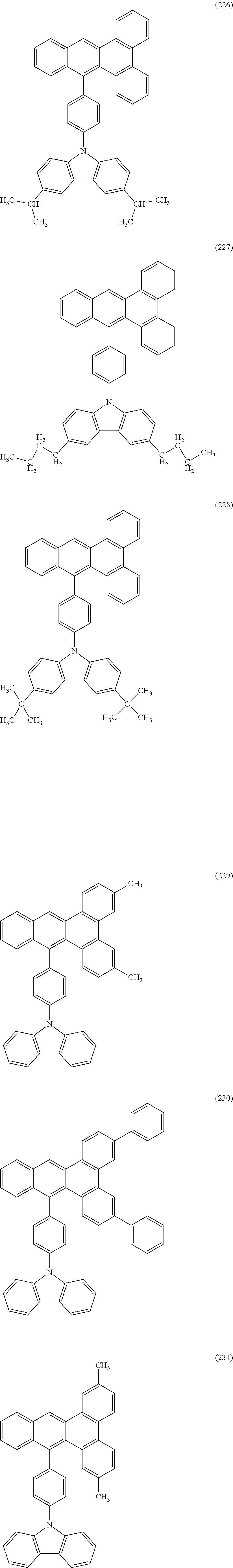

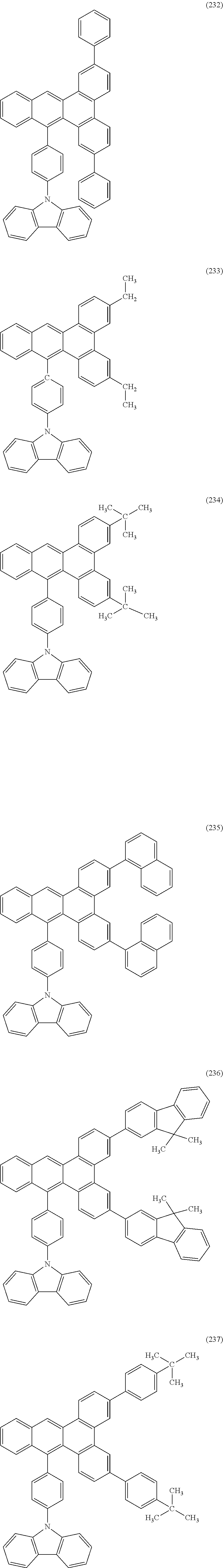

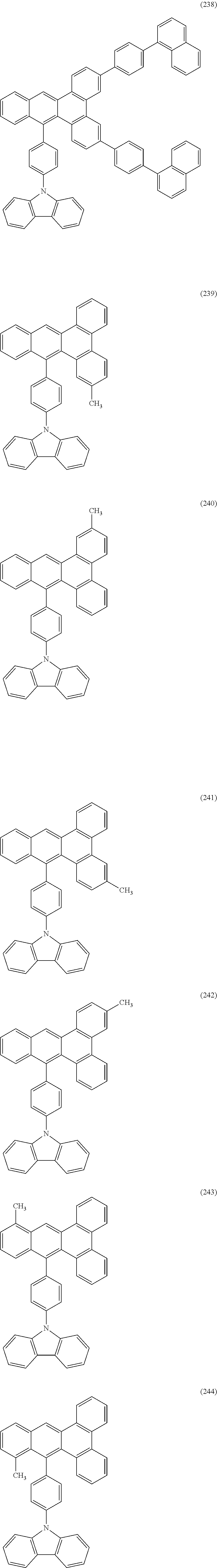

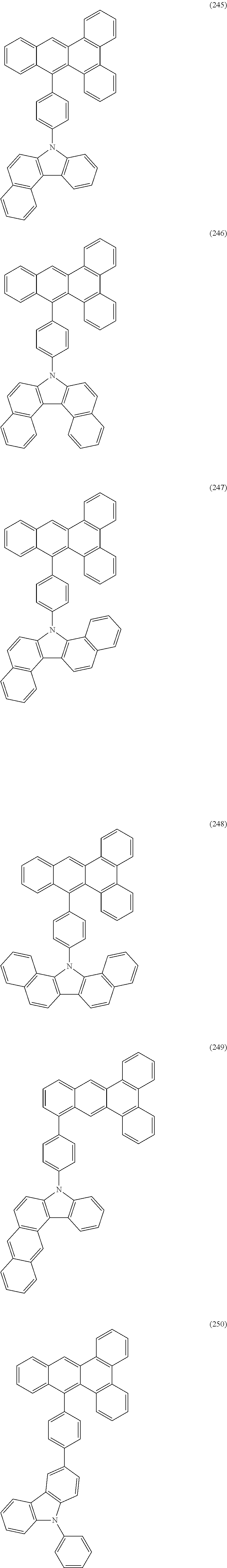

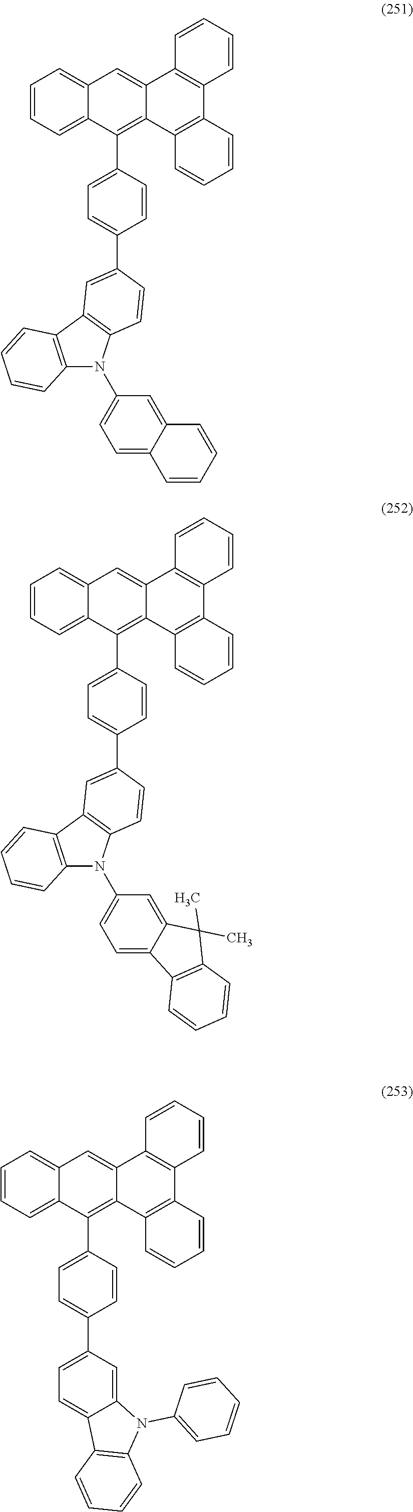

The benzotriphenylene compound of one embodiment of the present invention is preferably represented by Structural Formula (100) or (200), in which case the synthesis can be performed easily. Note that the benzotriphenylene compound of one embodiment of the present invention is not limited to the benzotriphenylene compounds represented by Structural Formulae (100) to (160) and Structural Formulae (200) to (253).

### <1-6. Method for Synthesizing Benzotriphenylene Compound Represented by General Formula (G1-1)>

Next, an example of a method for synthesizing the benzotriphenylene compound represented by General Formula (G1-1) will be described. A variety of reactions can be applied to the method for synthesizing the benzotriphenylene compound represented by General Formula (G1-1). For example, the benzotriphenylene compound represented by General Formula (G1-1) can be synthesized by a synthesis reaction shown below. As shown in Synthesis Scheme (a-1), a halide of a benzo[b]triphenylene derivative or a benzo[b]triphenylene derivative having a triflate group as a substituent (compound 1) is coupled with an organoboron compound or a boronic acid compound of a carbazole derivative or a condensed polycyclic carbazole derivative (compound 2) by a Suzuki-Miyaura coupling reaction, whereby the target compound (G1-1) can be obtained.

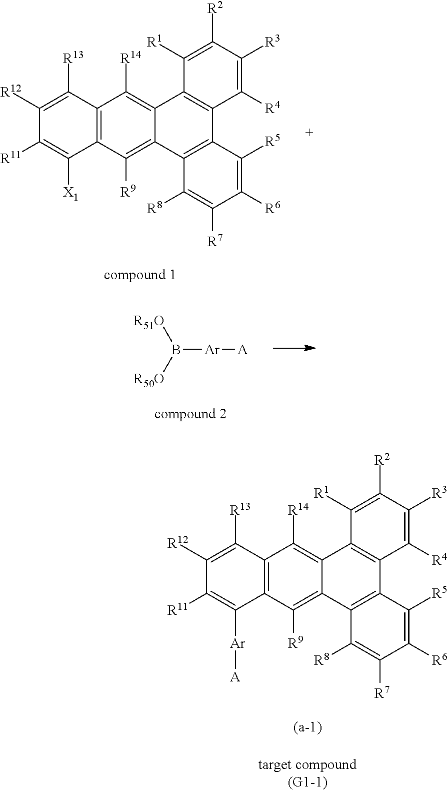

Note that in Synthesis Scheme (a-1), A represents a substituted or unsubstituted carbazolyl group, a substituted or unsubstituted naphthocarbazolyl group, a substituted or unsubstituted dibenzocarbazolyl group, or a substituted or unsubstituted benzocarbazolyl group, and each of R¹ to R⁹ and R¹¹ to R¹⁴ independently represents any of hydrogen, an alkyl group having 1 to 6 carbon atoms, a cycloalkyl group having 3 to 6 carbon atoms, and a substituted or unsubstituted aryl group having 6 to 13 carbon atoms. Furthermore, Ar represents an arylene group having 6 to 13 carbon atoms. The arylene group may include one or more substituents and the substituents may be bonded to each other to form a ring.

In Synthesis Scheme (a-1), each of R⁵⁰ and R⁵¹ independently represents any of hydrogen and an alkyl group having 1 to 6 carbon atoms, and R⁵° and R⁵¹ may be bonded to each other to form a ring. Furthermore, X¹ represents a halogen or a triflate.

### <1-7. Method for Synthesizing Benzotriphenylene Compound Represented by General Formula (G2-1)>

Next, an example of a method for synthesizing the benzotriphenylene compound represented by General Formula (G2-1) will be described. A variety of reactions can be applied to the method for synthesizing the benzotriphenylene compound represented by General Formula (G2-1). For example, the benzotriphenylene compound represented by General Formula (G2-1) can be synthesized by a synthesis reaction shown below. As shown in Synthesis Scheme (b-1), a halide of a benzo[b]triphenylene derivative or a benzo[b]triphenylene derivative having a triflate group as a substituent (compound 3) is coupled with an organoboron compound or a boronic acid compound of a carbazole derivative or a condensed polycyclic carbazole derivative (compound 2) by a Suzuki-Miyaura coupling reaction, whereby the target compound (G2-1) can be obtained.

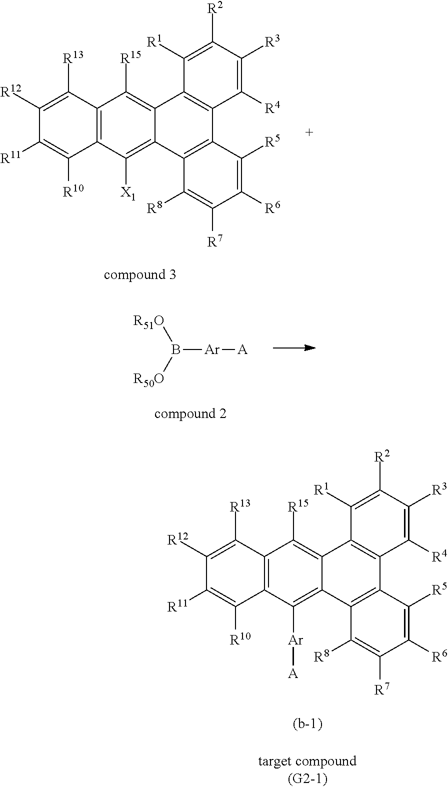

In Synthesis Scheme (b-1), A represents a substituted or unsubstituted carbazolyl group, a substituted or unsubstituted naphthocarbazolyl group, a substituted or unsubstituted dibenzocarbazolyl group, or a substituted or unsubstituted benzocarbazolyl group, and each of R¹ to R⁸ and R¹⁰ to R¹³ independently represents any of hydrogen, an alkyl group having 1 to 6 carbon atoms, a cycloalkyl group having 3 to 6 carbon atoms, and a substituted or unsubstituted aryl group having 6 to 13 carbon atoms. R¹⁵ represents any of hydrogen, an alkyl group having 1 to 6 carbon atoms, and a cycloalkyl group having 3 to 6 carbon atoms. Furthermore, Ar represents an arylene group having 6 to 13 carbon atoms. The arylene group may include one or more substituents and the substituents may be bonded to each other to form a ring.

In Synthesis Scheme (b-1), R⁵⁰ and R⁵¹ may be bonded to each other to form a ring. Furthermore, X¹ represents a halogen or a triflate group, and the halogen is preferably iodine or bromine.

Examples of a palladium catalyst that can be used in Synthesis Schemes (a-1) and (b-1) include, but are not limited to, palladium(II) acetate, tetrakis(triphenylphosphine)palladium(0), and bis(triphenylphosphine)palladium(II) dichloride. Examples of a ligand of the palladium catalyst that can be used in Synthesis Schemes (a-1) and (b-1) include, but are not limited to, tri(ortho-tolyl)phosphine, triphenylphosphine, and tricyclohexylphosphine.

Examples of a base that can be used in Synthesis Schemes (a-1) and (b-1) include, but not limited to, an organic base such as sodium tert-butoxide and an inorganic base such as potassium carbonate or sodium carbonate.

Examples of the solvent that can be used in Synthesis Schemes (a-1) and (b-1) include, but are not limited to, a mixed solvent of toluene and water; a mixed solvent of toluene, an alcohol such as ethanol, and water; a mixed solvent of xylene and water; a mixed solvent of xylene, an alcohol such as ethanol, and water; a mixed solvent of benzene and water; a mixed solvent of benzene, an alcohol such as ethanol, and water; and a mixed solvent of water and an ether such as ethylene glycol dimethyl ether. In particular, a mixed solvent of toluene and water, a mixed solvent of toluene, ethanol, and water, or a mixed solvent of water and an ether such as ethylene glycol dimethyl ether is preferred.

The Suzuki-Miyaura coupling reaction shown in Synthesis Schemes (a-1) and (b-1) may be replaced with a cross coupling reaction using an organoaluminum compound, an organozirconium compound, an organozinc compound, an organotin compound, or the like as well as the organoboron compound or boronic acid compound represented by the compound 2. However, the coupling reaction is not limited thereto.

In the Suzuki-Miyaura coupling reaction shown in each of Synthesis Schemes (a-1) and (b-1), an organoboron compound or a boronic acid compound of a benzo[b]triphenylene derivative may be coupled with a halide of a carbazole derivative, a halide of a condensed polycyclic carbazole derivative, a carbazole derivative having a triflate group as a substituent, or a condensed polycyclic carbazole derivative having a triflate group as a substituent.

In the above manner, the organic compound of one embodiment of the present invention can be synthesized. Note that the synthesis method of any of the organic compounds of embodiments of the present invention is not limited to the synthesis methods above.

Since the benzotriphenylene compound of one embodiment of the present invention has a high S₁ level and a wide energy gap (Eg) between the HOMO level and the LUMO level, high current efficiency can be obtained by using the benzotriphenylene compound in a light-emitting element as a host material of a light-emitting layer in which a light-emitting substance is dispersed. In particular, the benzotriphenylene compound is suitably used as a host material in which a fluorescent material is dispersed. Furthermore, since the benzotriphenylene compound of one embodiment of the present invention has a high electron-transport property, the benzotriphenylene compound can be suitably used as a material for an electron-transport layer in a light-emitting element.

By using the benzotriphenylene compound of one embodiment of the present invention, a light-emitting element with low driving voltage and high current efficiency can be obtained. Furthermore, by the use of this light-emitting element, a light-emitting device, an electronic device, and a lighting device each having reduced power consumption can be obtained.

Note that the structure described in this embodiment can be used in appropriate combination with any of the other embodiments and examples.

### Embodiment 2

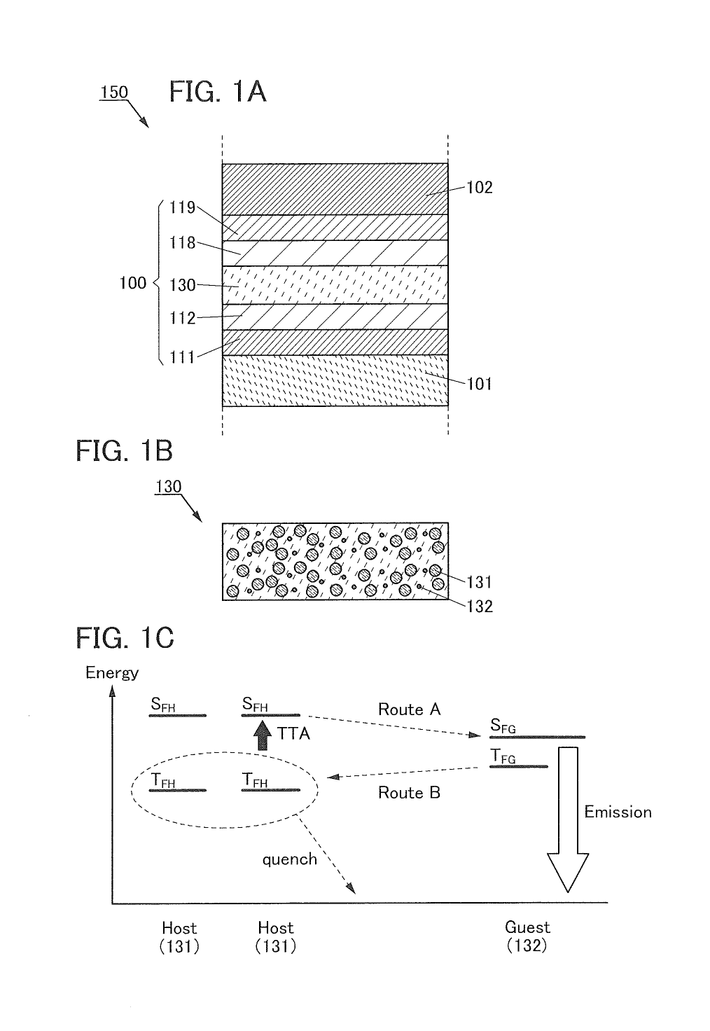

In this embodiment, a structure of a light-emitting element that includes the benzotriphenylene compound described in Embodiment 1 will be described with reference to FIGS. 1A to 1C.

### <2-1. Structure 1 of Light-Emitting Element>

First, a structure of a light-emitting element of one embodiment of the present invention is described below with reference to FIGS. 1A to 1C.

FIG. 1A is a schematic cross-sectional view of a light-emitting element 150 of one embodiment of the present invention.

The light-emitting element 150 includes an EL layer 100 between a pair of electrodes (a first electrode 101 and a second electrode 102). The EL layer 100 includes at least a light-emitting layer 130. Note that in this embodiment, although description is given assuming that the first electrode 101 and the second electrode 102 of the pair of electrodes serve as an anode and a cathode, respectively, the first electrode 101 and the second electrode 102 may serve as a cathode and an anode, respectively, for the structure of the light-emitting element 150.

The EL layer 100 illustrated in FIG. 1A includes functional layers in addition to the light-emitting layer 130. The functional layers include a hole-injection layer 111, a hole-transport layer 112, an electron-transport layer 118, and an electron-injection layer 119. Note that the structure of the EL layer 100 is not limited to the structure illustrated in FIG. 1A, and a structure may be employed in which at least one selected from the hole-injection layer 111, the hole-transport layer 112, the electron-transport layer 118, and the electron-injection layer 119 is included. Alternatively, the EL layer 100 may include a functional layer which is has a function of lowering a hole injection barrier or an electron injection barrier, improving a hole-transport property or an electron-transport property, inhibiting a hole-transport property or an electron-transport property, or suppressing a quenching phenomenon by an electrode, for example.

FIG. 1B is a schematic cross-sectional view of an example of the light-emitting layer 130 in FIG. 1A. The light-emitting layer 130 in FIG. 1B includes a host material 131 and a guest material 132.

The host material 131 preferably has a function of converting triplet excited energy into singlet excited energy by causing TTA, in which case the triplet excited energy generated in the light-emitting layer 130 can be partly converted into singlet excited energy by TTA in the host material 131. The singlet excited energy generated by TTA can be transferred to the guest material 132 and extracted as fluorescence.

In order to fulfill the above function, the lowest singlet excited energy (S₁) level of the host material 131 is preferably higher than the S₁ level of the guest material 132. In addition, the lowest triplet excited energy (T₁) level of the host material 131 is preferably lower than the T₁ level of the guest material 132.

The host material 131 may be composed of a single material or a plurality of materials. The guest material 132 may be a light-emitting organic compound, and the light-emitting organic compound is preferably a substance capable of emitting fluorescence (hereinafter also referred to as a fluorescent material). A structure in which a fluorescent material is used as the guest material 132 will be described below. Note that the guest material 132 may be read as the fluorescent material. Note that the light-emitting layer 130 may have a structure without the guest material 132, i.e., a structure consisting only of the host material 131. In that case, fluorescence is extracted from the host material 131 in the light-emitting layer 130.

### <2-2. Emission Mechanism of Light-Emitting Element>

First, an emission mechanism of the light-emitting element 150 is described below.

In the light-emitting element 150 of one embodiment of the present invention, voltage application between a pair of electrodes (the first and second electrodes 101 and 102) causes electrons and holes to be injected from the cathode and the anode, respectively, into the EL layer 100 and current flows. By recombination of the injected electrons and holes, excited states are formed in the light-emitting layer 130 of the EL layer 100. The ratio of singlet excited states to triplet excited states in the excited states formed by the carrier recombination (hereinafter referred to as exciton generation probability) is 1:3 according to the statistically obtained probability.

Note that singlet excitons are formed in the EL layer 100 and light emission from the guest material 132 can be obtained through the following two processes:

(α) direct formation process; and

(β) TTA process.

### <2-3. (α) Direct Formation Process>

The case where carriers (electrons and holes) recombine in the light-emitting layer 130 included in the EL layer 100 to form a singlet exciton is first described.

First, when the carriers recombine in the host material 131, an excited state of the host material 131 is formed (a singlet excited state or a triplet excited state). At this time, in the case where the excited state of the host material 131 is a singlet excited state, singlet excited energy transfers from the S₁ level of the host material 131 to the S₁ level of the guest material 132, thereby forming the singlet excited state of the guest material 132. Note that the case where the excited state of the host material 131 is a triplet excited state is described in later in (β) TTA process.

When the carriers recombine in the guest material 132, an excited state of the guest material 132 is formed (a singlet excited state or a triplet excited state). When the excited state of the guest material 132 is a singlet excited state at this time and the fluorescent quantum efficiency of the guest material 132 is high, light is efficiently emitted from the singlet excited state of the guest material 132.

In the case where the formed excited state of the guest material 132 is a triplet excited state, the triplet excited state of the guest material 132 is thermally deactivated and does not contribute to light emission. However, if the T₁ level of the host material 131 is lower than the T₁ level of the guest material 132, the triplet excited energy of the guest material 132 can be transferred from the T₁ level of the guest material 132 to the T₁ level of the host material 131. In this case, the triplet excited energy can be converted into singlet excited energy by (β) TTA process described later.

In the case where the T₁ level of the host material 131 is higher than the T₁ level of the guest material 132, if the concentration of the guest material 132 with respect to the host material 131 is low, the probability of carrier recombination in the guest material 132 can be reduced. In addition, the probability of energy transfer from the T₁ level of the host material 131 to the T₁ level of the guest material 132 can be reduced. Specifically, the concentration of the guest material 132 with respect to the host material 131 is preferably 5 wt % or lower.

<2-4. (β) TTA process>

The case where a singlet exciton is formed from triplet excitons formed in the carrier recombination process in the light-emitting layer 130 is described.

Here, the case where the T₁ level of the host material 131 is lower than the T₁ level of the guest material 132 is described. The correlation of energy levels in this case is schematically shown in FIG. 1C. What terms and signs in FIG. 1C represent are listed below. Note that the T₁ level of the host material 131 may be higher than the T₁ level of the guest material 132.

Host: the host material 131

Guest: the guest material 132 (fluorescent material)

S_{FH}: the S₁ level of the host material 131

T_{FH}: the T₁ level of the host material 131

S_{FG}: the S₁ level of the guest material 132 (fluorescent material)

T_{FG}: the T₁ level of the guest material 132 (fluorescent material)

Carriers recombine in the host material 131 and the host material 131 is brought into an excited state. At this time, in the case where the excited state of the host material 131 is a triplet excited state and the generated triplet excitons approach each other, a reaction in which part of their triplet excited energy is converted into singlet excited energy and the triplet excitons are converted into singlet excitons having energy of the S₁ level (S_{FH}) of the host material 131 might be caused (see TTA in FIG. 1C). This is represented by General Formula (G11) or (G12) below.

³H+³H→¹H*+¹H (G11)

³H+³H→³H*+¹H (G12)

General Formula (G11) represents a reaction in the host material 131 in which a singlet exciton (¹H*) is fonned from two triplet excitons (³H) with a total spin quantum number of 0. General Formula (G12) represents a reaction in the host material 131 in which an electronically or oscillatorily excited triplet exciton (³H*) is formed from two triplet excitons (³H) with a total spin quantum number of 1 (atomic unit). In General Formulae (G11) and (G12), ¹H represents the singlet ground state of the host material 131.

Although the reactions in General Formulae (G11) and (G12) occur at the same probability, there are three times as many pairs of triplet excitons with a total spin quantum number of 1 (atomic unit) as pairs of triplet excitons with a total spin quantum number of 0. In other words, when an exciton is formed from two triplet excitons, the singlet-triplet exciton formation ratio is 1:3 according to the statistically obtained probability. In the case where the density of the triplet excitons in the light-emitting layer 130 is sufficiently high (10⁻¹² cm⁻³ or more), only the reaction of two triplet excitons approaching each other can be considered whereas deactivation of a single triplet exciton is ignored.

Thus, from General Formula (G11) and General Formula (G12), one singlet exciton (¹H*) and three triplet excitons (³H*) which are electronically or oscillatorily excited are formed from eight triplet excitons (³H). This is represented by General Formula (G13).

8³H→¹H*+3³H*+4¹H (G13)

The electronically or oscillatorily excited triplet excitons (³H*), which are formed as in General Formula (G13), become triplet excitons (³H) by relaxation and then repeat the reaction in General Formula (G13) again with other triplet excitons. Hence, in General Formula (G13), if all the triplet excitons (³H) are converted into singlet excitons (¹H*), five triplet excitons (³H) form one singlet exciton (¹H*).

The total formation probability of singlet excitons can be increased, when combined with the formation probability of singlet excitons directly formed by recombination of carriers injected from a pair of electrodes, i.e., 25%, to 40% at the maximum by TTA (General Formula (G14)). That is, TTA can increase the probability of singlet exciton formation by 15% from 25%, which is the conventional value, to 40%.

5¹H*+15³H→5¹H*+(3¹H*+12¹H) (G14)

In the singlet excited state of the host material 131 which is formed by TTA, energy is transferred from the S₁ level (S_{FH}) of the host material 131 to the S₁ level (S_{FG}) of the guest material 132, which is lower than S_{FH} (see Route A in FIG. 1C). Then, the guest material 132 brought into a singlet excited state emits fluorescence.

In the case where carriers recombine in the guest material 132 and a formed excited state is a triplet excited state, if the T₁ level (T_{FH}) of the host material 131 is lower than the T₁ level (T_{FG}) of the guest material, T_{FG} is not deactivated and energy is transferred to T_{FH} (see Route B in FIG. 1C) to contribute to TTA.

In the case where the T₁ level (T_{FG}) of the guest material 132 is lower than the T₁ level (T_{FH}) of the host material 131, the concentration of the guest material 132 with respect to the host material 131 is preferably low. Specifically, the concentration of the guest material 132 with respect to the host material 131 is preferably 5 wt % or lower, in which case, the probability of carrier recombination in the guest material 132 can be reduced. In addition, the probability of energy transfer from the T₁ level (T_{FH}) of the host material 131 to the T₁ level (T_{FG}) of the guest material 132 can be reduced.

As described above, triplet excitons formed in the light-emitting layer 130 can be converted into singlet excitons by TTA, so that light emitted from the guest material 132 can be efficiently obtained.

Next, details of components of the light-emitting element 150 in FIG. 1A are described.

### [Pair of Electrodes]

The first electrode 101 and the second electrode 102 have functions of injecting holes and electrons into the light-emitting layer 130. The first and second electrodes 101 and 102 can be formed using a metal, an alloy, or a conductive compound, or a mixture or a stack thereof, for example. A typical example of the metal is aluminum; besides, a transition metal such as silver, tungsten, chromium, molybdenum, copper, or titanium, an alkali metal such as lithium or cesium, or a Group 2 metal such as calcium or magnesium can be used. As the transition metal, a rare earth metal may be used. An alloy containing any of the above metals can be used as the alloy, and an Ag-Mg alloy and an Al—Li alloy can be given as examples. As examples of the conductive compound, metal oxides such as an In-Sn oxide (also referred to as ITO) and an In—Sn—Si oxide (also referred to as ITSO) can be given. It is also possible to use an inorganic carbon-based material such as graphene as the conductive compound. As described above, the first electrode 101 and/or the second electrode 102 may be formed by stacking two or more of these materials.

Light emitted from the light-emitting layer 130 is extracted through the first electrode 101 and/or the second electrode 102. Therefore, at least one of the first and second electrodes 101 and 102 transmits visible light. In the case where the electrode through which light is extracted is formed using a material with low light transmittance, such as a metal or an alloy, the first electrode 101 and/or the second electrode 102 are/is formed to be thin enough to transmit visible light (e.g., a thickness of 1 nm to 10 nm).

### [Hole-Injection Layer]

The hole-injection layer 111 has a function of reducing a barrier for hole injection from one of the pair of electrodes (the first electrode 101 or the second electrode 102) to promote hole injection and is formed using a transition metal oxide, a phthalocyanine derivative, or an aromatic amine, for example. As the transition metal oxide, molybdenum oxide, vanadium oxide, ruthenium oxide, tungsten oxide, manganese oxide, or the like can be given. As the phthalocyanine derivative, phthalocyanine, metal phthalocyanine, or the like can be given. As the aromatic amine, a benzidine derivative, a phenylenediamine derivative, or the like can be given. It is also possible to use a high molecular compound such as polythiophene or polyaniline; a typical example thereof is poly(ethylenedioxythiophene)/poly(styrenesulfonic acid), which is self-doped polythiophene.

As the hole-injection layer 111, a mixed layer containing a hole-transport material and a material having a property of accepting electrons from the hole-transport material can also be used. Alternatively, a stack of a layer containing a material having an electron accepting property and a layer containing a hole-transport material may also be used. In a steady state or in the presence of an electric field, electric charge can be transferred between these materials. As examples of the material having an electron-accepting property, organic acceptors such as a quinodimethane derivative, a chloranil derivative, and a hexaazatriphenylene derivative can be given. Alternatively, a transition metal oxide such as an oxide of a metal from Group 4 to Group 8 can also be used. Specifically, vanadium oxide, niobium oxide, tantalum oxide, chromium oxide, molybdenum oxide, tungsten oxide, manganese oxide, rhenium oxide, or the like can be used. In particular, molybdenum oxide is preferable because it is stable in the air, has a low hygroscopic property, and is easily handled.

A material having a property of transporting more holes than electrons can be used as the hole-transport material, and a material having a hole mobility of 1×10⁻⁶ cm²/Vs or higher is preferable. Specifically, an aromatic amine, a carbazole derivative, an aromatic hydrocarbon, a stilbene derivative, or the like can be used. Furthermore, the hole-transport material may be a high molecular compound.

Examples of the material having a high hole-transport property are N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), and the like.

Specific examples of the carbazole derivative are 3-[N-(9-phenylcarbazol-3-yl)-N-phenylamino]-9-phenylcarbazole (abbreviation: PCzPCAl), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), and the like.

Other examples of the carbazole derivative are 4,4′-di(N-carbazolyl)biphenyl (abbreviation: CBP), 1,3,5-tris [4-(N-carbazolyl)phenyl]benzene (abbreviation: TCPB), 9-[4-(10-phenyl-9-anthryl)phenyl]-9H-carbazole (abbreviation: CzPA), 1,4-bis[4-(N-carbazolyl)phenyl]-2,3,5,6-tetraphenylbenzene, and the like.

Examples of the aromatic hydrocarbon are 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis [2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, and the like. Other examples are pentacene, coronene, and the like. The aromatic hydrocarbon having a hole mobility of 1×10⁻⁶ cm²Ns or more and having 14 to 42 carbon atoms is particularly preferable.

The aromatic hydrocarbon may have a vinyl skeleton. Examples of the aromatic hydrocarbon having a vinyl group are 4,4′-bis(2,2-diphenylvinyl)biphenyl (abbreviation: DPVBi), 9,10-bis [4-(2,2-diphenylvinyl)phenyl]anthracene (abbreviation: DPVPA), and the like.

Other examples are high molecular compounds such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N-bis(phenyl)benzidine] (abbreviation: poly-TPD).

### [Hole-Transport Layer]

The hole-transport layer 112 is a layer containing a hole-transport material and can be formed using any of the materials given as examples of the material for the hole-injection layer 111. In order that the hole-transport layer 112 can have a function of transporting holes injected into the hole-injection layer 111 to the light-emitting layer 130, the highest occupied molecular orbital (HOMO) level of the hole-transport layer 112 is preferably equal or close to the HOMO level of the hole-injection layer 111.

In addition to the hole-transport materials given as the material for the hole-injection layer 111, examples of the substance having a high hole-transport property are aromatic amine compounds such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), and the like. The substances described here are mainly substances having a hole mobility of 1×10⁻⁶ cm²/Vs or higher. Note that any substance other than the above substances may be used as long as the hole-transport property is higher than the electron-transport property. The layer including a substance having a high hole-transport property is not limited to a single layer, and two or more layers containing the aforementioned substances may be stacked.

### [Light-Emitting Layer]

The host material 131 in the light-emitting layer 130 is preferably an organic compound in which delayed fluorescence components due to triplet-triplet annihilation (TTA) account for a large proportion of emitted light, or typically, an organic compound in which delayed fluorescence components due to TTA account for 10% or more. It is particularly favorable to use the benzotriphenylene compound of one embodiment of the present invention described in Embodiment 1 as the host material 131. In the light-emitting layer 130, the host material 131 may be composed of one kind of compound or a plurality of compounds.

In the light-emitting layer 130, as the guest material 132, any of the following materials can be used.

The examples include 5,6-bis[4-(10-phenyl-9-anthryl)phenyl]-2,2′-bipyridine (abbreviation: PAP2BPy),

5,6-bis[4′-(10-phenyl-9-anthryl)biphenyl-4-yl]-2,2′-bipyridine (abbreviation: PAPP2BPy),

N,N′-diphenyl-N,N′-bis[4-(9-phenyl-9H-fluoren-9-yephenyl]pyrene-1,6-diamine (abbreviation: 1,6FLPAPrn),

N,N′-bis(3-methylphenyl)-N,N′-bis[3-(9-phenyl-9H-fluoren-9-yl)phenyl]pyrene-1,6-diamine (abbreviation: 1,6mMemFLPAPm),

N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA),

N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA),

N,N′-(2-tert-butylanthracene-9,10-diyldi-4,1-phenylene)bis[N,N,N′-triphenyl-1,4-phenylenediamine] (abbreviation: DPABPA),

N,9-diphenyl-N-[4-(9,10-diphenyl-2-anthryl)phenyl]-9H-carbazol-3-amine (abbreviation: 2PCAPPA),

N-[4-(9,10-diphenyl-2-anthryl)phenyl]-N,N′,N′-triphenyl-1,4-phenylenediamine (abbreviation: 2DPAPPA),

N,N,N′,N′,N″,N″,N′″,N′″-octaphenyldibenzo[gp]chrysene-2,7,10,15-tetraamine (abbreviation: DBC1), coumarin 30,

N-(9,10-diphenyl-2-anthryl)-N,9-diphenyl-9H-carbazol-3-amine (abbreviation: 2PCAPA),

N-[9,10-bis(1,1′-biphenyl-2-yl)-2-anthryl]-N,9-diphenyl-9H-carbazol-3-amine (abbreviation: 2PCABPhA),

N-(9,10-diphenyl-2-anthryl)-N,N′,N′-triphenyl-1,4-phenylenediamine (abbreviation: 2DPAPA),

N-[9,10-bis(1,1′-biphenyl-2-yl)-2-anthiyl]-N,N′,N′-triphenyl-1,4-phenylenediamine (abbreviation: 2DPABPhA),

9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 6, coumarin 545T, N,N′-diphenylquinacridone (abbreviation: DPQd), rubrene, 5,12-bis(1,1′-biphenyl-4-yl)-6,11-diphenyltetracene (abbreviation: BPT),

2-(2-{2-[4-(dimethylamino)phenyl]ethenyl}-6-methyl-4H-pyran-4-ylidene)propanedinitrile (abbreviation: DCM1),

2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD),

2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI),

2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB),

2-(2,6-bis{2-[4-(dimethylamino)phenyl]ethenyl}-4H-pyran-4-ylidene)propanedinitrile (abbreviation: BisDCM),

2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), and

5,10,15,20-tetraphenylbisbenzo[5,6]indeno[1,2,3-cd:1′,2′,3′-lm]perylene.

The light-emitting layer 130 may include a material other than the host material 131 and the guest material 132.

Although there is no particular limitation on a material that can be used in the light-emitting layer 130, any of the following materials can be used, for example: metal complexes such as tris(8-quinolinolato)aluminum(Ill) (abbreviation: Alq), tris(4-methyl-8-quinolinolato)aluminum(III) (abbreviation: Almq₃), bis(10-hydroxybenzo[h]quinolinato)beryllium(ll) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)-tris(1-phenyl-1H-benzimidazole) (abbreviation: TPBI), bathophenanthroline (abbreviation: BPhen), bathocuproine (abbreviation: BCP), and 9-[4-(5-phenyl-1,3,4-oxadiazol-2-yl)phenyl]-9H-carbazole (abbreviation: CO11); and aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), and 4,4′-bis[N-(spiro-9,9′-bifluoren-2-yl)-N-phenylamino]biphenyl (abbreviation: BSPB). In addition, condensed polycyclic aromatic compounds such as anthracene derivatives, phenanthrene derivatives, pyrene derivatives, chrysene derivatives, and dibenzo[g,p]chrysene derivatives can be used. Specific examples thereof include 9,10-diphenylanthracene (abbreviation: DPAnth), N,N-diphenyl-9-[4-(10-phenyl-9-anthryl)phenyl]-9H-carbazol-3-amine (abbreviation: CzAlPA), 4-(10-phenyl-9-anthryl)triphenylamine (abbreviation: DPhPA), 4-(9H-carbazol-9-yl)-4′-(10-phenyl-9-anthryl)triphenylamine (abbreviation: YGAPA),

N,9-diphenyl-N-[4-(10-phenyl-9-anthryl)phenyl]-9H-carbazol-3-amine (abbreviation: PCAPA),

N,9-diphenyl-N-{4-[4-(10-phenyl-9-anthryfiphenyl]phenyl}-9H-carbazol-3 -amine (abbreviation: PCAPBA),

N,9 diphenyl N(9,10-diphenyl-2-anthryl)-9H-carbazol-3-amine (abbreviation: 2PCAPA), 6,12-dimethoxy-5,11-diphenylchrysene, N,N,N′,N′,N″, N″,N′″,N′″-octaphenyldibenzo[g,p]chrysene-2,7,10,15-tetraamine (abbreviation: DBC1), 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tent-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyediphenanthrene (abbreviation: DPNS2), and 1,1′,1″-(benzene-1,3,5-triyl)tripyrene (abbreviation: TPB3). Two or more of the benzotriphenylene compounds of embodiments of the present invention may be included. One or more substances having a wider energy gap than the guest material 132 may be selected from the above substances.

The light-emitting layer 130 can have a structure in which two or more layers are stacked. For example, in the case where the light-emitting layer 130 is formed by stacking a first light-emitting layer and a second light-emitting layer in this order from the hole-transport layer side, the first light-emitting layer is formed using a substance having a hole-transport property as the host material and the second light-emitting layer is formed using a substance having an electron-transport property as the host material. Also in such a case, at least one light-emitting layer preferably includes the benzotriphenylene compound of one embodiment of the present invention.

### [Electron-Transport Layer]

The electron-transport layer 118 has a function of transporting, to the light-emitting layer 130, electrons injected from the other of the pair of electrodes (the first electrode 101 or the second electrode 102) through the electron-injection layer 119. A material having a property of transporting more electrons than holes can be used as an electron-transport material, and a material having an electron mobility of 1×10⁻⁶ cm²/Vs or higher is preferable. Specific examples include a metal complex having a quinoline ligand, a benzoquinoline ligand, an oxazole ligand, or a thiazole ligand; an oxadiazole derivative; a triazole derivative; a phenanthroline derivative; a pyridine derivative; a bipyridine derivative; and a pyrimidine derivative.

For example, the electron-transport layer is formed using a metal complex having a quinoline skeleton or a benzoquinoline skeleton, such as tris(8-quinolinolato)aluminum (abbreviation: Alq), tris(4-methyl-8-quinolinolato)aluminum (abbreviation: Almq₃), bis(10-hydroxybenzo[h]quinolinato)beryllium (abbreviation: BeBq₂), or bis(2-methyl-8-quinolinolato)(4-phenylphenolato)aluminum (abbreviation: BAlq), or the like. A metal complex having an oxazole-based or thiazole-based ligand, such as bis[2-(2-hydroxyphenyl)benzoxazolato]zinc (abbreviation: Zn(BOX)₂) or bis[2-(2-hydroxyphenyl)benzothiazolato]zinc (abbreviation: Zn(BTZ)₂), or the like can also be used. Other than the metal complexes, 2-(4-biphenylyl)-5-(4-tent-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), bathophenanthroline (abbreviation: BPhen), bathocuproine (abbreviation: BCP), or the like can also be used. The substances given here are mainly ones having an electron mobility of 10⁻⁶ cm²/Vs or higher.

The benzotriphenylene compound of one embodiment of the present invention can also be favorably used for the electron-transport layer 118. Note that any substance other than the above substances may be used for the electron-transport layer as long as the substance has an electron-transport property higher than a hole-transport property. Furthermore, the electron-transport layer 118 is not limited to a single layer and may be a stack of two or more layers including any of the above substances.

Between the electron-transport layer 118 and the light-emitting layer 130, a layer that controls transport of electron carriers may be provided. This is a layer formed by addition of a small amount of a substance having a high electron-trapping property to the aforementioned material having a high electron-transport property, and the layer is capable of adjusting carrier balance by retarding transport of electron carriers. Such a structure is very effective in preventing a problem (such as a reduction in element lifetime) caused when electrons pass through the light-emitting layer.

### [Electron-Injection Layer]

The electron-injection layer 119 has a function of reducing a barrier for electron injection from the second electrode 102 to promote electron injection and can be formed using a Group 1 metal or a Group 2 metal, or an oxide, a halide, or a carbonate of any of the metals, for example. Alternatively, a composite material containing an electron-transport material (described above) and a material having a property of donating electrons to the electron-transport material can also be used. As the material having an electron-donating property, a Group 1 metal, a Group 2 metal, an oxide of any of the metals, or the like can be given.

### [Substrate]

The light-emitting element 150 is fabricated over a substrate of glass, plastic, or the like. As the way of stacking layers over the substrate, layers may be sequentially stacked from the first electrode 101 side or sequentially stacked from the second electrode 102 side.

Note that, for example, glass, quartz, plastic, or the like can be used for the substrate over which the light-emitting element 150 can be formed. Alternatively, a flexible substrate can be used. A flexible substrate is a substrate that can be bent (is flexible); examples of the flexible substrate include a plastic substrate made of a polycarbonate, a polyarylate, or a polyethersulfone, and the like. A film (made of polypropylene, a polyester, poly(vinyl fluoride), poly(vinyl chloride), or the like), an inorganic film formed by evaporation, or the like can be used. Another material may be used as long as the substrate functions as a support in a manufacturing process of the light-emitting elements or the optical elements.

The light-emitting element 150 can be formed using a variety of substrates, for example. The type of the substrate is not limited to a certain type. As the substrate, a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, a base material film, or the like can be used, for example. Examples of the glass substrate include a barium borosilicate glass substrate, an aluminoborosilicate glass substrate, and a soda lime glass substrate. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is a synthetic resin such as acrylic. Other examples are polypropylene, a polyester, polyvinyl fluoride, polyvinyl chloride, and the like. Other examples are a polyamide, a polyimide, aramid, epoxy, an inorganic film formed by evaporation, paper, and the like.

Alternatively, a flexible substrate may be used as the substrate, and the light-emitting element may be provided directly on the flexible substrate. Alternatively, a separation layer may be provided between the substrate and the light-emitting element. The separation layer can be used when part or the whole of the light-emitting element formed over the separation layer is completed, separated from the substrate, and transferred to another substrate. In such a case, the light-emitting element can be transferred to a substrate having low heat resistance or a flexible substrate as well. For the above separation layer, a stack including inorganic films, which are a tungsten film and a silicon oxide film, or an organic resin film of a polyimide or the like formed over a substrate can be used, for example.

In other words, after the light-emitting element is formed using a substrate, the light-emitting element may be transferred to another substrate. Examples of a substrate to which the light-emitting element is transferred include, in addition to the above-described substrates, a paper substrate, a cellophane substrate, an aramid film substrate, a polyimide film substrate, a stone substrate, a wood substrate, a cloth substrate (including a natural fiber (e.g., silk, cotton, or hemp), a synthetic fiber (e.g., a nylon, a polyurethane, or a polyester), a regenerated fiber (e.g., acetate, cupra, rayon, or regenerated polyester, or the like), a leather substrate, and a rubber substrate. By using such a substrate, a light-emitting element with high durability, a light-emitting element with high heat resistance, a lightweight light-emitting element, or a thin light-emitting element can be obtained.

The light-emitting element 150 may be formed over an electrode electrically connected to a field-effect transistor (FET), for example, which is formed over the above-mentioned substrate, so that an active matrix display device in which the FET controls the drive of the light-emitting element 150 can be manufactured.

Note that the structure described in this embodiment can be used in appropriate combination with any of the other embodiments and examples.

### Embodiment 3

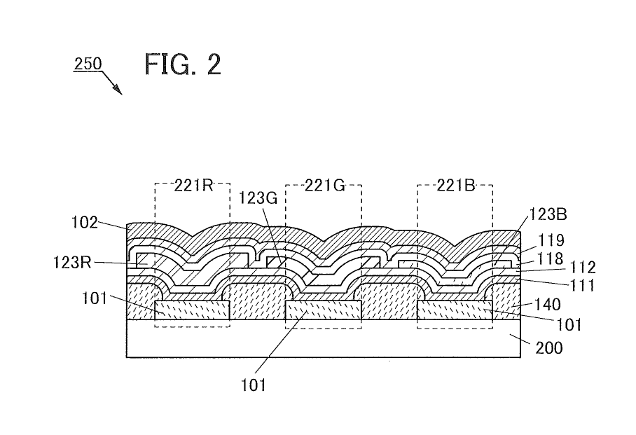

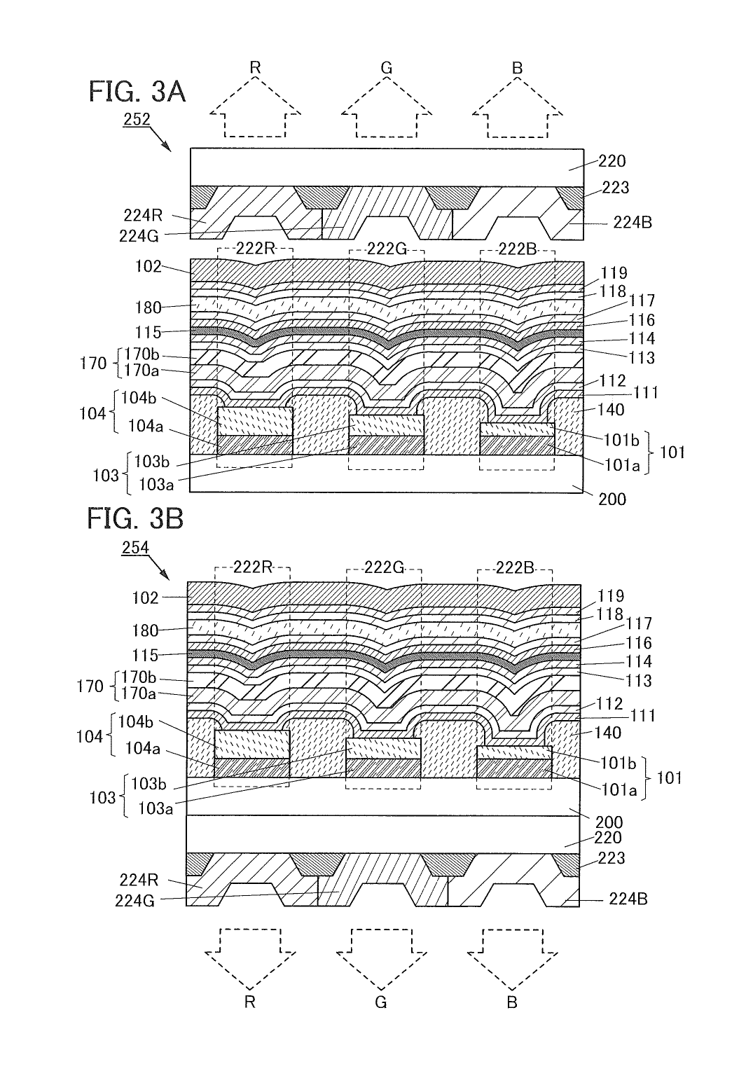

In this embodiment, examples of light-emitting elements having structures different from that described in Embodiment 2 are described below with reference to FIG. 2 and FIGS. 3A and 3B.

### <3-1. Structure 2 of Light-Emitting Element>

FIG. 2 is a cross-sectional view illustrating a light-emitting element of one embodiment of the present invention.

In FIG. 2, a portion having a function similar to that in FIGS. 1A to 1C is represented by the same hatch pattern as in FIGS. 1A to 1C and not especially denoted by a reference numeral in some cases. In addition, common reference numerals are used for portions having similar functions, and a detailed description of the portions is omitted in some cases.

A light-emitting element 250 includes the first electrode 101 and the second electrode 102 over a substrate 200. Between the first and second electrodes 101 and 102, a light-emitting layer 123B, a light-emitting layer 123G, and a light-emitting layer 123R are provided. Furthermore, the light-emitting element 250 includes the hole-injection layer 111, the hole-transport layer 112, the electron-transport layer 118, and the electron-injection layer 119.

The light-emitting element 250 in FIG. 2 has a bottom-emission structure in which light is extracted through the substrate 200. However, one embodiment of the present invention is not limited to this structure and may have a top-emission structure in which light emitted from the light-emitting element is extracted in the direction opposite to the substrate 200 or a dual-emission structure in which light emitted from the light-emitting element is extracted in both top and bottom directions of the substrate 200 over which the light-emitting element is formed.

Since the light-emitting element 250 has a bottom emission structure, the first electrode 101 has a function of transmitting light and the second electrode 102 has a function of reflecting light.

In the light-emitting element 250, a partition 140 is present between a first region 221B and a second region 221G and between the second region 221G and a third region 221R, in each of which the components are interposed between the first and second electrodes 101 and 102. The partition 140 has an insulating property. The partition 140 covers an end portion of the first electrode 101 and has openings overlapping with the electrodes. With the partition 140, the first electrode 101 provided over the substrate 200 in the regions can be divided into island shapes.

The light-emitting layers 123B, 123G, and 123R preferably include light-emitting materials having functions of emitting light of different colors. For example, with the light-emitting layer 123B including a light-emitting material having a function of emitting blue light, the light-emitting layer 123G including a light-emitting material having a function of emitting green light, and the light-emitting layer 123R including a light-emitting material having a function of emitting red light, the light-emitting element 250 can be used in a display device capable of full-color display. The thicknesses of the light-emitting layers may be the same or different.

At least one of the light-emitting layers 123B, 123G, and 123R preferably includes the benzotriphenylene compound described in Embodiment 1. The use of the benzotriphenylene compound described in Embodiment 1 especially in the light-emitting layer 123B enables a light-emitting element with high emission efficiency and an emission spectrum peak in the blue wavelength range.

One or more of the light-emitting layers 123B, 123G, and 123R may include two or more stacked layers.

When at least one light-emitting layer includes the benzotriphenylene compound described in Embodiment 1 as described above and the light-emitting element 250 including the light-emitting layer is used in each sub-pixel of pixels in a display panel, the display panel can have high emission efficiency. The light-emitting device including the light-emitting element 250 can thus have reduced power consumption.

### <3-2. Structure 3 of Light-Emitting Element>

Next, structure examples different from the light-emitting element illustrated in FIG. 2 are described below with reference to FIGS. 3A and 3B.

FIGS. 3A and 3B are cross-sectional views illustrating light-emitting elements of embodiments of the present invention. In FIGS. 3A and 3B, a portion having a function similar to those in FIG. 2 is represented by the same hatch pattern as in FIG. 2 and not especially denoted by a reference numeral in some cases. In addition, common reference numerals are used for portions having similar functions, and a detailed description of the portions is omitted in some cases.

FIGS. 3A and 3B each illustrate a structure example of a tandem light-emitting element in which a plurality of light-emitting layers are stacked between a pair of electrodes with a charge-generation layer 115 provided between the light-emitting layers. A light-emitting element 252 illustrated in FIG. 3A has a top-emission structure in which light is extracted in a direction opposite to the substrate 200, and a light-emitting element 254 illustrated in FIG. 3B has a bottom-emission structure in which light is extracted to the substrate 200 side.

The light-emitting elements 252 and 254 each include the first electrode 101, the second electrode 102, a third electrode 103, and a fourth electrode 104 over the substrate 200. A first light-emitting layer 170, the charge-generation layer 115, and a second light-emitting layer 180 are provided between the first electrode 101 and the second electrode 102, between the second electrode 102 and the third electrode 103, and between the second electrode 102 and the fourth electrode 104. The light-emitting element 252 and the light-emitting element 254 each include the hole-injection layer 111, the hole-transport layer 112, the electron-transport layer 113, the electron-injection layer 114, the hole-injection layer 116, the hole-transport layer 117, the electron-transport layer 118, and the electron-injection layer 119.

The first electrode 101 includes a conductive layer 101a and a conductive layer 101b over the conductive layer 101a . The third electrode 103 includes a conductive layer 103a and a conductive layer 103b over the conductive layer 103a . The fourth electrode 104 includes a conductive layer 104a and a conductive layer 104b over the conductive layer 104a.

In the light-emitting element 252 illustrated in FIG. 3A and the light-emitting element 254 illustrated in FIG. 3B, the partition 140 is present between a first region 222B in which the components are interposed between the first and second electrodes 101 and 102 and a second region 222G in which the components are interposed between the second and third electrodes 102 and 103. The partition 140 is present also between the second region 222G and a third region 222R in which the components are interposed between the second and fourth electrodes 102 and 104. The partition 140 has an insulating property. The partition 140 covers end portions of the first, third, and fourth electrodes 101, 103, and 104 and has openings overlapping with the electrodes. With the partition 140, the electrodes over the substrate 200 in the regions can be divided into island shapes.

The light-emitting elements 252 and 254 each include a substrate 220 provided with a first optical element 224B, a second optical element 224G, and a third optical element 224R in the direction in which light emitted from the first region 222B, light emitted from the second region 222G, and light emitted from the third region 222R are extracted. The light emitted from each region is emitted outside the light-emitting element through each optical element. In other words, the light from the first region 222B, the light from the second region 222G, and the light from the third region 222R are emitted through the first optical element 224B, the second optical element 224G, and the third optical element 224R, respectively.

The first, second, and third optical elements 224B, 224G, and 224R each have a function of selectively transmitting light of a particular color out of incident light. For example, the light emitted from the first region 222B through the first optical element 224B is blue light, the light emitted from the second region 222G through the second optical element 224G is green light, and the light emitted from the third region 222R through the third optical element 224R is red light.

Note that in FIGS. 3A and 3B, blue light (B), green light (G), and red light (R) emitted from the regions through the optical elements are schematically illustrated by the arrows of dashed lines.

A light-blocking layer 223 is provided between the optical elements. The light-blocking layer 223 has a function of blocking light emitted from the adjacent regions. Note that a structure without the light-blocking layer 223 may also be employed.

Furthermore, the light-emitting elements 252 and 254 each have a microcavity structure.

Light emitted from the first and second light-emitting layers 170 and 180 resonates between a pair of electrodes (e.g., the first electrode 101 and the second electrode 102). In each of the light-emitting elements 252 and 254, the thicknesses of the conductive layers (the conductive layer 101b , the conductive layer 103b , and the conductive layer 104b ) in each region are adjusted so that the wavelength of light emitted from the first and second light-emitting layers 170 and 180 can be intensified. Note that the thickness of at least one of the hole-injection layer 111 and the hole-transport layer 112 may differ between the regions so that the wavelength of light emitted from the second and first light-emitting layers 180 and 170 is intensified.

For example, in the case where the refractive index of a substance of the conductive layer 101a having a function of reflecting light between the first and second electrodes 101 and 102 is lower than the refractive index of the first and second light-emitting layers 170 and 180, the thickness of the conductive layer 101b of the first electrode 101 is adjusted so that the optical path length between the first electrode 101 and the second electrode 102 is mλ_{B}/2 (in is a natural number and λ_{B} is a wavelength of light which is intensified in the first region 222B). Similarly, the thickness of the conductive layer 103b of the third electrode 103 is adjusted so that the optical path length between the third electrode 103 and the second electrode 102 is mλ_{G}/2 (m is a natural number and λ_{G} is a wavelength of light which is intensified in the second region 222G). Furthermore, the thickness of the conductive layer 104b of the fourth electrode 104 is adjusted so that the optical path length between the fourth electrode 104 and the second electrode 102 is mλ_{R}/2 (in is a natural number and λ_{R} is a wavelength of light which is intensified in the third region 222R).

In the above manner, with the microcavity structure, in which the optical path length between the pair of electrodes in the respective regions is adjusted, scattering and absorption of light in the vicinity of the electrodes can be suppressed, resulting in high light extraction efficiency. In the above structure, each of the conductive layers 101b, 103b, and 104b preferably has a function of transmitting light. The materials of the conductive layers 101b, 103b, and 104b may be the same or different. The conductive layers 101b, 103b, and 104b may each have two or more stacked layers.

Note that since the light-emitting element 252 illustrated in FIG. 3A has a top-emission structure, it is preferable that the conductive layer 101a of the first electrode 101, the conductive layer 103a of the third electrode 103, and the conductive layer 104a of the fourth electrode 104 have a function of reflecting light. In addition, it is preferable that the second electrode 102 have functions of transmitting light and reflecting light.

Since the light-emitting element 254 illustrated in FIG. 3B has a bottom-emission structure, it is preferable that the conductive layer 101a of the first electrode 101, the conductive layer 103a of the third electrode 103, and the conductive layer 104a of the fourth electrode 104 have functions of transmitting light and reflecting light. In addition, it is preferable that the second electrode 102 have a function of reflecting light.

Materials used for the conductive layers 101a , 103a, and 104a may be the same or different in each of the light-emitting elements 252 and 254. When the conductive layers 101a , 103a, and 104a are formed using the same materials, manufacturing cost of the light-emitting elements 252 and 254 can be reduced. The conductive layers 101a , 103a, and 104a may each have two or more stacked layers.

At least one of the first and second light-emitting layers 170 and 180 preferably includes the benzotriphenylene compound described in Embodiment 1, in which case a light-emitting element in which the delayed fluorescence components account for a high percentage of light emitted from the light-emitting layers can be fabricated. Particularly in the first region 222B, the light-emitting element can have high emission efficiency and an emission spectrum peak in the blue wavelength range.

The first and second light-emitting layers 170 and 180 can each have a stacked-layer structure of two layers, for example, a light-emitting layer 170a and a light-emitting layer 170b . Two kinds of light-emitting materials (a first compound and a second compound) having functions of emitting light of different colors are used in the two light-emitting layers, so that light of a plurality of emission colors can be obtained at the same time. It is particularly preferable to select light-emitting materials so that white light can be obtained by combining light emission from the first and second light-emitting layers 170 and 180.

The first light-emitting layer 170 or the second light-emitting layer 180 may have a structure in which three or more layers are stacked or may include a layer containing no light-emitting material.

When at least one light-emitting layer includes the benzotriphenylene compound described in Embodiment 1 as described above and the light-emitting element 252 or the light-emitting element 254 including the light-emitting layer is used in each sub-pixel of pixels in a display panel, the display panel can have high emission efficiency. The light-emitting device including the light-emitting element 252 or the light-emitting element 254 can thus have reduced power consumption.

Note that the structure described in this embodiment can be combined as appropriate with any of the structures described in the other embodiments.

### Embodiment 4

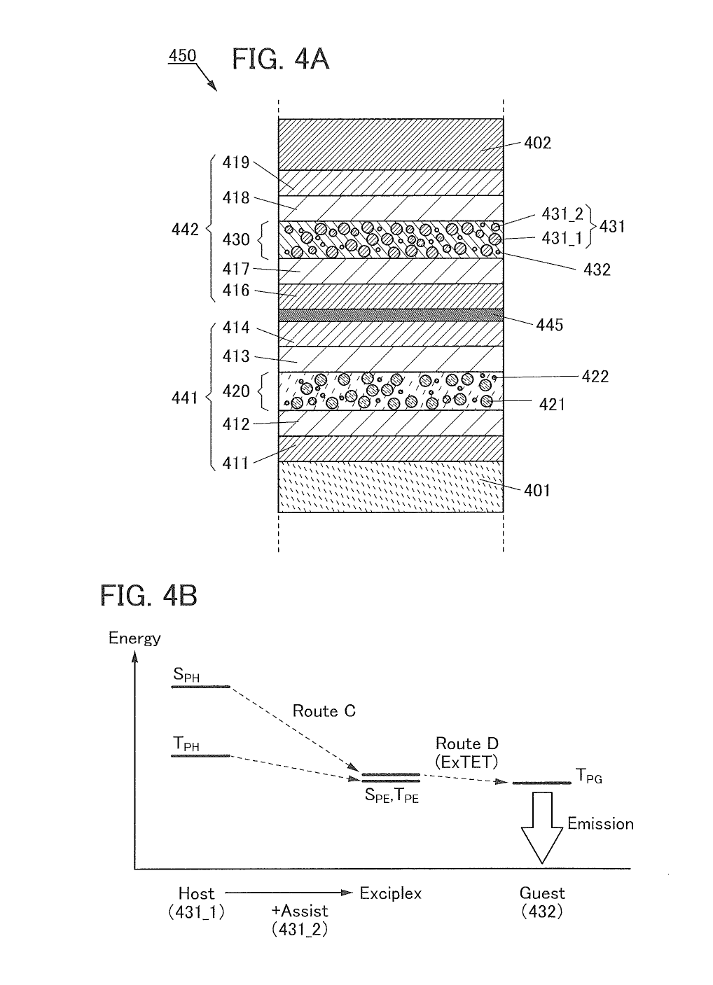

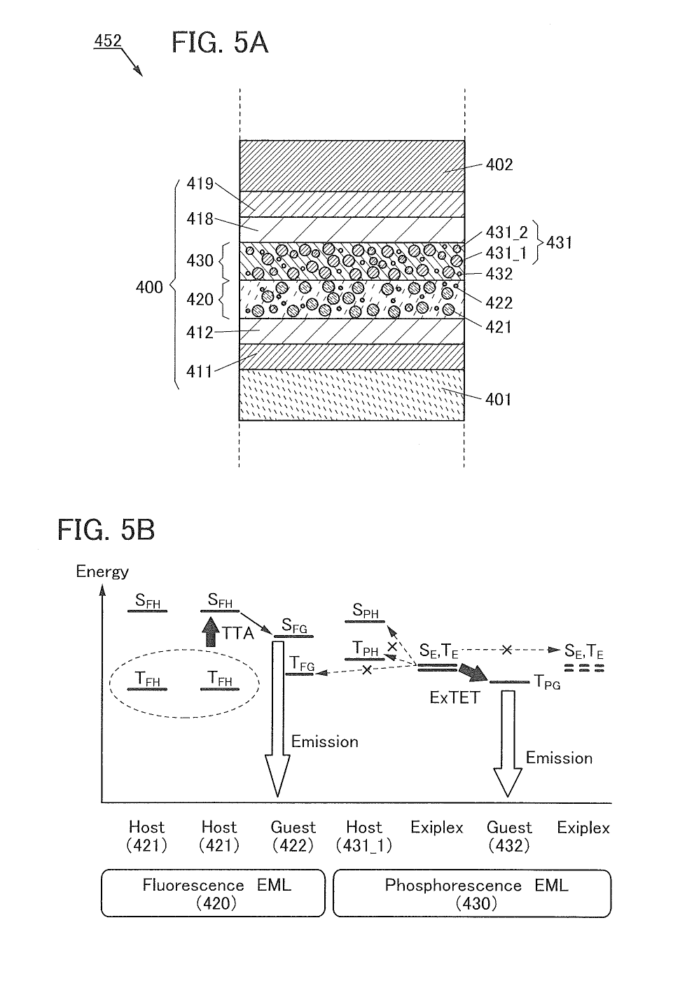

In this embodiment, light-emitting elements having structures different from those described in Embodiments 2 and 3 and emission mechanisms of the light-emitting elements are described below with reference to FIGS. 4A and 4B and FIGS. 5A and 5B.

### <4-1. Structure Example 1 of Light-Emitting Element>

FIG. 4A is a schematic cross-sectional view of a light-emitting element 450.

The light-emitting element 450 illustrated in FIG. 4A includes a plurality of light-emitting units (a first light-emitting unit 441 and a second light-emitting unit 442 in FIG. 4A) between a pair of electrodes (a first electrode 401 and a second electrode 402). One light-emitting unit has the same structure as the EL layer 100 illustrated in FIG. 1A. That is, the light-emitting element 150 in FIG. 1A includes one light-emitting unit, while the light-emitting element 450 includes a plurality of light-emitting units. Note that the first electrode 401 functions as an anode and the second electrode 402 functions as a cathode in the following description of the light-emitting element 450.

In the light-emitting element 450 illustrated in FIG. 4A, the first light-emitting unit 441 and the second light-emitting unit 442 are stacked, and a charge-generation layer 445 is provided between the first light-emitting unit 441 and the second light-emitting unit 442. Note that the first light-emitting unit 441 and the second light-emitting unit 442 may have the same structure or different structures. For example, it is preferable that the EL layer 100 illustrated in FIG. 1A be used in the first light-emitting unit 441 and that a light-emitting layer containing a phosphorescent material as a light-emitting material be used in the second light-emitting unit 442.

That is, the light-emitting element 450 includes a first light-emitting layer 420 and a second light-emitting layer 430. The first light-emitting unit 441 includes a hole-injection layer 411, a hole-transport layer 412, an electron-transport layer 413, and an electron-injection layer 414 in addition to the first light-emitting layer 420. The second light-emitting unit 442 includes a hole-injection layer 416, a hole-transport layer 417, an electron-transport layer 418, and an electron-injection layer 419 in addition to the second light-emitting layer 430.

The charge-generation layer 445 contains a composite material of an organic compound and a metal oxide. For the composite material, the composite material that can be used for the hole-injection layer 111 described above may be used. As the organic compound, a variety of compounds such as an aromatic amine compound, a carbazole compound, an aromatic hydrocarbon, and a high molecular compound (such as an oligomer, a dendrimer, or a polymer) can be used. An organic compound having a hole mobility of 1×10⁶ cm²/Vs or higher is preferably used. Note that any other material may be used as long as it has a property of transporting more holes than electrons. Since the composite material of an organic compound and a metal oxide has excellent carrier-injection and carrier-transport properties, low-voltage driving or low-current driving can be realized. Note that when a surface of a light-emitting unit on the anode side is in contact with the charge-generation layer 445, the charge-generation layer 445 can also serve as a hole-transport layer of the light-emitting unit; thus, a hole-transport layer does not need to be included in the light-emitting unit.

The charge-generation layer 445 may have a stacked-layer structure of a layer containing the composite material of an organic compound and a metal oxide and a layer containing another material. For example, the charge-generation layer 445 may be formed using a combination of a layer containing the composite material of an organic compound and a metal oxide with a layer containing one compound selected from among materials having an electron donating property and a compound having a high electron-transport property. Furthermore, the charge-generation layer 445 may be formed using a combination of a layer containing the composite material of an organic compound and a metal oxide with a transparent conductive film.

The charge-generation layer 445 provided between the first light-emitting unit 441 and the second light-emitting unit 442 may have any structure as long as electrons can be injected to the light-emitting unit on one side and holes can be injected into the light-emitting unit on the other side when a voltage is applied between the first electrode 401 and the second electrode 402. For example, in FIG. 4A, the charge-generation layer 445 injects electrons into the first light-emitting unit 441 and holes into the second light-emitting unit 442 when a voltage is applied such that the potential of the first electrode 401 is higher than that of the second electrode 402.

The light-emitting element having two light-emitting units is described with reference to FIG. 4A; however, a similar structure can be applied to a light-emitting element in which three or more light-emitting units are stacked. With a plurality of light-emitting units partitioned by the charge-generation layer between a pair of electrodes as in the light-emitting element 450, it is possible to provide a light-emitting element which can emit light with high luminance with the current density kept low and has a long lifetime. A light-emitting element that can be driven at a low voltage and has low power consumption can be manufactured.

When the structure of the EL layer 100 is applied to at least one of the plurality of light-emitting units, a light-emitting element with high emission efficiency can be provided. In particular, use of the benzotriphenylene compound of one embodiment of the present invention in the light-emitting layer of at least one light-emitting unit can provide a light-emitting element with high emission efficiency.

The first light-emitting layer 420 includes a host material 421 and a guest material 422. The second light-emitting layer 430 includes a host material 431 and a guest material 432. The host material 431 includes a first organic compound 431_1 and a second organic compound 431_2.

In this embodiment, the first light-emitting layer 420 has a structure similar to that of the light-emitting layer 130 in FIGS. 1A to 1C. That is, in the first light-emitting layer 420, the host material 421 and the guest material 422 correspond to the host material 131 and the guest material 132, respectively, in the light-emitting layer 130. In the following description, the guest material 432 included in the second light-emitting layer 430 is a phosphorescent material. Note that the first electrode 401, the second electrode 402, the hole-injection layers 411 and 416, the hole-transport layers 412 and 417, the electron-transport layers 413 and 418, and the electron-injection layers 414 and 419 correspond to the first electrode 101, the second electrode 102, the hole-injection layer 111, the hole-transport layer 112, the electron-transport layer 118, and the electron-injection layer 119 in Embodiment 1, respectively. Therefore, detailed description thereof is omitted in this embodiment.

### <4-2. Emission Mechanism of First Light-Emitting Layer>

An emission mechanism of the first light-emitting layer 420 is similar to that of the light-emitting layer 130 in FIGS. 1A to 1C.

### <4-3. Emission Mechanism of Second Light-Emitting Layer>

Next, an emission mechanism of the second light-emitting layer 430 is described.

The first organic compound 431_1 and the second organic compound 431_2 which are included in the second light-emitting layer 430 form an exciplex (an excited complex). The first organic compound 431_1 serves as a host material and the second organic compound 431_2 serves as an assist material in the description here.

Although it is acceptable as long as the combination of the first organic compound 431_1 and the second organic compound 431_2 can form an exciplex in the second light-emitting layer 430, it is preferred that one organic compound be a material having a hole-transport property and the other organic compound be a material having an electron-transport property.

FIG. 4B illustrates the correlation of energy levels of the first organic compound 431_1, the second organic compound 431_2, and the guest material 432 in the second light-emitting layer 430. The following explains what terms and signs in FIG. 4B represent.

Host: the host material (the first organic compound 431_1)

Assist: the assist material (the second organic compound 431_2)

Guest: the guest material 432 (phosphorescent material)

S_{PH}: the level of the lowest singlet excited state of the host material (the first organic compound 431_1)

T_{PH}: the level of the lowest triplet excited state of the host material (the first organic compound 431_1)

T_{PG}: the level of the lowest triplet excited state of the guest material 432 (phosphorescent material)

S_{PE}: the level of the lowest singlet excited state of the exciplex

T_{PE}: the level of the lowest triplet excited state of the exciplex

The level (S_{PE}) of the lowest singlet excited state of the exciplex formed by the first and second organic compounds 431_1 and 431_2 and the level (T_{PE}) of the lowest triplet excited state of the exciplex are close to each other (see Route C in FIG. 4B).

Both energies of S_{PE} and T_{PE} of the exciplex are then transferred to the level of the lowest triplet excited state of the guest material 432 (phosphorescent material); thus, light emission is obtained (see Route D in FIG. 4B).

The above-described processes through Route C and Route D may be referred to as exciplex-triplet energy transfer (ExTET) in this specification and the like.

When one of the first and second organic compounds 431_1 and 431_2 receiving holes and the other receiving electrons come close to each other, an exciplex is formed at once. Alternatively, when one compound is brought into an excited state, the one immediately interacts with the other compound to form an exciplex. Therefore, most excitons in the second light-emitting layer 430 exist as exciplexes. The band gap of the exciplex is narrower than that of each of the first and second organic compounds 431_1 and 431_2; therefore, the drive voltage can be lowered when the exciplex is formed by recombination of a hole and an electron.

When the second light-emitting layer 430 has the above structure, light emission from the guest material 432 (phosphorescent material) of the second light-emitting layer 430 can be efficiently obtained.

Note that light emitted from the first light-emitting layer 420 preferably has a peak on the shorter wavelength side than light emitted from the second light-emitting layer 430. Since the luminance of a light-emitting element using a phosphorescent material emitting light with a short wavelength tends to be degraded quickly, fluorescence with a short wavelength is employed so that a light-emitting element with less degradation of luminance can be provided.

Furthermore, the first light-emitting layer 420 and the second light-emitting layer 430 may be made to emit light with different emission wavelengths, so that the light-emitting element can be a multicolor light-emitting element. In that case, the emission spectrum of the light-emitting element is formed by combining light having different emission peaks, and thus has at least two peaks.

The above structure is also suitable for obtaining white light emission. When the first light-emitting layer 420 and the second light-emitting layer 430 emit light of complementary colors, white light emission can be obtained.

In addition, white light emission with a high color rendering property that is formed of three primary colors or four or more colors can be obtained by using a plurality of light-emitting substances emitting light with different wavelengths for one of the first and second light-emitting layers 420 and 430 or both. In that case, one of the first and second light-emitting layers 420 and 430 or both may be divided into layers and each of the divided layers may contain a light-emitting material different from the others.

Next, materials that can be used in the first and second light-emitting layers 420 and 430 are described.

[Material that can be used in First Light-Emitting Layer]

As a material that can be used in the first light-emitting layer 420, a material that can be used in the light-emitting layer 130 in Embodiment 1 may be used.

[Material that can be used in Second Light-Emitting Layer]

In the second light-emitting layer 430, the first organic compound 431_1 (host material) exists in the highest proportion in weight ratio, and the guest material 432 (phosphorescent material) is dispersed in the first organic compound 431_1 (host material).

Examples of the first organic compound 431_1 (host material) include a zinc- or aluminum-based metal complex, an oxadiazole derivative, a triazole derivative, a benzimidazole derivative, a quinoxaline derivative, a dibenzoquinoxaline derivative, a dibenzothiophene derivative, a dibenzofuran derivative, a pyrimidine derivative, a triazine derivative, a pyridine derivative, a bipyridine derivative, a phenanthroline derivative, and the like. Other examples are an aromatic amine, a carbazole derivative, and the like.

As the guest material 432 (phosphorescent material), an iridium-, rhodium-, or platinum-based organometallic complex or metal complex can be used; in particular, an organoiridium complex such as an iridium-based ortho-metalated complex is preferable. As an ortho-metalated ligand, a 4H-triazole ligand, a 1H-triazole ligand, an imidazole ligand, a pyridine ligand, a pyrimidine ligand, a pyrazine ligand, an isoquinoline ligand, and the like can be given. As the metal complex, a platinum complex having a porphyrin ligand and the like can be given.

As the second organic compound 431_2 (assist material), a substance which can form an exciplex together with the first organic compound 431_1 is used. In that case, it is preferable that the first organic compound 431_1, the second organic compound 431_2, and the guest material 432 (phosphorescent material) be selected such that the emission peak of the exciplex overlaps with an absorption band, specifically an absorption band on the longest wavelength side, of a triplet metal to ligand charge transfer (MLCT) transition of the guest material 432 (phosphorescent material). This makes it possible to provide a light-emitting element with drastically improved emission efficiency. Note that in the case where a thermally activated delayed fluorescent material is used instead of the phosphorescent material, it is preferable that the absorption band on the longest wavelength side be a singlet absorption band.

As the light-emitting material contained in the second light-emitting layer 430, any material can be used as long as the material can convert triplet excited energy into light emission. As an example of the material that can convert triplet excited energy into light emission, a thermally activated delayed fluorescent (TADF) material can be given in addition to the phosphorescent material. Therefore, the term “phosphorescent material” in the description can be replaced with the term “thermally activated delayed fluorescent material”. Note that the thermally activated delayed fluorescent material is a material that can up-convert a triplet excited state into a singlet excited state (i.e., reverse intersystem crossing) using a little thermal energy and efficiently exhibits light emission (fluorescence) from the singlet excited state. Thermally activated delayed fluorescence is efficiently obtained under the condition where the difference between the triplet excited energy level and the singlet excited energy level is larger than 0 eV and smaller than or equal to 0.2 eV, further preferably larger than 0 eV and smaller than or equal to 0.1 eV.

The material that emits thermally activated delayed fluorescence may be a material that can form a singlet excited state by itself from a triplet excited state by reverse intersystem crossing or may be a combination of two kinds of materials which form an exciplex.

There is no limitation on the emission colors of the light-emitting material included in the first light-emitting layer 420 and the light-emitting material included in the second light-emitting layer 430, and they may be the same or different. Light emitted from the light-emitting materials is mixed and extracted out of the element; therefore, for example, in the case where their emission colors are complementary colors, the light-emitting element can emit white light. In consideration of the reliability of the light-emitting element, the emission peak wavelength of the light-emitting material included in the first light-emitting layer 420 is preferably shorter than that of the light-emitting material included in the second light-emitting layer 430.

### <4-4. Structure Example 2 of Light-Emitting Element>

Next, a structure example different from the light-emitting element illustrated in FIGS. 4A and 4B is described below with reference to FIGS. 5A and 5B.

FIG. 5A is a schematic cross-sectional view of a light-emitting element 452.

In the light-emitting element 452, an EL layer 400 is provided between a pair of electrodes (the first electrode 401 and the second electrode 402). Note that in the light-emitting element 452, the first electrode 401 functions as an anode, and the second electrode 402 functions as a cathode.

The EL layer 400 includes the first and second light-emitting layers 420 and 430. As the EL layer 400 in the light-emitting element 450, the first and second light-emitting layers 420 and 430, the hole-injection layer 411, the hole-transport layer 412, the electron-transport layer 418, and the electron-injection layer 419 are illustrated. However, this stacked-layer structure is an example, and the structure of the EL layer 400 in the light-emitting element 450 is not limited thereto. For example, the stacking order of the above layers of the EL layer 400 may be changed. Alternatively, in the EL layer 400, another functional layer other than the above layers may be provided. The functional layer may have a function of injecting a carrier (an electron or a hole), a function of transporting a carrier, a function of inhibiting a carrier, or a function of generating a carrier, for example.

The first light-emitting layer 420 includes the host material 421 and the guest material 422. The second light-emitting layer 430 includes the host material 431 and the guest material 432. The host material 431 includes the first organic compound 431_1 and the second organic compound 431_2. In the following description, the guest material 422 is a fluorescent material and the guest material 432 is a phosphorescent material.

### <4-5. Emission Mechanism of First Light-Emitting Layer>

The emission mechanism of the first light-emitting layer 420 is similar to that of the light-emitting layer 130 in FIGS. 1A to 1C.

### <4-6. Emission Mechanism of Second Light-Emitting Layer>

The emission mechanism of the second light-emitting layer 430 is similar to that of the light-emitting layer 430 in FIGS. 4A and 4B.

### <4-7. Emission Mechanism of First and Second Light-Emitting Layers>

Each emission mechanism of the first and second light-emitting layers 420 and 430 is described above. As in the light-emitting element 452, in the case where the first and second light-emitting layers 420 and 430 are in contact with each other, even when energy is transferred from the exciplex to the host material 421 of the first light-emitting layer 420 (in particular, when energy of the triplet excited level is transferred) at an interface between the first light-emitting layer 420 and the second light-emitting layer 430, triplet excited energy can be converted into light emission in the first light-emitting layer 420.

The T₁ level of the host material 421 of the first light-emitting layer 420 is preferably lower than T₁ levels of the first and second organic compounds 431_1 and 431_2 in the second light-emitting layer 430. In the first light-emitting layer 420, the S₁ level of the host material 421 is preferably higher than the S₁ level of the guest material 422 (fluorescent material) while the T₁ level of the host material 421 is preferably lower than the T₁ level of the guest material 422 (fluorescent material).

FIG. 5B shows the correlation of energy levels in the case where TTA is utilized in the first light-emitting layer 420 and ExTET is utilized in the second light-emitting layer 430. The following explains what terms and signs in FIG. 5B represent.

Fluorescence EML: the fluorescent light-emitting layer (first light-emitting layer 420)

Phosphorescence EMI,: the phosphorescent light-emitting layer (second light-emitting layer 430)

S_{FH}: the level of the lowest singlet excited state of the host material 421

T_{FH}: the level of the lowest triplet excited state of the host material 421

S_{FG}: the level of the lowest singlet excited state of the guest material 422 (fluorescent material)

T_{FG}: the level of the lowest triplet excited state of the guest material 422 (fluorescent material)

S_{PH}: the level of the lowest singlet excited state of the host material (first organic compound 431_1)

T_{PH}: the level of the lowest triplet excited state of the host material (first organic compound 431_1)

T_{PG}: the level of the lowest triplet excited state of the guest material 432 (phosphorescent material)

S_{E}: the level of the lowest singlet excited state of the exciplex

T_{B}: the level of the lowest triplet excited state of the exciplex

As shown in FIG. 5B, the exciplex exists only in an excited state; thus, exciton diffusion between the exciplexes is less likely to occur. In addition, because the excited levels (S_{E} and T_{E}) of the exciplex are lower than the excited levels (S_{PH} and T_{PN}) of the first organic compound 431_1 (the host material for the phosphorescent material) of the second light-emitting layer 430, energy diffusion from the exciplex to the first organic compound 431_1 does not occur. That is, efficiency of the phosphorescent light-emitting layer (second light-emitting layer 430) can be maintained because an exciton diffusion distance of the exciplex is short in the phosphorescent light-emitting layer (second light-emitting layer 430). In addition, even when part of the triplet excited energy of the exciplex of the phosphorescent light-emitting layer (second light-emitting layer 430) diffuses into the fluorescent light-emitting layer (first light-emitting layer 420) through the interface between the fluorescent light-emitting layer (first light-emitting layer 420) and the phosphorescent light-emitting layer (second light-emitting layer 430), energy loss can be reduced because the triplet excited energy in the fluorescent light-emitting layer (first light-emitting layer 420) caused by the diffusion is used for light emission through TTA.

The light-emitting element 452 can have high emission efficiency because ExTET is utilized in the second light-emitting layer 430 and TTA is utilized in the first light-emitting layer 420 as described above so that energy loss is reduced. As in the light-emitting element 452, in the case where the first light-emitting layer 420 and the second light-emitting layer 430 are in contact with each other, the number of EL layers 400 as well as the energy loss can be reduced. Therefore, a light-emitting element with low manufacturing cost can be obtained.

Note that the first light-emitting layer 420 and the second light-emitting layer 430 need not be in contact with each other. In that case, it is possible to prevent energy transfer by the Dexter mechanism (particularly triplet energy transfer) from the first organic compound 431_1 in an excited state or the guest material 432 (phosphorescent material) in an excited state which is generated in the second light-emitting layer 430 to the host material 421 or the guest material 422 (fluorescent material) in the first light-emitting layer 420. Therefore, the thickness of a layer provided between the first light-emitting layer 420 and the second light-emitting layer 430 may be several nanometers.

The layer provided between the first light-emitting layer 420 and the second light-emitting layer 430 may contain a single material or both a hole-transport material and an electron-transport material. In the case of a single material, a bipolar material may be used. The bipolar material here refers to a material in which the ratio between the electron mobility and the hole mobility is 100 or less. Alternatively, the hole-transport material, the electron-transport material, or the like may be used. At least one of materials included in the layer may be the same as the host material (first organic compound 431_1) of the second light-emitting layer 430. This facilitates the manufacture of the light-emitting element and reduces the drive voltage. Furthermore, the hole-transport material and the electron-transport material may form an exciplex, which effectively prevents exciton diffusion. Specifically, it is possible to prevent energy transfer from the host material (first organic compound 431_1) in an excited state or the guest material 432 (phosphorescent material) in an excited state of the second light-emitting layer 430 to the host material 421 or the guest material 422 (fluorescent material) in the first light-emitting layer 420.

Note that in the light-emitting element 452, a carrier recombination region is preferably distributed to some extent. Therefore, it is preferable that the first light-emitting layer 420 or the second light-emitting layer 430 have an appropriate degree of carrier-trapping property. It is particularly preferable that the guest material 432 (phosphorescent material) in the second light-emitting layer 430 have an electron-trapping property.

Note that light emitted from the first light-emitting layer 420 preferably has a peak on the shorter wavelength side than light emitted from the second light-emitting layer 430. Since the luminance of a light-emitting element using a phosphorescent material emitting light with a short wavelength tends to be degraded quickly, fluorescence with a short wavelength is employed so that a light-emitting element with less degradation of luminance can be provided.

Furthermore, the first light-emitting layer 420 and the second light-emitting layer 430 may be made to emit light with different emission wavelengths, so that the light-emitting element can be a multicolor light-emitting element. In that case, the emission spectrum of the light-emitting element is formed by combining light having different emission peaks, and thus has at least two peaks.

The above structure is also suitable for obtaining white light emission. When the first light-emitting layer 420 and the second light-emitting layer 430 emit light of complementary colors, white light emission can be obtained.

In addition, white light emission with a high color rendering property that is formed of three primary colors or four or more colors can be obtained by using a plurality of light-emitting substances emitting light with different wavelengths for the first light-emitting layer 420. In that case, the first light-emitting layer 420 may be divided into layers and each of the divided layers may contain a light-emitting material different from the others.

Next, materials that can be used in the first and second light-emitting layers 420 and 430 are described.

[Material that can be used in First Light-Emitting Layer]

In the first light-emitting layer 420, the host material 421 is present in the highest proportion by weight, and the guest material 422 (fluorescent material) is dispersed in the host material 421. The Sᵣ level of the host material 421 is preferably higher than the S₁ level of the guest material 422 (fluorescent material) while the T₁ level of the host material 421 is preferably lower than the T₁ level of the guest material 422 (fluorescent material).

As the host material 421, the benzotriphenylene compound described in Embodiment 1 is preferably used to fabricate a light-emitting element with high emission efficiency in which delayed fluorescence accounts for a large proportion of emitted light.

[Materials that can be used in Second Light-Emitting Layer]

In the second light-emitting layer 430, the host material (first organic compound 431_1) is present in the highest proportion by weight, and the guest material 432 (phosphorescent material) is dispersed in the host material (first organic compound 431_1). The T₁ level of the host material (first organic compound 431_1) of the second light-emitting layer 430 is preferably higher than the T₁ level of the guest material 422 (fluorescent material) of the first light-emitting layer 420.

As the host materials (first and second organic compounds 431_1 and 431_2) and the guest material 432 (phosphorescent material), those in the light-emitting element 450 described with reference to FIGS. 4A and 4B can be used.

Note that the structure described in this embodiment can be combined as appropriate with any of the structures described in the other embodiments.

### Embodiment 5

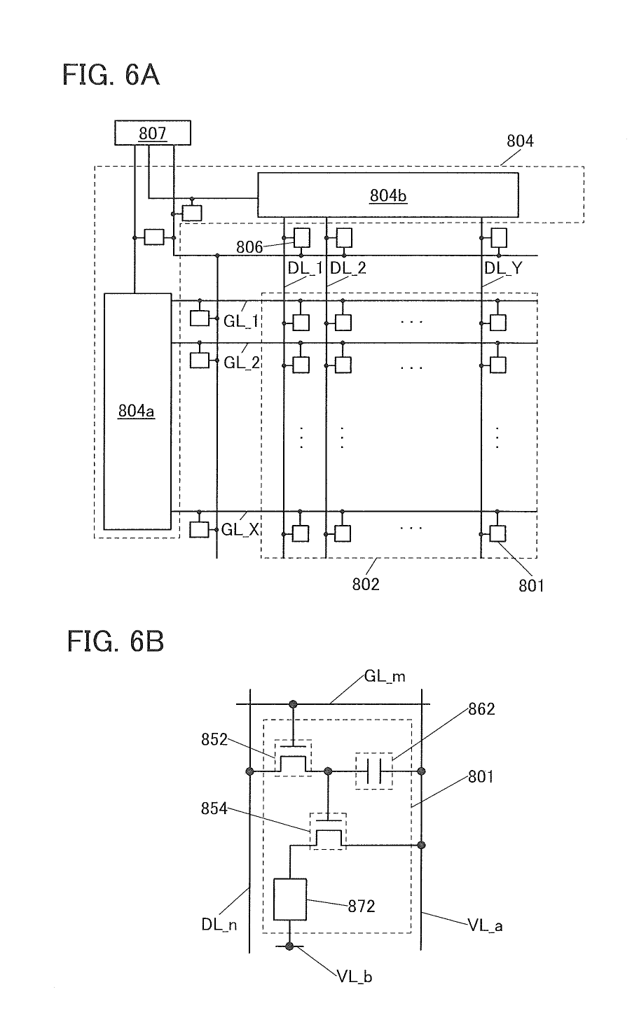

In this embodiment, a display device including a light-emitting element of one embodiment of the present invention is described with reference to FIGS. 6A and 6B, FIGS. 7A and 7B, and FIGS. 8A and 8B.

### <5-1. Structure of Display Device>

FIG. 6A is a block diagram illustrating the configuration of a display device of one embodiment of the present invention.

The display device illustrated in FIG. 6A includes a region including pixels of display elements (the region is hereinafter referred to as a pixel portion 802), a circuit portion provided outside the pixel portion 802 and including circuits for driving the pixels (the portion is hereinafter referred to as a driver circuit portion 804), circuits having a function of protecting elements (the circuits are hereinafter referred to as protection circuits 806), and a terminal portion 807. Note that the protection circuits 806 are not necessarily provided.

Part or the whole of the driver circuit portion 804 is preferably formed over a substrate over which the pixel portion 802 is foamed, in which case the number of components and the number of terminals can be reduced. When part or the whole of the driver circuit portion 804 is not formed over the substrate over which the pixel portion 802 is formed, the part or the whole of the driver circuit portion 804 can be mounted by COG or tape automated bonding (TAB).

The pixel portion 802 includes a plurality of circuits for driving display elements arranged in X rows (Xis a natural number of 2 or more) and Y columns (Y is a natural number of 2 or more) (such circuits are hereinafter referred to as pixel circuits 801). The driver circuit portion 804 includes driver circuits such as a circuit for supplying a signal (scan signal) to select a pixel (the circuit is hereinafter referred to as a gate driver 804a ) and a circuit for supplying a signal (data signal) to drive a display element in a pixel (the circuit is hereinafter referred to as a source driver 804b ).

The gate driver 804a includes a shift register or the like. Through the terminal portion 807, the gate driver 804a receives a signal for driving the shift register and outputs a signal. For example, the gate driver 804a receives a start pulse signal, a clock signal, or the like and outputs a pulse signal. The gate driver 804a has a function of controlling the potentials of wirings supplied with scan signals (such wirings are hereinafter referred to as scan lines GL_1 to GL_X). Note that a plurality of gate drivers 804a may be provided to control the scan lines GL_1 to GL_X separately. Alternatively, the gate driver 804a has a function of supplying an initialization signal. Without being limited thereto, the gate driver 804a can supply another signal.

The source driver 804b includes a shift register or the like. The source driver 804b receives a signal (image signal) from which a data signal is derived, as well as a signal for driving the shift register, through the terminal portion 807. The source driver 804b has a function of generating a data signal to be written to the pixel circuit 801 which is based on the image signal. In addition, the source driver 804b has a function of controlling output of a data signal in response to a pulse signal produced by input of a start pulse signal, a clock signal, or the like. Furthermore, the source driver 804b has a function of controlling the potentials of wirings supplied with data signals (such wirings are hereinafter referred to as data lines DL_1 to DL_Y). Alternatively, the source driver 804b has a function of supplying an initialization signal. Without being limited thereto, the source driver 804b can supply another signal.

The source driver 804b includes a plurality of analog switches or the like, for example. The source driver 804b can output, as the data signals, signals obtained by time-dividing the image signal by sequentially turning on the plurality of analog switches.

A pulse signal and a data signal are input to each of the plurality of pixel circuits 801 through one of the plurality of scan lines GL supplied with scan signals and one of the plurality of data lines DL supplied with data signals, respectively. Writing and holding of the data signal to and in each of the plurality of pixel circuits 801 are controlled by the gate driver 804a . For example, to the pixel circuit 801 in the m-th row and the n-th column (in is a natural number of less than or equal to X, and n is a natural number of less than or equal to Y), a pulse signal is input from the gate driver 804a through the scan line GL_m, and a data signal is input from the source driver 804b through the data line DL_n in accordance with the potential of the scan line GL_m.

The protection circuit 806 illustrated in FIG. 6A is connected to, for example, the scan line GL between the gate driver 804a and the pixel circuit 801. Alternatively, the protection circuit 806 is connected to the data line DL between the source driver 804b and the pixel circuit 801. Alternatively, the protection circuit 806 can be connected to a wiring between the gate driver 804a and the terminal portion 807. Alternatively, the protection circuit 806 can be connected to a wiring between the source driver 804b and the tenninal portion 807. Note that the terminal portion 807 means a portion having terminals for inputting power, control signals, and image signals to the display device from external circuits.

The protection circuit 806 is a circuit that electrically connects a wiring connected to the protection circuit to another wiring when a potential out of a certain range is applied to the wiring connected to the protection circuit.

As illustrated in FIG. 6A, the protection circuits 806 are provided for the pixel portion 802 and the driver circuit portion 804, so that the resistance of the display device to overcurrent generated by electrostatic discharge (ESD) or the like can be improved. Note that the configuration of the protection circuits 806 is not limited to that, and for example, a configuration in which the protection circuits 806 are connected to the gate driver 804a or a configuration in which the protection circuits 806 are connected to the source driver 804b may be employed. Alternatively, the protection circuits 806 may be configured to be connected to the terminal portion 807.

### <5-2. Configuration 1 of Pixel Circuit>

Each of the plurality of pixel circuits 801 in FIG. 6A can have a structure illustrated in FIG. 6B, for example.

The pixel circuit 801 illustrated in FIG. 6B includes transistors 852 and 854, a capacitor 862, and a light-emitting element 872.

One of a source electrode and a drain electrode of the transistor 852 is electrically connected to a wiring to which a data signal is supplied (hereinafter referred to as a signal line DL_n). A gate electrode of the transistor 852 is electrically connected to a wiring to which a gate signal is supplied (hereinafter referred to as a scan line GL_m).

The transistor 852 has a function of controlling whether to write a data signal by being turned on or off.

One of a pair of electrodes of the capacitor 862 is electrically connected to a wiring to which a potential is supplied (hereinafter referred to as a potential supply line VL_a), and the other is electrically connected to the other of a source electrode and a drain electrode of the transistor 852.

The capacitor 862 functions as a storage capacitor for storing written data.

One of a source electrode and a drain electrode of the transistor 854 is electrically connected to the potential supply line VL_a. Furthermore, a gate electrode of the transistor 854 is electrically connected to the other of the source electrode and the drain electrode of the transistor 852.

One of an anode and a cathode of the light-emitting element 872 is electrically connected to a potential supply line VL_b, and the other is electrically connected to the other of the source electrode and the drain electrode of the transistor 854.

As the light-emitting element 872, the light-emitting element described in the above embodiment can be used.

Note that a high power supply potential VDD is supplied to one of the potential supply line VL_a and the potential supply line VL_b, and a low power supply potential VSS is supplied to the other.

In the display device including the pixel circuits 801 in FIG. 6B, the pixel circuits 801 are sequentially selected row by row by the gate driver 804a in FIG. 6A, for example, whereby the transistors 852 are turned on and a data signal is written.

When the transistors 852 are turned off, the pixel circuits 801 in which the data has been written are brought into a holding state. Furthermore, the amount of current flowing between the source electrode and the drain electrode of the transistor 854 is controlled in accordance with the potential of the written data signal. The light-emitting element 872 emits light with a luminance corresponding to the amount of flowing current. This operation is sequentially perfonned row by row; thus, an image is displayed.

### <5-3. Configuration 2 of Pixel Circuit>

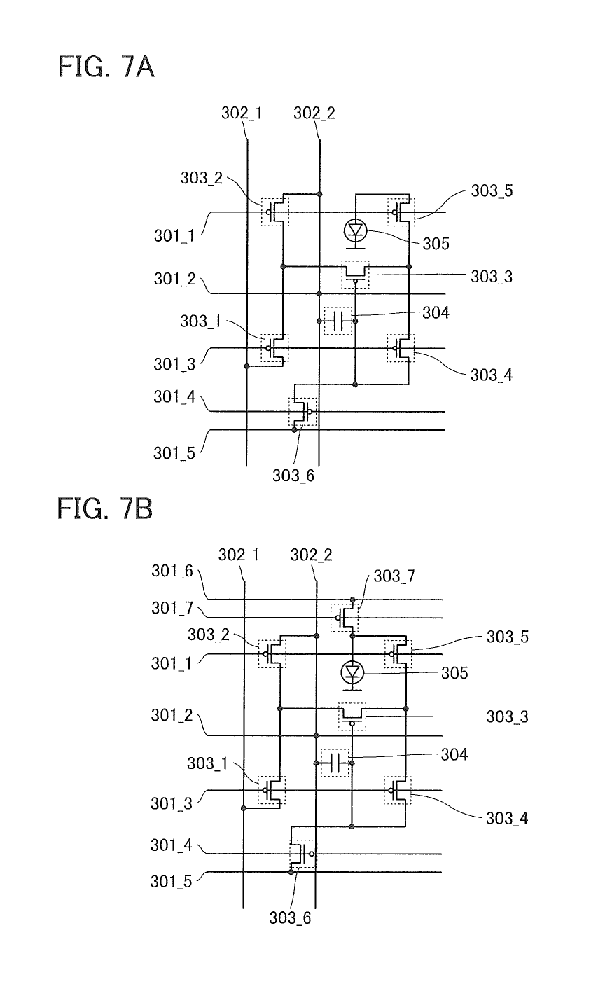

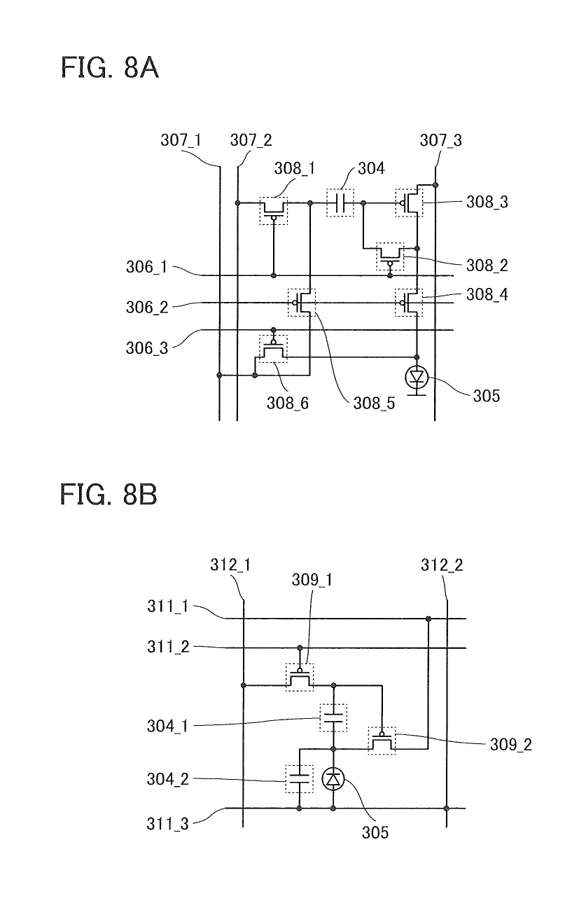

Alternatively, the pixel circuit 801 can have a function of compensating variation in threshold voltages or the like of a transistor. FIGS. 7A and 7B and FIGS. 8A and 8B illustrate configuration examples of the pixel circuit.

The pixel circuit illustrated in FIG. 7A includes six transistors (transistors 303_1 to 303_6), a capacitor 304, and a light-emitting element 305. The pixel circuit illustrated in FIG. 7A is electrically connected to wirings 301_1 to 301_5 and wirings 302_1 and 302_2. Note that as the transistors 303_1 to 303_6, for example, p-channel transistors can be used.

The pixel circuit shown in FIG. 7B has a configuration in which a transistor 303_7 is added to the pixel circuit shown in FIG. 7A. The pixel circuit illustrated in FIG. 7B is electrically connected to wirings 301_6 and 301_7. The wirings 301_5 and 301_6 may be electrically connected to each other. Note that as the transistor 3037, for example, a p-channel transistor can be used.

The pixel circuit illustrated in FIG. 8A includes six transistors (transistors 308_1 to 308_6), the capacitor 304, and the light-emitting element 305. The pixel circuit illustrated in FIG. 8A is electrically connected to wirings 306_1 to 306_3 and wirings 307_1 to 307_3. The wirings 306_1 and 306_3 may be electrically connected to each other. Note that as the transistors 308_1 to 308_6, for example, p-channel transistors can be used.

The pixel circuit illustrated in FIG. 8B includes two transistors (transistors 309_1 and 309_2), two capacitors (capacitors 304_1 and 304_2), and the light-emitting element 305. The pixel circuit illustrated in FIG. 8B is electrically connected to wirings 311_1 to 311_3 and wirings 312_1 and 312_2. With the configuration of the pixel circuit illustrated in FIG. 8B, for example, the light-emitting element 305 can be driven by a voltage inputting current driving method (also referred to as CVCC). Note that as the transistors 309_1 and 309_2, for example, p-channel transistors can be used.

A light-emitting element of one embodiment of the present invention can be used for an active matrix method in which an active element is included in a pixel of a display device or a passive matrix method in which an active element is not included in a pixel of a display device.

In the active matrix method, as an active element (a non-linear element), not only a transistor but also a variety of active elements (non-linear elements) can be used. For example, a metal insulator metal (MIM), a thin film diode (TFD), or the like can also be used. Since these elements can be formed with a smaller number of manufacturing steps, manufacturing cost can be reduced or yield can be improved. Alternatively, since the size of these elements is small, the aperture ratio can be improved, so that power consumption can be reduced or higher luminance can be achieved.

As a method other than the active matrix method, the passive matrix method in which an active element (a non-linear element) is not used can also be used. Since an active element (a non-linear element) is not used, the number of manufacturing steps is small, so that manufacturing cost can be reduced or yield can be improved. Alternatively, since an active element (a non-linear element) is not used, the aperture ratio can be improved, so that power consumption can be reduced or higher luminance can be achieved, for example.

The structure described in this embodiment can be used in appropriate combination with any of the structures described in the other embodiments.

### Embodiment 6

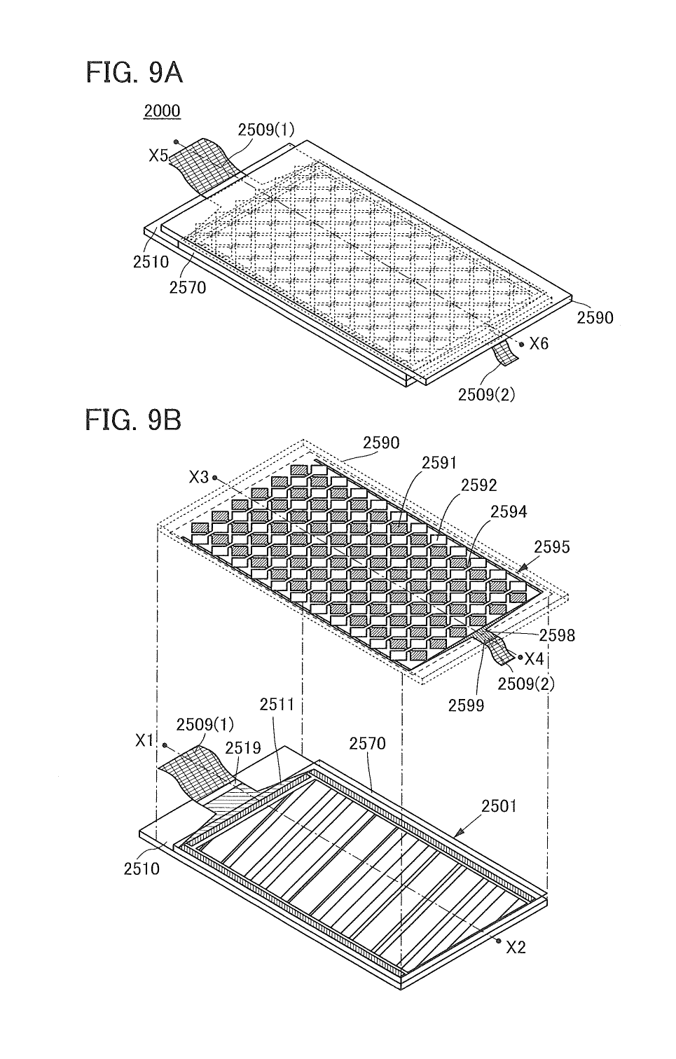

In this embodiment, a display panel including a light-emitting device of one embodiment of the present invention and an electronic device in which the display panel is provided with an input device will be described with reference to FIGS. 9A and 9B, FIGS. 10A to 10C, FIGS. 11A and 11B, FIGS. 12A and 12B, and FIG. 13.

### <6-1. Description 1 of Touch Panel>

In this embodiment, a touch panel 2000 including a display panel and an input device will be described as an example of an electronic device. In addition, an example in which a touch sensor is used as an input device will be described. Note that a light-emitting device of one embodiment of the present invention can be used for a pixel of the display panel.

FIGS. 9A and 9B are perspective views of the touch panel 2000. Note that FIGS. 9A and 9B illustrate only main components of the touch panel 2000 for simplicity.

The touch panel 2000 includes a display panel 2501 and a touch sensor 2595 (see FIG. 9B). The touch panel 2000 also includes a substrate 2510, a substrate 2570, and a substrate 2590. The substrate 2510, the substrate 2570, and the substrate 2590 each have flexibility. Note that one or all of the substrates 2510, 2570, and 2590 may be inflexible.

The display panel 2501 includes a plurality of pixels over the substrate 2510 and a plurality of wirings 2511 through which signals are supplied to the pixels. The plurality of wirings 2511 are led to a peripheral portion of the substrate 2510, and part of the plurality of wirings 2511 form a terminal 2519. The terminal 2519 is electrically connected to an FPC 2509(1).

The substrate 2590 includes the touch sensor 2595 and a plurality of wirings 2598 electrically connected to the touch sensor 2595. The plurality of wirings 2598 are led to a peripheral portion of the substrate 2590, and part of the plurality of wirings 2598 form a terminal. The terminal is electrically connected to an FPC 2509(2). Note that in FIG. 9B, electrodes, wirings, and the like of the touch sensor 2595 provided on the back side of the substrate 2590 (the side facing the substrate 2510) are indicated by solid lines for clarity.

As the touch sensor 2595, a capacitive touch sensor can be used, for example. Examples of the capacitive touch sensor include a surface capacitive touch sensor and a projected capacitive touch sensor.

Examples of the projected capacitive touch sensor include a self-capacitive touch sensor and a mutual capacitive touch sensor, which differ mainly in the driving method. The use of a mutual capacitive touch sensor is preferable because multiple points can be sensed simultaneously.

Note that the touch sensor 2595 illustrated in FIG. 9B is an example of using a projected capacitive touch sensor.

Note that a variety of sensors that can sense proximity or touch of a sensing target such as a finger can be used as the touch sensor 2595.

The projected capacitive touch sensor 2595 includes electrodes 2591 and electrodes 2592. The electrodes 2591 are electrically connected to any of the plurality of wirings 2598, and the electrodes 2592 are electrically connected to any of the other wirings 2598.

The electrodes 2592 each have a shape of a plurality of quadrangles arranged in one direction with one corner of a quadrangle connected to one corner of another quadrangle as illustrated in FIGS. 9A and 9B.

The electrodes 2591 each have a quadrangular shape and are arranged in a direction intersecting with the direction in which the electrodes 2592 extend.

A wiring 2594 electrically connects two electrodes 2591 between which the electrode 2592 is positioned. The intersecting area of the electrode 2592 and the wiring 2594 is preferably as small as possible. Such a structure allows a reduction in the area of a region where the electrodes are not provided, reducing variation in transmittance. As a result, variation in luminance of light passing through the touch sensor 2595 can be reduced.

Note that the shapes of the electrodes 2591 and the electrodes 2592 are not limited thereto and can be any of a variety of shapes. For example, a structure may be employed in which the plurality of electrodes 2591 are arranged so that gaps between the electrodes 2591 are reduced as much as possible, and the electrodes 2592 are spaced apart from the electrodes 2591 with an insulating layer interposed therebetween to have regions not overlapping with the electrodes 2591. In this case, it is preferable to provide, between two adjacent electrodes 2592, a dummy electrode electrically insulated from these electrodes because the area of regions having different transmittances can be reduced.

Note that for example, a transparent conductive film including indium oxide, tin oxide, zinc oxide, or the like (e.g., a film of ITO) can be given as a material of conductive films used for the electrode 2591, the electrode 2592, and the wiring 2598, i.e., wirings and electrodes in the touch panel. Moreover, for example, a low-resistance material is preferably used as the material of the wiring and the electrode in the touch panel. For example, silver, copper, aluminum, a carbon nanotube, graphene, or a metal halide (such as a silver halide) may be used. Alternatively, a metal nanowire including a plurality of conductors with an extremely small width (e.g., a diameter of several nanometers) may be used. Further alternatively, a metal mesh which is a net-like conductor may be used. Examples of such materials include an Ag nanowire, a Cu nanowire, an Al nanowire, an Ag mesh, a Cu mesh, and an Al mesh. For example, in the case of using an Ag nanowire for the wiring and the electrode in the touch panel, a visible light transmittance of 89% or more and a sheet resistance of 40 Ω/cm² or more and 100 Ω/cm² or less can be achieved. A metal nanowire, a metal mesh, a carbon nanotube, graphene, and the like, which are examples of a material that can be used for the above-described wiring and electrode in the touch panel, have a high visible light transmittance; therefore, they may be used for an electrode of a display element (e.g., a pixel electrode or a common electrode).

### <6-2. Display Panel>

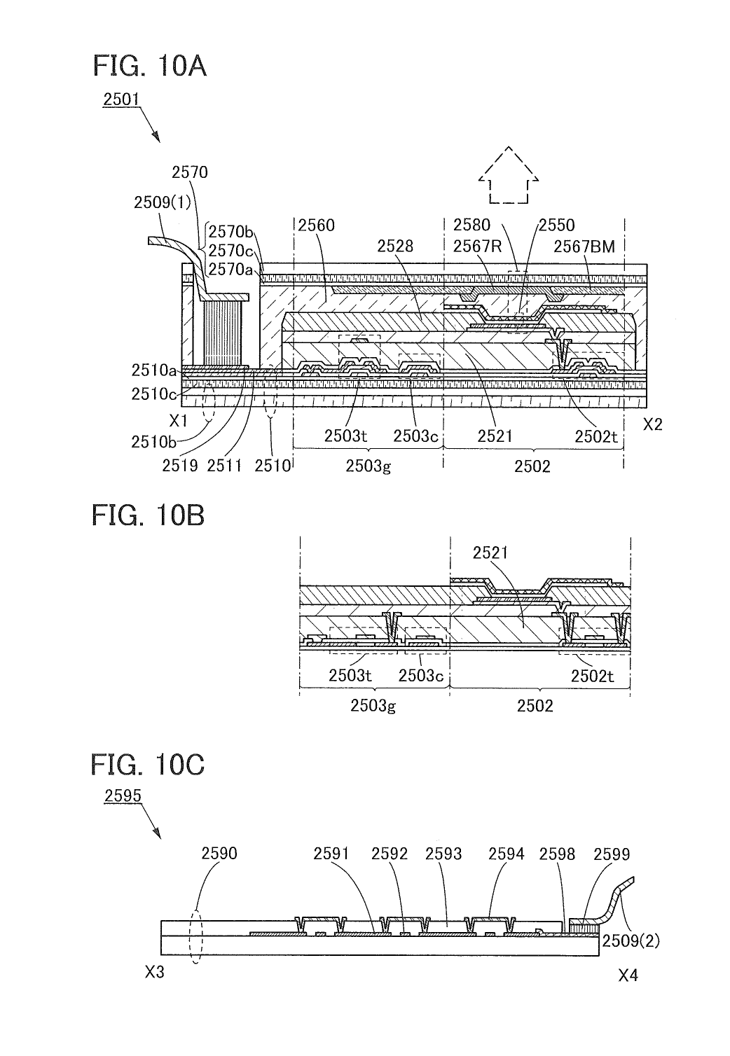

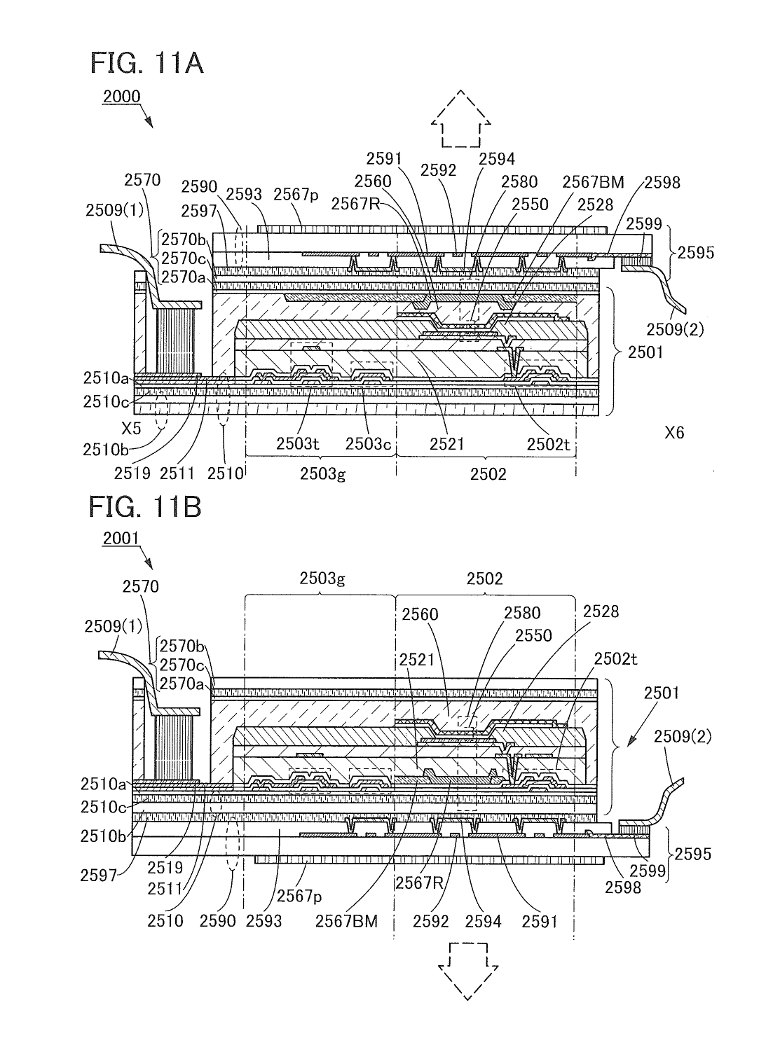

Next, the display panel 2501 will be described in detail with reference to FIG. 10A. FIG. 10A corresponds to a cross-sectional view taken along dashed-dotted line X1-X2 in FIG. 9B.

The display panel 2501 includes a plurality of pixels arranged in a matrix. Each of the pixels includes a display element and a pixel circuit for driving the display element.

For the substrate 2510 and the substrate 2570, for example, a flexible material with a vapor permeability of lower than or equal to 10⁻⁵ g/(m²·day), preferably lower than or equal to 10⁻⁶ g/(m²·day) can be favorably used. Alternatively, materials whose thermal expansion coefficients are substantially equal to each other are preferably used for the substrate 2510 and the substrate 2570. For example, the coefficients of linear expansion of the materials are preferably lower than or equal to 1×10⁻³/K, further preferably lower than or equal to 5×10⁻⁵/K, and still further preferably lower than or equal to 1×10⁻⁵/K.

Note that the substrate 2510 is a stacked body including an insulating layer 2510a for preventing impurity diffusion into the light-emitting element, a flexible substrate 2510b , and an adhesive layer 2510c for attaching the insulating layer 2510a and the flexible substrate 2510b to each other. The substrate 2570 is a stacked body including an insulating layer 2570a for preventing impurity diffusion into the light-emitting element, a flexible substrate 2570b , and an adhesive layer 2570c for attaching the insulating layer 2570a and the flexible substrate 2570b to each other.

For the adhesive layer 2510c and the adhesive layer 2570c , for example, materials that include a polyester, a polyolefin, a polyamide (e.g., a nylon, aramid), a polyimide, a polycarbonate, a polyurethane, an acrylic resin, an epoxy resin, or a resin having a siloxane bond can be used.

A sealing layer 2560 is provided between the substrate 2510 and the substrate 2570. The sealing layer 2560 preferably has a refractive index higher than that of air. In the case where light is extracted to the sealing layer 2560 side as illustrated in FIG. 10A, the sealing layer 2560 can also serve as an optical element.

A sealant may be formed in the peripheral portion of the sealing layer 2560. With the use of the sealant, a light-emitting element 2550 can be provided in a region surrounded by the substrate 2510, the substrate 2570, the sealing layer 2560, and the sealant. Note that an inert gas (such as nitrogen or argon) may be used instead of the sealing layer 2560. A drying agent may be provided in the inert gas so as to adsorb moisture or the like. For example, an epoxy-based resin or a glass frit is preferably used as the sealant. As a material used for the sealant, a material which is impermeable to moisture or oxygen is preferably used.

The display panel 2501 includes a pixel 2502. The pixel 2502 includes a light-emitting module 2580.

The pixel 2502 includes the light-emitting element 2550 and a transistor 2502t that can supply electric power to the light-emitting element 2550. Note that the transistor 2502t functions as part of the pixel circuit. The light-emitting module 2580 includes the light-emitting element 2550 and a coloring layer 2567R.

The light-emitting element 2550 includes a lower electrode, an upper electrode, and an EL layer between the lower electrode and the upper electrode. As the light-emitting element 2550, the light-emitting element described in the above embodiment can be used, for example. Note that although only one light-emitting element 2550 is illustrated in FIG. 10A, it is possible to employ the structure including two or more light-emitting elements.

In the case where the sealing layer 2560 is provided on the light extraction side, the sealing layer 2560 is in contact with the light-emitting element 2550 and the coloring layer 2567R.

The coloring layer 2567R is positioned in a region overlapping with the light-emitting element 2550. Accordingly, part of light emitted from the light-emitting element 2550 passes through the coloring layer 2567R and is emitted to the outside of the light-emitting module 2580 as indicated by an arrow in FIG. 10A.

The display panel 2501 includes a light-blocking layer 2567BM on the light extraction side. The light-blocking layer 2567BM is provided so as to surround the coloring layer 2567R.

The coloring layer 2567R is a coloring layer having a function of transmitting light in a particular wavelength region. For example, a color filter for transmitting light in a red wavelength range, a color filter for transmitting light in a green wavelength range, a color filter for transmitting light in a blue wavelength range, a color filter for transmitting light in a yellow wavelength range, or the like can be used. Each color filter can be formed with any of a variety of materials by a printing method, an inkjet method, an etching method using a photolithography technique, or the like.

An insulating layer 2521 is provided in the display panel 2501. The insulating layer 2521 covers the transistor 2502t . Note that the insulating layer 2521 has a function of covering unevenness caused by the pixel circuit to provide a flat surface. The insulating layer 2521 may have a function of suppressing impurity diffusion. This can prevent the reliability of the transistor 2502t or the like from being lowered by impurity diffusion.

The light-emitting element 2550 is formed over the insulating layer 2521. A partition 2528 is provided so as to overlap with an end portion of the lower electrode of the light-emitting element 2550. Note that a spacer for controlling the distance between the substrate 2510 and the substrate 2570 may be formed over the partition 2528.

A scan line driver circuit 2503g includes a transistor 2503t and a capacitor 2503c. Note that the driver circuit can be formed in the same process and over the same substrate as those of the pixel circuits.

The wirings 2511 through which signals can be supplied are provided over the substrate 2510. The terminal 2519 is provided over the wirings 2511. The FPC 2509(1) is electrically connected to the terminal 2519. The FPC 2509(1) has a function of supplying a video signal, a clock signal, a start signal, a reset signal, or the like. Note that the FPC 2509(1) may be provided with a printed wiring board (PWB).

In the display panel 2501, transistors with any of a variety of structures can be used. FIG. 10A illustrates an example of using bottom-gate transistors; however, the present invention is not limited to this example, and top-gate transistors may be used in the display panel 2501 as illustrated in FIG. 10B.

In addition, there is no particular limitation on the polarity of the transistor 2502t and the transistor 2503t . For these transistors, n-channel and p-channel transistors may be used, or either n-channel transistors or p-channel transistors may be used, for example. Furthermore, there is no particular limitation on the crystallinity of a semiconductor film used for the transistors 2502t and 2503t . For example, an amorphous semiconductor film or a crystalline semiconductor film may be used. Examples of semiconductor materials include Group 13 semiconductors (e.g., a semiconductor including gallium), Group 14 semiconductors (e.g., a semiconductor including silicon), compound semiconductors (including oxide semiconductors), organic semiconductors, and the like. An oxide semiconductor that has an energy gap of 2 eV or more, preferably 2.5 eV or more and further preferably 3 eV or more, is preferably used for one of the transistors 2502t and 2503t or both, so that the off-state current of the transistors can be reduced. Examples of the oxide semiconductors include an In—Ga oxide, an In—M—Zn oxide (M represents Al, Ga, Y, Zr, La, Ce, Sn, or Nd), and the like.

### <6-3. Touch Sensor>

Next, the touch sensor 2595 will be described in detail with reference to FIG. 10C. FIG. 10C corresponds to a cross-sectional view taken along dashed-dotted line X3-X4 in FIG. 9B.

The touch sensor 2595 includes the electrodes 2591 and the electrodes 2592 provided in a staggered arrangement on the substrate 2590, an insulating layer 2593 covering the electrodes 2591 and the electrodes 2592, and the wiring 2594 that electrically connects the adjacent electrodes 2591 to each other.

The electrodes 2591 and the electrodes 2592 are formed using a light-transmitting conductive material. As a light-transmitting conductive material, a conductive oxide such as indium oxide, indium tin oxide, indium zinc oxide, zinc oxide, or zinc oxide to which gallium is added can be used. Note that a film including graphene may be used as well. The film including graphene can be formed, for example, by reducing a film containing graphene oxide. As a reducing method, a method with application of heat or the like can be employed.

The electrodes 2591 and the electrodes 2592 may be formed by, for example, depositing a light-transmitting conductive material on the substrate 2590 by a sputtering method and then removing an unnecessary portion by any of various patterning techniques such as photolithography.

Examples of a material for the insulating layer 2593 are a resin such as an acrylic resin or an epoxy resin, a resin having a siloxane bond, and an inorganic insulating material such as silicon oxide, silicon oxynitride, or aluminum oxide.

Openings reaching the electrodes 2591 are fainted in the insulating layer 2593, and the wiring 2594 electrically connects the adjacent electrodes 2591. A light-transmitting conductive material can be favorably used as the wiring 2594 because the aperture ratio of the touch panel can be increased. Moreover, a material with conductivity higher than the conductivities of the electrodes 2591 and 2592 can be favorably used for the wiring 2594 because electric resistance can be reduced.

One electrode 2592 extends in one direction, and the plurality of electrodes 2592 are provided in the form of stripes. The wiring 2594 intersects with the electrode 2592.

Adjacent electrodes 2591 are provided with one electrode 2592 provided therebetween. The wiring 2594 electrically connects the adjacent electrodes 2591.

Note that the plurality of electrodes 2591 are not necessarily arranged in the direction orthogonal to one electrode 2592 and may be arranged to intersect with one electrode 2592 at an angle of more than 0 degrees and less than 90 degrees.

The wiring 2598 is electrically connected to any of the electrodes 2591 and 2592. Part of the wiring 2598 functions as a terminal. For the wiring 2598, a metal material such as aluminum, gold, platinum, silver, nickel, titanium, tungsten, chromium, molybdenum, iron, cobalt, copper, or palladium or an alloy material containing any of these metal materials can be used.

Note that an insulating layer that covers the insulating layer 2593 and the wiring 2594 may be provided to protect the touch sensor 2595.

A connection layer 2599 electrically connects the wiring 2598 to the FPC 2509(2).

As the connection layer 2599, various anisotropic conductive films (ACF), anisotropic conductive pastes (ACP), or the like can be used.

### <6-4. Description 2 of Touch Panel>

Next, the touch panel 2000 will be described in detail with reference to FIG. 11A. FIG. 11A corresponds to a cross-sectional view taken along dashed-dotted line X5-X6 in FIG. 9A.

In the touch panel 2000 illustrated in FIG. 11A, the display panel 2501 described with reference to FIG. 10A and the touch sensor 2595 described with reference to FIG. 10C are attached to each other.

The touch panel 2000 illustrated in FIG. 11A includes an adhesive layer 2597 and an anti-reflective layer 2567p in addition to the components described with reference to FIGS. 10A and 10C.

The adhesive layer 2597 is provided in contact with the wiring 2594. Note that the adhesive layer 2597 attaches the substrate 2590 to the substrate 2570 so that the touch sensor 2595 overlaps with the display panel 2501. The adhesive layer 2597 preferably has a light-transmitting property. A heat curable resin or an ultraviolet curable resin can be used for the adhesive layer 2597. For example, an acrylic resin, a urethane-based resin, an epoxy-based resin, or a siloxane-based resin can be used.

The anti-reflective layer 2567p is positioned in a region overlapping with pixels. As the anti-reflective layer 2567p , a circularly polarizing plate can be used, for example.

Next, a touch panel having a structure different from that illustrated in FIG. 11A will be described with reference to FIG. 11B.

FIG. 11B is a cross-sectional view of a touch panel 2001. The touch panel 2001 illustrated in FIG. 11B differs from the touch panel 2000 illustrated in FIG. 11A in the position of the touch sensor 2595 relative to the display panel 2501. Different parts are described in detail below, and the above description of the touch panel 2000 is referred to for the other similar parts.

The coloring layer 2567R is positioned in a region overlapping with the light-emitting element 2550. The light-emitting element 2550 illustrated in FIG. 11B emits light to the side where the transistor 2502t is provided. Accordingly, part of light emitted from the light-emitting element 2550 passes through the coloring layer 2567R and is emitted to the outside of the light-emitting module 2580 as indicated by an arrow in FIG. 11B.

The touch sensor 2595 is provided on the substrate 2510 side of the display panel 2501.

The adhesive layer 2597 is provided between the substrate 2510 and the substrate 2590 and attaches the touch sensor 2595 to the display panel 2501.

As illustrated in FIG. 11A or 11B, light may be emitted from the light-emitting element through either or both of the substrates 2510 and 2570.

### <6-5. Method for Driving Touch Panel>

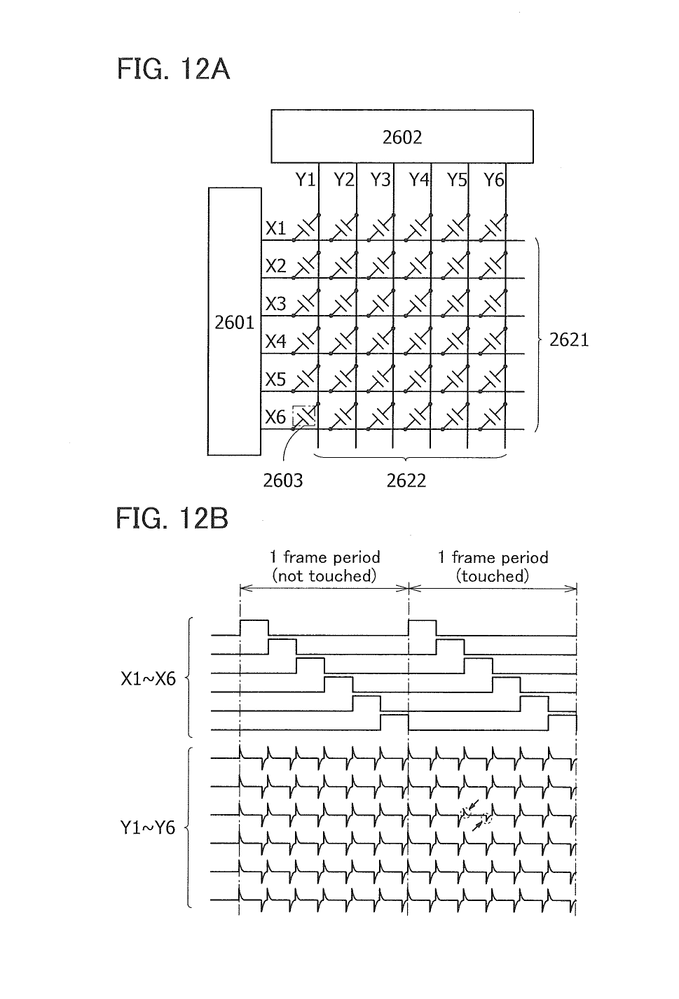

Next, an example of a method for driving a touch panel will be described with reference to FIGS. 12A and 12B.

FIG. 12A is a block diagram illustrating the structure of a mutual capacitive touch sensor. FIG. 12A illustrates a pulse voltage output circuit 2601 and a current sensing circuit 2602. Note that in FIG. 12A, six wirings X1 to X6 represent the electrodes 2621 to which a pulse voltage is applied, and six wirings Y1 to Y6 represent the electrodes 2622 that detect changes in current. FIG. 12A also illustrates capacitors 2603 that are each formed in a region where the electrodes 2621 and 2622 overlap with each other. Note that functional replacement between the electrodes 2621 and 2622 is possible.

The pulse voltage output circuit 2601 is a circuit for sequentially applying a pulse voltage to the wirings X1 to X6. By application of a pulse voltage to the wirings X1 to X6, an electric field is generated between the electrodes 2621 and 2622 of the capacitor 2603. When the electric field between the electrodes is shielded, for example, a change occurs in the capacitor 2603 (mutual capacitance). The approach or contact of a sensing target can be sensed by utilizing this change.

The current sensing circuit 2602 is a circuit for detecting changes in current flowing through the wirings Y1 to Y6 that are caused by the change in mutual capacitance in the capacitor 2603. No change in current value is detected in the wirings Y1 to Y6 when there is no approach or contact of a sensing target, whereas a decrease in current value is detected when mutual capacitance is decreased owing to the approach or contact of a sensing target. Note that an integrator circuit or the like is used for sensing of current values.

FIG. 12B is a timing chart showing input and output waveforms in the mutual capacitive touch sensor illustrated in FIG. 12A. In FIG. 12B, sensing of a sensing target is performed in all the rows and columns in one frame period. FIG. 12B shows a period when a sensing target is not sensed (not touched) and a period when a sensing target is sensed (touched). Sensed current values of the wirings Y1 to Y6 are shown as the waveforms of voltage values.

A pulse voltage is sequentially applied to the wirings X1 to X6, and the waveforms of the wirings Y1 to Y6 change in accordance with the pulse voltage. When there is no approach or contact of a sensing target, the waveforms of the wirings Y1 to Y6 change uniformly in accordance with changes in the voltages of the wirings X1 to X6. The current value is decreased at the point of approach or contact of a sensing target and accordingly the waveform of the voltage value changes.

By detecting a change in mutual capacitance in this manner, the approach or contact of a sensing target can be sensed.

### <6-6. Sensor Circuit>

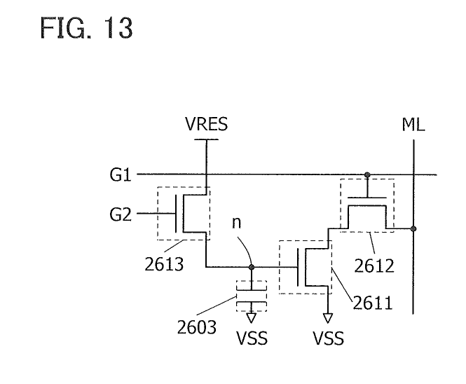

Although FIG. 12A illustrates a passive type touch sensor in which only the capacitor 2603 is provided at the intersection of wirings as a touch sensor, an active type touch sensor including a transistor and a capacitor may be used. FIG. 13 illustrates an example of a sensor circuit included in an active type touch sensor.

The sensor circuit in FIG. 13 includes the capacitor 2603 and transistors 2611, 2612, and 2613.

A signal G2 is input to a gate of the transistor 2613. A voltage VRES is applied to one of a source and a drain of the transistor 2613, and one electrode of the capacitor 2603 and a gate of the transistor 2611 are electrically connected to the other of the source and the drain of the transistor 2613. One of a source and a drain of the transistor 2611 is electrically connected to one of a source and a drain of the transistor 2612, and a voltage VSS is applied to the other of the source and the drain of the transistor 2611. A signal G1 is input to a gate of the transistor 2612, and a wiring ML is electrically connected to the other of the source and the drain of the transistor 2612. The voltage VSS is applied to the other electrode of the capacitor 2603.

Next, the operation of the sensor circuit in FIG. 13 will be described. First, a potential for turning on the transistor 2613 is supplied as the signal G2, and a potential with respect to the voltage VRES is thus applied to a node n connected to the gate of the transistor 2611. Then, a potential for turning off the transistor 2613 is applied as the signal G2, whereby the potential of the node n is maintained.

Then, mutual capacitance of the capacitor 2603 changes owing to the approach or contact of a sensing target such as a finger; accordingly, the potential of the node n is changed from VRES.

In reading operation, a potential for turning on the transistor 2612 is supplied as the signal G1. A current flowing through the transistor 2611, that is, a current flowing through the wiring ML is changed in accordance with the potential of the node n. By sensing this current, the approach or contact of a sensing target can be sensed.

In each of the transistors 2611, 2612, and 2613, an oxide semiconductor layer is preferably used as a semiconductor layer in which a channel region is formed. In particular, such a transistor is preferably used as the transistor 2613 so that the potential of the node n can be held for a long time and the frequency of operation of resupplying VRES to the node n (refresh operation) can be reduced.

The structure described in this embodiment can be used in appropriate combination with any of the structures described in the other embodiments.

### Embodiment 7

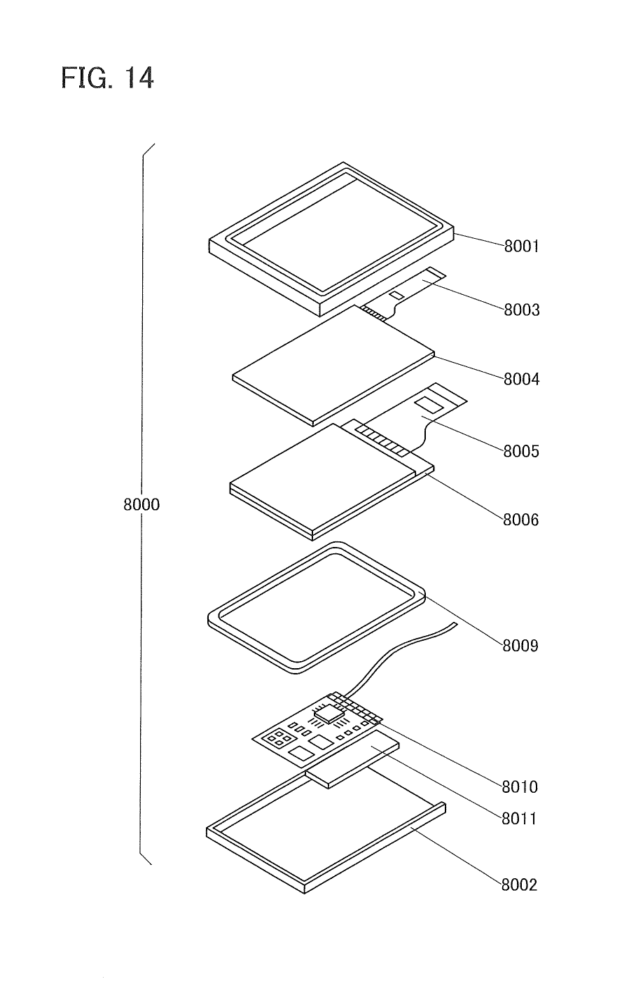

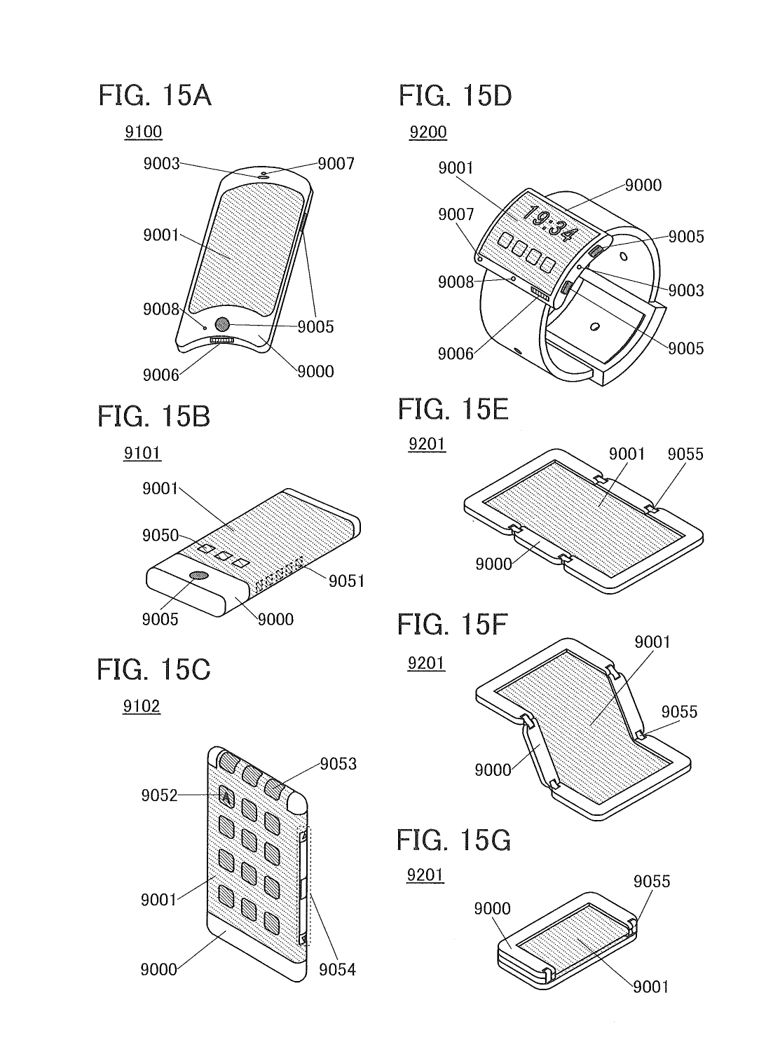

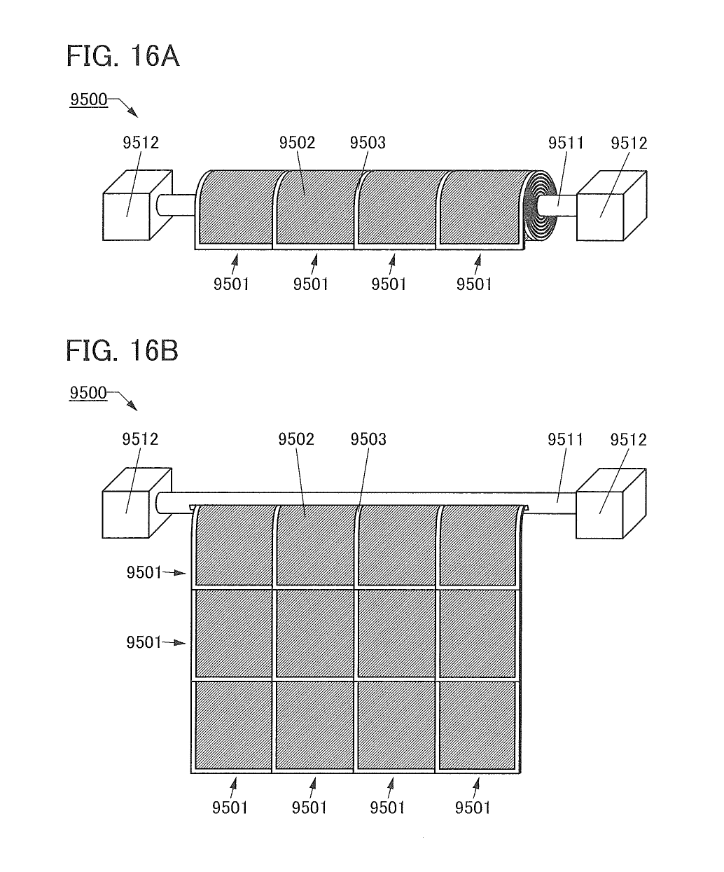

In this embodiment, a display module and electronic devices including a light-emitting element of one embodiment of the present invention will be described with reference to FIG. 14, FIGS. 15A to 15G, and FIGS. 16A and 16B.

### <7-1. Structure Example of Display Module>

In a display module 8000 in FIG. 14, a touch sensor 8004 connected to an FPC 8003, a display panel 8006 connected to an FPC 8005, a frame 8009, a printed circuit board 8010, and a battery 8011 are provided between an upper cover 8001 and a lower cover 8002.

The light-emitting element of one embodiment of the present invention can be used for the display panel 8006, for example.

The shapes and sizes of the upper cover 8001 and the lower cover 8002 can be changed as appropriate in accordance with the sizes of the touch sensor 8004 and the display panel 8006.

The touch sensor 8004 can be a resistive touch panel or a capacitive touch panel and may be formed to overlap with the display panel 8006. A counter substrate (sealing substrate) of the display panel 8006 can have a touch sensor function. A photosensor may be provided in each pixel of the display panel 8006 so that an optical touch sensor is obtained.

The frame 8009 protects the display panel 8006 and also serves as an electromagnetic shield for blocking electromagnetic waves generated by the operation of the printed circuit board 8010. The frame 8009 may serve as a radiator plate.

The printed circuit board 8010 has a power supply circuit and a signal processing circuit for outputting a video signal and a clock signal. As a power source for supplying power to the power supply circuit, an external commercial power source or the battery 8011 provided separately may be used. The battery 8011 can be omitted in the case of using a commercial power source.

The display module 8000 can be additionally provided with a member such as a polarizing plate, a retardation plate, or a prism sheet.

### <7-2. Structure Example of Electronic Device>

FIGS. 15A to 15G illustrate electronic devices. These electronic devices can include a housing 9000, a display portion 9001, a speaker 9003, operation keys 9005, a connection terminal 9006, a sensor 9007, a microphone 9008, and the like.

The electronic devices illustrated in FIGS. 15A to 15G can have a variety of functions, for example, a function of displaying a variety of data (a still image, a moving image, a text image, and the like) on the display portion, a touch sensor function, a function of displaying a calendar, date, time, and the like, a function of controlling a process with a variety of software (programs), a wireless communication function, a function of being connected to a variety of computer networks with a wireless communication function, a function of transmitting and receiving a variety of data with a wireless communication function, a function of reading a program or data stored in a memory medium and displaying the program or data on the display portion, and the like. Note that functions that can be provided for the electronic devices illustrated in FIGS. 15A to 15G are not limited to those described above, and the electronic devices can have a variety of functions. Although not illustrated in FIGS. 15A to 15G, the electronic devices may include a plurality of display portions. The electronic devices may have a camera or the like and a function of taking a still image, a function of taking a moving image, a function of storing the taken image in a memory medium (an external memory medium or a memory medium incorporated in the camera), a function of displaying the taken image on the display portion, or the like.

The electronic devices illustrated in FIGS. 15A to 15G will be described in detail below.

FIG. 15A is a perspective view of a portable information terminal 9100. The display portion 9001 of the portable information terminal 9100 is flexible. Therefore, the display portion 9001 can be incorporated along a bent surface of a bent housing 9000. In addition, the display portion 9001 includes a touch sensor, and operation can be performed by touching the screen with a finger, a stylus, or the like. For example, when an icon displayed on the display portion 9001 is touched, an application can be started.

FIG. 15B is a perspective view of a portable information terminal 9101. The portable information terminal 9101 functions as, for example, one or more of a telephone set, a notebook, and an information browsing system. Specifically, the portable information terminal can be used as a smartphone. Note that the speaker 9003, the connection terminal 9006, the sensor 9007, and the like, which are not illustrated in FIG. 15B, can be positioned in the portable information terminal 9101 as in the portable information terminal 9100 in FIG. 15A. The portable information terminal 9101 can display characters and image information on its plurality of surfaces. For example, three operation buttons 9050 (also referred to as operation icons, or simply, icons) can be displayed on one surface of the display portion 9001. Furthermore, information 9051 indicated by dashed rectangles can be displayed on another surface of the display portion 9001. Examples of the information 9051 include display indicating reception of an incoming email, social networking service (SNS) message, call, and the like; the title and sender of an email and SNS message; the date; the time; remaining battery; and the strength of an antenna. Instead of the information 9051, the operation buttons 9050 or the like may be displayed on the position where the information 9051 is displayed.

FIG. 15C is a perspective view of a portable information terminal 9102. The portable infonnation terminal 9102 has a function of displaying information on three or more surfaces of the display portion 9001. Here, information 9052, information 9053, and information 9054 are displayed on different surfaces. For example, a user of the portable information terminal 9102 can see the display (here, the information 9053) with the portable information terminal 9102 put in a breast pocket of his/her clothes. Specifically, a caller's phone number, name, or the like of an incoming call is displayed in a position that can be seen from above the portable information terminal 9102. Thus, the user can see the display without taking out the portable information terminal 9102 from the pocket and decide whether to answer the call.

FIG. 15D is a perspective view of a watch-type portable information terminal 9200. The portable information terminal 9200 is capable of executing a variety of applications such as mobile phone calls, e-mailing, viewing and editing texts, music reproduction, Internet communication, and computer games. The display surface of the display portion 9001 is bent, and images can be displayed on the bent display surface. The portable information terminal 9200 can employ near field communication conformable to a communication standard. In that case, for example, mutual communication between the portable information terminal 9200 and a headset capable of wireless communication can be performed, and thus hands-free calling is possible. The portable information terminal 9200 includes the connection terminal 9006, and data can be directly transmitted to and received from another information terminal via a connector. Power charging through the connection terminal 9006 is possible. Note that the charging operation may be performed by wireless power feeding without using the connection terminal 9006.

FIGS. 15E, 15F, and 15G are perspective views of a foldable portable information terminal 9201. FIG. 15E is a perspective view illustrating the portable information terminal 9201 that is opened. FIG. 15F is a perspective view illustrating the portable information terminal 9201 that is being opened or being folded. FIG. 15G is a perspective view illustrating the portable information terminal 9201 that is folded. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. By folding the portable information terminal 9201 at a connection portion between two housings 9000 with the hinges 9055, the portable information terminal 9201 can be reversibly changed in shape from an opened state to a folded state. For example, the portable information terminal 9201 can be bent with a radius of curvature of greater than or equal to 1 mm and less than or equal to 150 mm.

FIGS. 16A and 16B are perspective views of a display device including a plurality of display panels. Note that the plurality of display panels are wound in the perspective view in FIG. 16A, and are unwound in the perspective view in FIG. 16B.

A display device 9500 illustrated in FIGS. 16A and 16B includes a plurality of display panels 9501, a hinge 9511, and a bearing 9512. The plurality of display panels 9501 each include a display region 9502 and a light-transmitting region 9503.

Each of the plurality of display panels 9501 is flexible. Two adjacent display panels 9501 are provided so as to partly overlap with each other. For example, the light-transmitting regions 9503 of the two adjacent display panels 9501 can be overlapped each other. A display device having a large screen can be obtained with the plurality of display panels 9501. The display device is highly versatile because the display panels 9501 can be wound depending on its use.

Moreover, although the display regions 9502 of the adjacent display panels 9501 are separated from each other in FIGS. 16A and 16B, without limitation to this structure, the display regions 9502 of the adjacent display panels 9501 may overlap with each other without any space so that a continuous display region 9502 is obtained, for example.

Electronic devices described in this embodiment are characterized by having a display portion for displaying some sort of information. However, a light-emitting device of one embodiment of the present invention can also be used for an electronic device that does not include a display portion. The structure in which the display portion of the electronic device described in this embodiment is flexible and display can be performed on the bent display surface or the structure in which the display portion of the electronic device is foldable is described as an example; however, the structure is not limited thereto and a structure in which the display portion of the electronic device is not flexible and display is performed on a flat portion may be employed.

The structure described in this embodiment can be used in appropriate combination with any of the structures described in the other embodiments.

### Embodiment 8

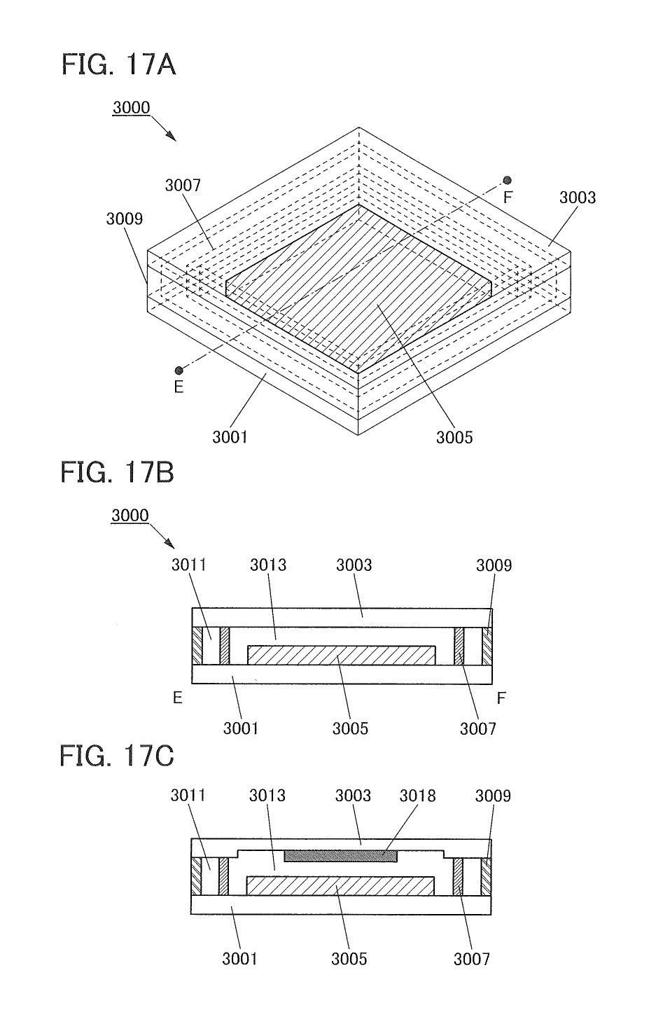

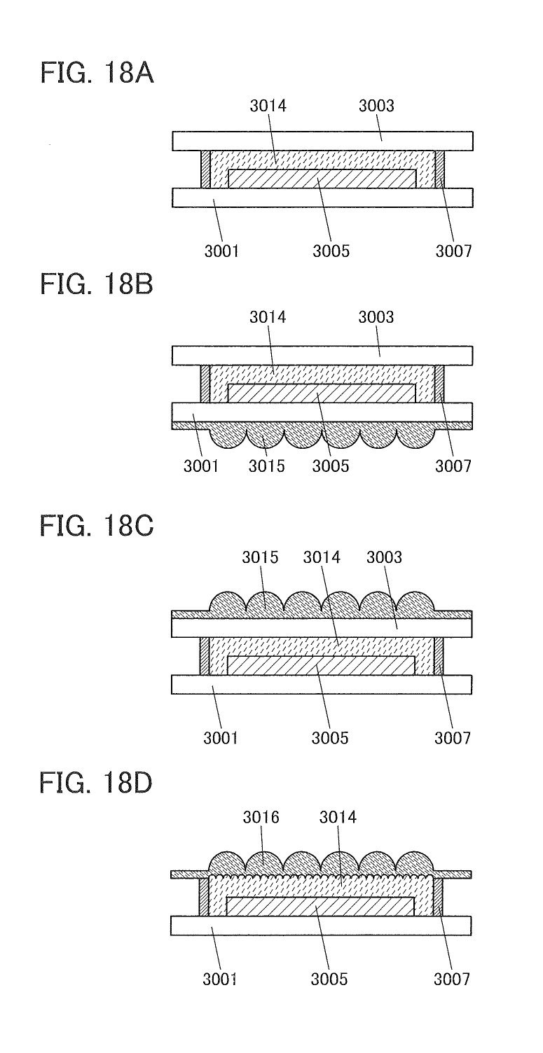

In this embodiment, the light-emitting device of one embodiment of the present invention will be described with reference to FIGS. 17A to 17C and FIGS. 18A to 18D.

### <8. Structure Example of Light-Emitting Device>

FIG. 17A is a perspective view of a light-emitting device 3000 shown in this embodiment, and FIG. 17B is a cross-sectional view taken along dashed-dotted line E-F in FIG. 17A. Note that in FIG. 17A, some components are illustrated by broken lines in order to avoid complexity of the drawing.

The light-emitting device 3000 illustrated in FIGS. 17A and 17B includes a substrate 3001, a light-emitting element 3005 over the substrate 3001, a first sealing region 3007 provided around the light-emitting element 3005, and a second sealing region 3009 provided around the first sealing region 3007.

Light is emitted from the light-emitting element 3005 through one or both of the substrate 3001 and a substrate 3003. In FIGS. 17A and 17B, a structure in which light is emitted from the light-emitting element 3005 to the lower side (the substrate 3001 side) is illustrated.

As illustrated in FIGS. 17A and 17B, the light-emitting device 3000 has a double sealing structure in which the light-emitting element 3005 is surrounded by the first sealing region 3007 and the second sealing region 3009. With the double sealing structure, entry of impurities (e.g., water, oxygen, and the like) from the outside into the light-emitting element 3005 can be favorably suppressed. Note that it is not necessary to provide both the first sealing region 3007 and the second sealing region 3009. For example, only the first sealing region 3007 may be provided.

Note that in FIG. 17B, the first sealing region 3007 and the second sealing region 3009 are each provided in contact with the substrate 3001 and the substrate 3003. However, without limitation to such a structure, for example, one or both of the first sealing region 3007 and the second sealing region 3009 may be provided in contact with an insulating film or a conductive film provided on the substrate 3001. Alternatively, one or both of the first sealing region 3007 and the second sealing region 3009 may be provided in contact with an insulating film or a conductive film provided on the substrate 3003.

The substrate 3001 and the substrate 3003 can have structures similar to the structure of the substrate 200 described in the above embodiment. The light-emitting element 3005 can have a structure similar to that of the light-emitting element described in the above embodiment.

For the first sealing region 3007, a material containing glass (e.g., a glass frit, a glass ribbon, and the like) can be used. For the second sealing region 3009, a material containing a resin can be used. With the use of the material containing glass for the first sealing region 3007, productivity and a sealing property can be improved. Moreover, with the use of the material containing a resin for the second sealing region 3009, impact resistance and heat resistance can be improved. However, the materials used for the first sealing region 3007 and the second sealing region 3009 are not limited to such, and the first sealing region 3007 may be formed using the material containing a resin and the second sealing region 3009 may be formed using the material containing glass.

The glass fit may contain, for example, magnesium oxide, calcium oxide, strontium oxide, barium oxide, cesium oxide, sodium oxide, potassium oxide, boron oxide, vanadium oxide, zinc oxide, tellurium oxide, aluminum oxide, silicon dioxide, lead oxide, tin oxide, phosphorus oxide, ruthenium oxide, rhodium oxide, iron oxide, copper oxide, manganese dioxide, molybdenum oxide, niobium oxide, titanium oxide, tungsten oxide, bismuth oxide, zirconium oxide, lithium oxide, antimony oxide, lead borate glass, tin phosphate glass, vanadate glass, or borosilicate glass. The glass frit preferably contains at least one kind of transition metal to absorb infrared light.

As the above glass frits, for example, a frit paste is applied to a substrate and is subjected to heat treatment, laser light irradiation, or the like. The frit paste contains the glass frit and a resin (also referred to as a binder) diluted by an organic solvent. Note that an absorber which absorbs light having the wavelength of laser light may be added to the glass frit. For example, an Nd:YAG laser or a semiconductor laser is preferably used as the laser. The shape of laser light may be circular or quadrangular.

As the above material containing a resin, for example, materials that include a polyester, a polyolefin, a polyamide (e.g., a nylon, aramid), a polyimide, a polycarbonate, a polyurethane, an acrylic resin, an epoxy resin, or a resin having a siloxane bond can be used.

Note that in the case where the material containing glass is used for one or both of the first sealing region 3007 and the second sealing region 3009, the material containing glass preferably has a thermal expansion coefficient close to that of the substrate 3001. With the above structure, generation of a crack in the material containing glass or the substrate 3001 due to thermal stress can be suppressed.

For example, the following advantageous effect can be obtained in the case where the material containing glass is used for the first sealing region 3007 and the material containing a resin is used for the second sealing region 3009.

The second sealing region 3009 is provided closer to an outer portion of the light-emitting device 3000 than the first sealing region 3007 is. In the light-emitting device 3000, distortion due to external force or the like increases toward the outer portion. Thus, the outer portion of the light-emitting device 3000 where a larger amount of distortion is generated, that is, the second sealing region 3009 is sealed using the material containing a resin and the first sealing region 3007 provided on an inner side of the second sealing region 3009 is sealed using the material containing glass, whereby the light-emitting device 3000 is less likely to be damaged even when distortion due to external force or the like is generated.

Furthermore, as illustrated in FIG. 17B, a first region 3011 corresponds to the region surrounded by the substrate 3001, the substrate 3003, the first sealing region 3007, and the second sealing region 3009. A second region 3013 corresponds to the region surrounded by the substrate 3001, the substrate 3003, the light-emitting element 3005, and the first sealing region 3007.

The first region 3011 and the second region 3013 are preferably filled with, for example, an inert gas such as a rare gas or a nitrogen gas. Note that for the first region 3011 and the second region 3013, a reduced pressure state is preferred to an atmospheric pressure state.

FIG. 17C illustrates a modification example of the structure in FIG. 17B. FIG. 17C is a cross-sectional view illustrating the modification example of the light-emitting device 3000.

FIG. 17C illustrates a structure in which a desiccant 3018 is provided in a recessed portion provided in part of the substrate 3003. The other components are the same as those of the structure illustrated in FIG. 17B.

As the desiccant 3018, a substance which adsorbs moisture and the like by chemical adsorption or a substance which adsorbs moisture and the like by physical adsorption can be used. Examples of the substance that can be used as the desiccant 3018 include alkali metal oxides, alkaline earth metal oxides (e.g., calcium oxide, barium oxide, and the like), sulfate, metal halides, perchlorate, zeolite, silica gel, and the like.

Next, modification examples of the light-emitting device 3000 which is illustrated in FIG. 17B are described with reference to FIGS. 18A to 18D. Note that FIGS. 18A to 18D are cross-sectional views illustrating the modification examples of the light-emitting device 3000 illustrated in FIG. 17B.

In the light-emitting device illustrated in FIG. 18A, the second sealing region 3009 is not provided but only the first sealing region 3007 is provided. Moreover, in the light-emitting device illustrated in FIG. 18A, a region 3014 is provided instead of the second region 3013 illustrated in FIG. 17B.

For the region 3014, for example, materials that include a polyester, a polyolefin, a polyamide (e.g., a nylon or aramid), a polyimide, a polycarbonate, a polyurethane, an acrylic resin, an epoxy resin, or a resin having a siloxane bond can be used.

When the above-described material is used for the region 3014, what is called a solid-sealing light-emitting device can be obtained.

In the light-emitting device illustrated in FIG. 18B, a substrate 3015 is provided on the substrate 3001 side of the light-emitting device illustrated in FIG. 18A.

The substrate 3015 has unevenness as illustrated in FIG. 18B. With a structure in which the substrate 3015 having unevenness is provided on the side through which light emitted from the light-emitting element 3005 is extracted, the efficiency of extraction of light from the light-emitting element 3005 can be improved. Note that instead of the structure having unevenness and illustrated in FIG. 18B, a substrate having a function of a diffusion plate may be provided.

In the light-emitting device illustrated in FIG. 18C, light is extracted through the substrate 3003 side, unlike in the light-emitting device illustrated in FIG. 18A, in which light is extracted through the substrate 3001 side.

The light-emitting device illustrated in FIG. 18C includes the substrate 3015 on the substrate 3003 side. The other components are the same as those of the light-emitting device illustrated in FIG. 18B.

In the light-emitting device illustrated in FIG. 18D, the substrate 3003 and the substrate 3015 included in the light-emitting device illustrated in FIG. 18C are not provided but a substrate 3016 is provided.

The substrate 3016 includes first unevenness positioned closer to the light-emitting element 3005 and second unevenness positioned farther from the light-emitting element 3005. With the structure illustrated in FIG. 18D, the efficiency of extraction of light from the light-emitting element 3005 can be further improved.

Thus, the use of the structure described in this embodiment can provide a light-emitting device in which deterioration of a light-emitting element due to impurities such as moisture and oxygen is suppressed. Alternatively, with the structure described in this embodiment, a light-emitting device having high light extraction efficiency can be obtained.

The structure described in this embodiment can be used in appropriate combination with any of the structures described in the other embodiments.

### Embodiment 9

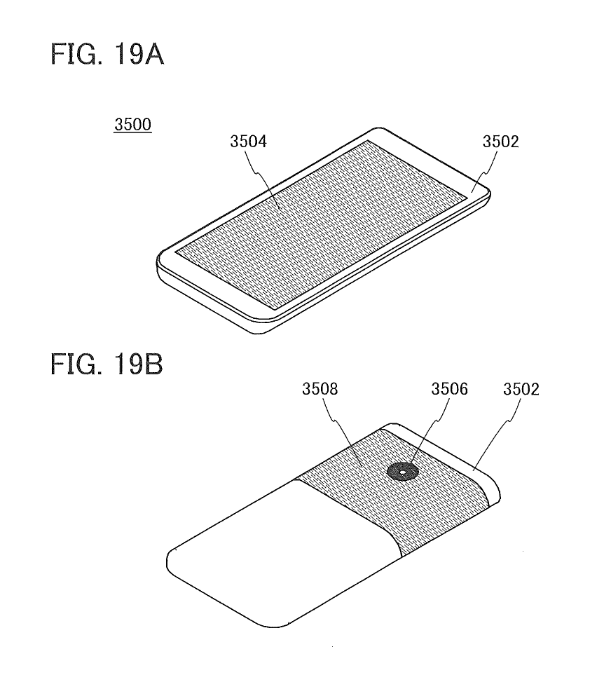

In this embodiment, examples in which the light-emitting device of one embodiment of the present invention is applied to various lighting devices and electronic devices will be described with reference to FIGS. 19A and 19B.

### <9. Structure Example of Lighting Device and Electronic Device>

An electronic device or a lighting device that has a light-emitting region with a curved surface can be obtained with the use of the light-emitting device of one embodiment of the present invention which is manufactured over a substrate having flexibility.

Furthermore, a light-emitting device to which one embodiment of the present invention is applied can also be applied to lighting for motor vehicles, examples of which are lighting for a dashboard, a windshield, a ceiling, and the like.

FIG. 19A is a perspective view illustrating one surface of a multifunction terminal 3500, and FIG. 19B is a perspective view illustrating the other surface of the multifunction terminal 3500. In a housing 3502 of the multifunction terminal 3500, a display portion 3504, a camera 3506, lighting 3508, and the like are incorporated. The light-emitting device of one embodiment of the present invention can be used for the lighting 3508.

The lighting 3508 that includes the light-emitting device of one embodiment of the present invention functions as a planar light source. Thus, unlike a point light source typified by an LED, the lighting 3508 can provide light emission with low directivity. When the lighting 3508 and the camera 3506 are used in combination, for example, imaging can be performed by the camera 3506 with the lighting 3508 lighting or flashing. Because the lighting 3508 functions as a planar light source, a photograph as if taken under natural light can be taken.

Note that the multifunction terminal 3500 illustrated in FIGS. 19A and 19B can have a variety of functions as in the electronic devices illustrated in FIGS. 15A to 15G.

The housing 3502 can include a speaker, a sensor (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone, and the like. When a detection device including a sensor for detecting inclination, such as a gyroscope or an acceleration sensor, is provided inside the multifunction terminal 3500, display on the screen of the display portion 3504 can be automatically switched by determining the orientation of the multifunction terminal 3500 (whether the multifunction terminal is placed horizontally or vertically for a landscape mode or a portrait mode).

The display portion 3504 may function as an image sensor. For example, an image of a palm print, a fingerprint, or the like is taken when the display portion 3504 is touched with the palm or the finger, whereby personal authentication can be performed. Furthermore, by providing a backlight or a sensing light source which emits near-infrared light in the display portion 3504, an image of a finger vein, a palm vein, or the like can be taken. Note that the light-emitting device of one embodiment of the present invention may be used for the display portion 3504.

As described above, lighting devices and electronic devices can be obtained by application of the light-emitting device of one embodiment of the present invention. Note that the light-emitting device can be used for lighting devices and electronic devices in a variety of fields without being limited to the lighting devices and the electronic devices described in this embodiment.

Note that the structure described in this embodiment can be combined as appropriate with any of the structures described in the other embodiments.

### EXAMPLE 1

### 1. SYNTHESIS EXAMPLE 1

In this example, a method for synthesizing 9-[4-(benzo[b]triphenylen-9-yl)phenyl]-9H-carbazole (abbreviation: 9CzPBTp) represented by Structural Formula (100) in Embodiment 1 will be specifically described. The structure of 9CzPBTp is shown below.

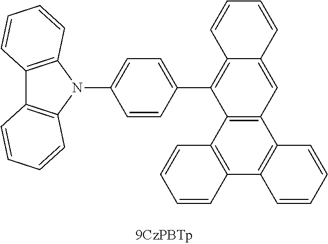 [SMILES: c1ccc2c(-c3ccc(-n4c5ccccc5c5ccccc54)cc3)c3c4ccccc4c4ccccc4c3cc2c1]

A synthesis scheme of 9CzPBTp is described below.

First, 1.5 g (4.2 mmol) of 9-bromobenzo[b]triphenylene, 1.8 g (6.4 mmol) of 4-(9H-carbazol-9-yl)phenylboronic acid, and 1.8 g (13 mmol) of potassium carbonate were put into a 200-mL three-neck flask. To this mixture, 18 mL of toluene, 6 mL of ethanol, and 6 mL of water were added. Then, while the pressure was reduced, this mixture was stirred to be degassed. After that, 49 mg (42 μmol) of tetrakis(triphenylphosphine)palladium(0) was added to this mixture and stirring was performed at 90° C. under a nitrogen stream for 7 hours. After the stirring, an aqueous layer of this mixture was subjected to extraction with toluene.

Then, an organic layer was dried with magnesium sulfate. The resulting mixture was separated by gravity filtration, and the filtrate was concentrated to give a solid. This solid was purified by silica gel column chromatography (as the developing solvent, first, toluene and hexane in a ratio of 1:5 were used, and then toluene and hexane in a ratio of 1:3 were used). The resulting solid was recrystallized with toluene/ethanol. The resulting solid was recrystallized with toluene/ethyl acetate to give 1.3 g of a white solid in a yield of 57%. The above synthesis scheme is shown in (c-1) below.

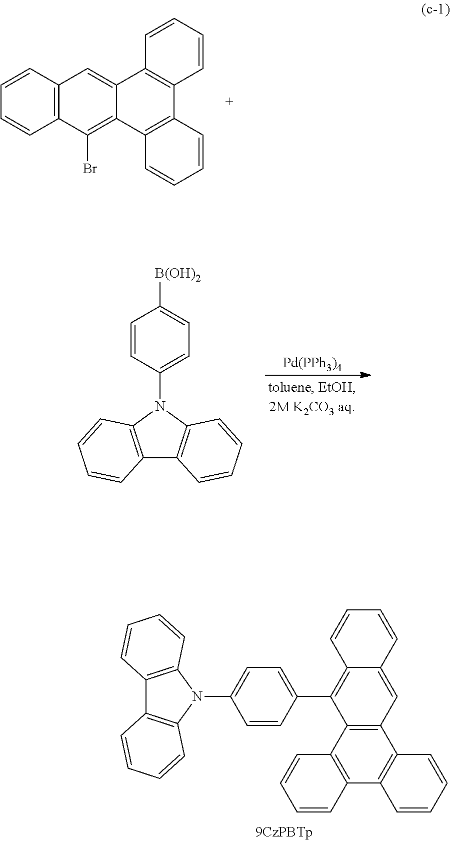 [SMILES: Brc1c2ccccc2cc2c3ccccc3c3ccccc3c12 ; OB(O)c1ccc(-n2c3ccccc3c3ccccc32)cc1 ; c1ccc2c(-c3ccc(-n4c5ccccc5c5ccccc54)cc3)c3c4ccccc4c4ccccc4c3cc2c1 ; "Pd(PPh3)4"]

Next, 1.2 g of the obtained solid was purified by a train sublimation method. The purification by sublimation was conducted by heating at 240° C. under a pressure of 3.0 Pa with a flow rate of an argon gas of 5 mL/min. After the purification by sublimation, 1.1 g of a white solid was obtained at a collection rate of 91%.

This compound was identified as 9CzPBTp, which was the target substance, by nuclear magnetic resonance (¹H NMR).

¹H NMR data of the obtained compound is shown below.

¹H NMR (CDCl₃, 300 MHz): δ=7.14 (t, J1=8.1 Hz, 1H), 7.36 (t, J1=8.1 Hz, 2H), 7.46-7.79 (m, 14H), 7.97 (d, J1=7.8 Hz, 1H), 8.18 (d, J1=8.4 Hz, 1H), 8.21 (d, J1=7.8 Hz, 2H), 8.52 (t, J1=8.1 Hz, 2H), 8.77 (d, J1=7.8 Hz, 1H), 9.19 (s, 1H).

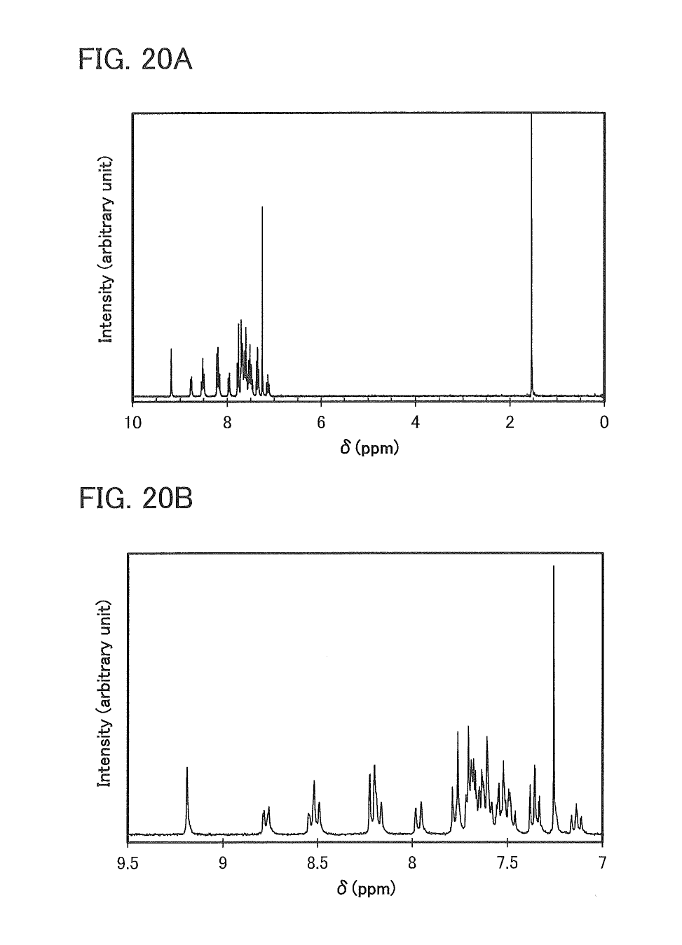

The ¹H NMR charts are shown in FIGS. 20A and 20B. FIG. 20A is the chart showing the range of 0 ppm to 10 ppm, and the FIG. 20B is an enlarged chart showing the range of 7 ppm to 9.5 ppm of FIG. 20A.

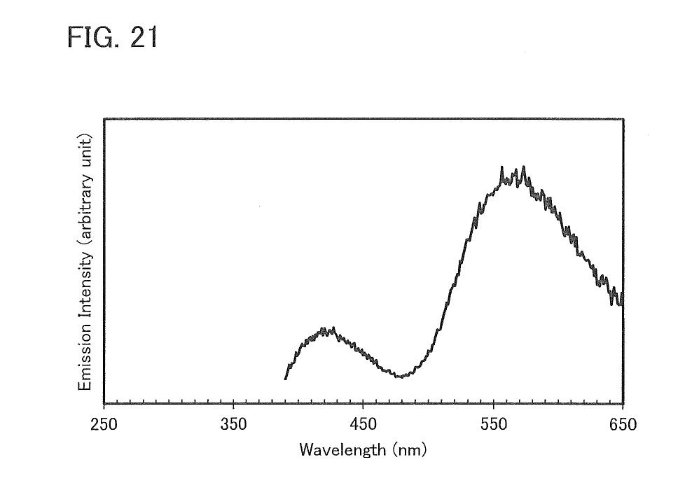

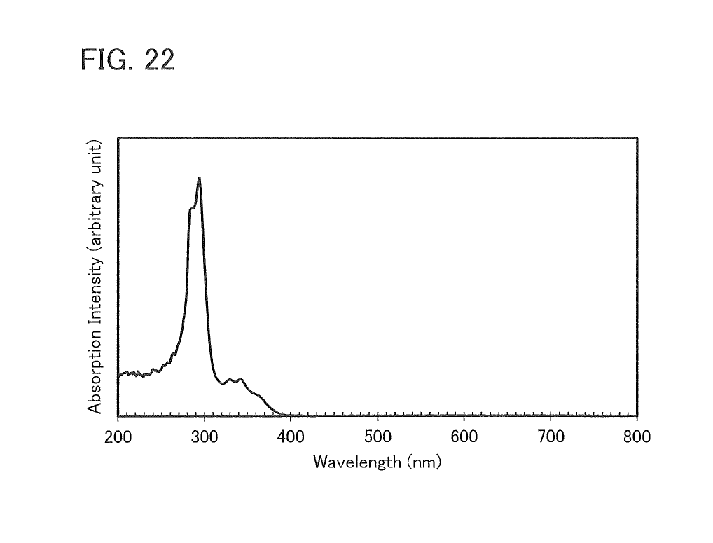

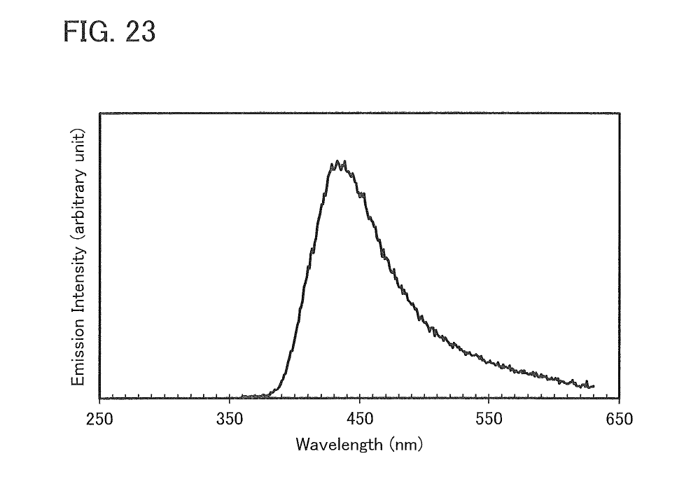

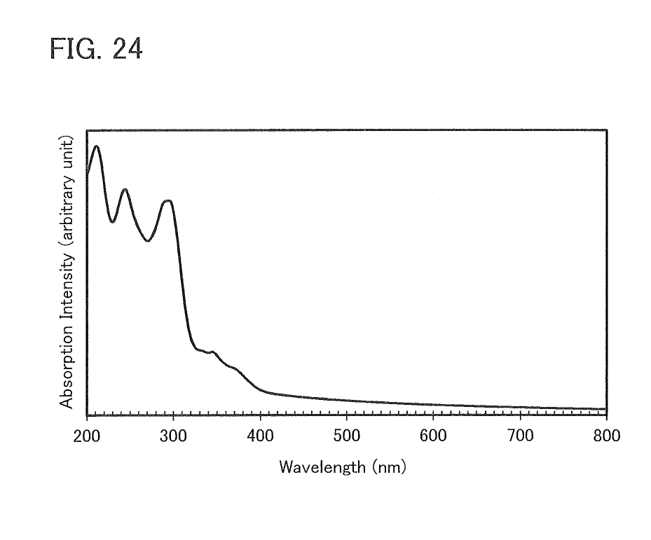

FIG. 21 and FIG. 22 respectively show an emission spectrum and an absorption spectrum of a toluene solution of 9CzPBTp. FIG. 23 and FIG. 24 respectively show an emission spectrum and an absorption spectrum of a thin film of 9CzPBTp. The toluene solution showed emission peaks at 420 nm and 567 nm (excitation wavelength: 342 nm) and absorption peaks at around 330 nm, 342 nm, and 363 nm. The thin film showed an emission peak at 435 nm (excitation wavelength: 340 nm) and absorption peaks at around 211 nm, 244 nm, 261 nm, 288 nm, 295 nm, 333 nm, 345 nm, and 373 nm.

The absorption spectra were measured with an ultraviolet-visible spectrophotometer (V-550, produced by JASCO Corporation). For the measurements of emission spectra and absorption spectra, samples were prepared in such a manner that the solution was put in a quartz cell and the thin film was obtained by deposition onto a quartz substrate. Note that the absorption spectrum of the solution was obtained by subtraction of the absorption spectra of the quartz cell and toluene from those of the quartz cell and the solution, and the absorption spectrum of the thin film was obtained by subtraction of the absorption spectrum of the quartz substrate from the absorption spectra of the thin film on the quartz substrate.

Electrochemical characteristics of a 9CzPBTp solution were also measured.

As a measuring method, cyclic voltammetry (CV) measurement was employed. An electrochemical analyzer (ALS model 600A or 600C, produced by BAS Inc.) was used for the measurement.

The CV measurement revealed that the HOMO level and LUMO level of 9CzPBTp were −5.83 eV and −2.63 eV, respectively.

Note that the cyclic voltammetry (CV) measurement was performed as described below.

As for a solution used for the CV measurement, dehydrated dimethylfonnamide (DMF, manufactured by Sigma-Aldrich Inc., 99.8%, catalog No. 22705-6) was used as a solvent, and tetra-n-butylammonium perchlorate (n-Bu₄NClO₄, manufactured by Tokyo Chemical Industry Co., Ltd., catalog No. T0836), which was a supporting electrolyte, was dissolved in the solvent such that the concentration of tetra-n-butylammonium perchlorate was 100 mmol/L. Further, the object to be measured was dissolved in the solution such that the concentration was 2 mmol/L. A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE-7 reference electrode for nonaqueous solvent, produced by BAS Inc.) was used as a reference electrode. Note that the measurement was conducted at room temperature (20° C. to 25° C.). In addition, the scan rate at the CV measurement was set to 0.1 V/sec.

First, a potential energy (eV) of the reference electrode (Ag/Ag⁺ electrode), which was used in this example, with respect to the vacuum level was calculated. In other words, the Fermi level of the Ag/Ag⁺ electrode was calculated. It is known that the oxidation-reduction potential of ferrocene in methanol is +0.610 [V vs. SHE] with respect to the standard hydrogen electrode (Reference: Christian R. Goldsmith et al., J Am. Chem. Soc., Vol. 124, No. 1, pp. 83-96, 2002).

On the other hand, using the reference electrode used in this example, the oxidation-reduction potential of ferrocene in methanol was calculated to be +0.11 V [vs. Ag/Ag⁺]. Thus, it was found that the potential energy of the reference electrode used in this example was lower than that of the standard hydrogen electrode by 0.50 [eV].

Here, it is known that the potential energy of the standard hydrogen electrode with respect to the vacuum level is −4.44 eV (Reference: Toshihiro Ohnishi and Tamami Koyama, High molecular EL material, Kyoritsu Shuppan, pp. 64-67). Therefore, the potential energy of the reference electrode, which was used in this example, with respect to the vacuum level was calculated as follows: −4.44−0.50=−4.94 [eV].

The oxidation reaction characteristics of the compound of this example were measured in the following manner: the potential of the working electrode with respect to the reference electrode was scanned from approximately 0.2 V to approximately 1.2 V, and then from approximately 1.2 V to approximately 0.2 V.

Now, the calculation of the HOMO level of the object by CV measurement is described in detail. In the measurement of the oxidation reaction characteristics, an oxidation peak potential Eₚₐ [V] and a reduction peak potential E_{pc} [V] were calculated. Therefore, the half-wave potential (intermediate potential between Eₚₐ and E_{pc}) can be calculated as follows: (Eₚₐ+E_{pc})/2 [V]. This shows that the compound of this example can be oxidized by the electric energy of the value of the half-wave potential [V vs. Ag/Ag⁺], and this energy corresponds to the HOMO level.

The reduction reaction characteristics of the compound of this example were measured in the following manner: the potential of the working electrode with respect to the reference electrode was scanned from approximately −1.4 V to approximately −2.5 V, and then from approximately −2.5 V to approximately −1.4 V.

Now, the calculation of the LUMO level of the object by CV measurement is described in detail. In the measurement of the reduction reaction characteristics, a reduction peak potential E_{pc} [V] and an oxidation peak potential Eₚₐ [V] were calculated. Therefore, the half-wave potential (intermediate potential between Eₚₐ and E_{pc}) can be calculated as follows: (Eₚₐ+E_{pc})/2 [V]. This shows that the compound of this example can be reduced by the electric energy of the value of the half-wave potential [V vs. Ag/Ag⁺], and this energy corresponds to the LUMO level.

The structure described in this example can be used in appropriate combination with any of the structures described in the embodiments and the other examples.

### EXAMPLE 2

### 2. SYNTHESIS EXAMPLE 2

In this example, a method for synthesizing 9-[4-(benzo [b]triphenylen-10-yephenyl]-9H-carbazole (abbreviation: 10CzPBTp) represented by Structural Formula (200) in Embodiment 1 will be specifically described. The structure of 10CzPBTp is shown below.

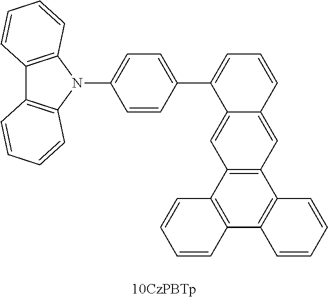 [SMILES: c1cc(-c2ccc(-n3c4ccccc4c4ccccc43)cc2)c2cc3c4ccccc4c4ccccc4c3cc2c1]

A synthesis scheme of 10CzPBTp is described below.

First, 2.0 g (5.6 mmol) of 10-bromobenzo[b]triphenylene, 2.4 g (8.4 mmol) of 4-(9H-carbazol-9-yl)phenylboronic acid, and 2.3 g (17 mmol) of potassium carbonate were put into a 200-mL three-neck flask. To this mixture, 21 mL of toluene, 7 mL of ethanol, and 8 mL of water were added. Then, while the pressure was reduced, this mixture was stirred to be degassed. After that, 65 mg (56 μmol) of tetrakis(triphenylphosphine)palladium(0) was added to this mixture and stirring was performed at 90° C. under a nitrogen stream for 7 hours. After the stirring, the mixture was filtered, the resulting solid was washed with water and ethanol. Then, toluene was added to the resulting solid, and suction filtration through Florisil (registered trademark), Celite (registered trademark), and alumina was conducted to give a filtrate. The obtained filtrate was concentrated to give a solid. This solid was purified by silica gel column chromatography (as the developing solvent, toluene and hexane in a ratio of 1:3 were used). Furthermore, an aqueous layer of the filtrate after the stirring was subjected to extraction with toluene, and an organic layer of the filtrate was dried with magnesium sulfate. The resulting mixture was separated by gravity filtration, and the filtrate was concentrated to give a solid. This solid was purified by silica gel column chromatography (as the developing solvent, toluene and hexane in a ratio of 1:3 were used) to give a solid. The above solids were combined and recrystallized with toluene/ethyl acetate to give 2.0 g of a white solid in a yield of 69%. The above synthesis scheme is shown in (c-2) below.

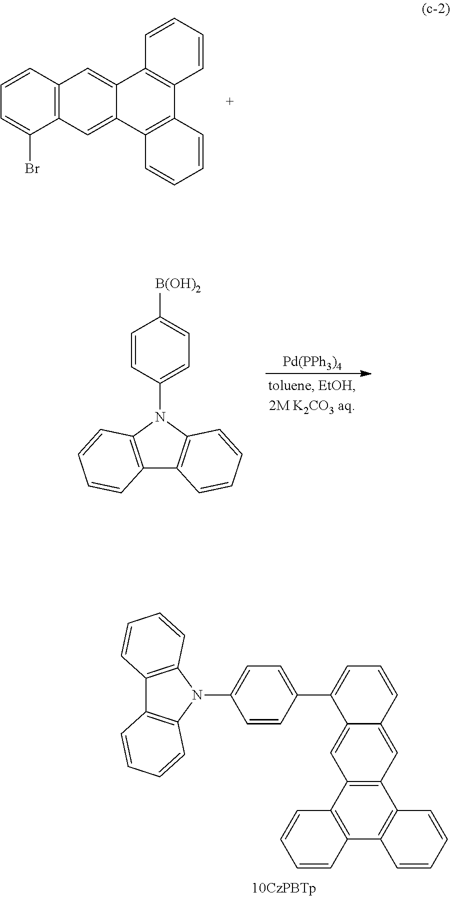 [SMILES: Brc1cccc2cc3c4ccccc4c4ccccc4c3cc12 ; OB(O)c1ccc(-n2c3ccccc3c3ccccc32)cc1 ; c1cc(-c2ccc(-n3c4ccccc4c4ccccc43)cc2)c2cc3c4ccccc4c4ccccc4c3cc2c1 ; "Pd(PPh3)4"]

Next, 1.9 g of the obtained solid was purified by a train sublimation method. The purification by sublimation was conducted by heating at 265° C. under a pressure of 3.0 Pa with a flow rate of an argon gas of 15 mL/min. After the purification by sublimation, 1.1 g of a white solid was obtained at a collection rate of 57%.

This compound was identified as 10CzPBTp, which was the target substance, by nuclear magnetic resonance (¹H NMR).

¹H NMR data of the obtained compound is shown below.

¹H NMR (CDCl₃, 300 MHz): δ=7.36 (t, J1=7.8 Hz, 2H), 7.51 (t, J1=7.2 Hz, 2H), 7.57-7.72 (in, 8H), 7.83 (d, J1=8.4 Hz, 2H), 7.91 (d, J1=8.1 Hz, 2H), 8.17 (d, J1=7.8 Hz, 1H), 8.22 (d, J1=7.8 Hz, 2H), 8.50 (d, J1=6.9 Hz, 1H), 8.57-8.61 (in, 2H), 8.83 (d, J1=7.5 Hz, 1H), 9.19 (s, 1H), 9.32 (s, 1H).

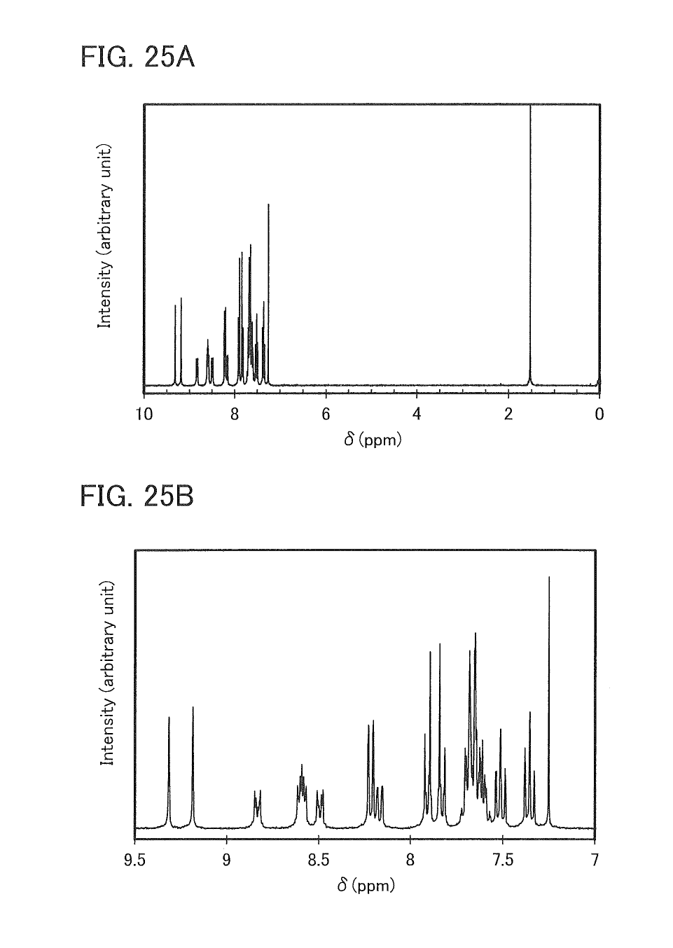

The ¹H NMR charts are shown in FIGS. 25A and 25B. FIG. 25A is the chart showing the range of 0 ppm to 10 ppm, and the FIG. 25B is an enlarged chart showing the range of 7 ppm to 9.5 ppm of FIG. 25A.

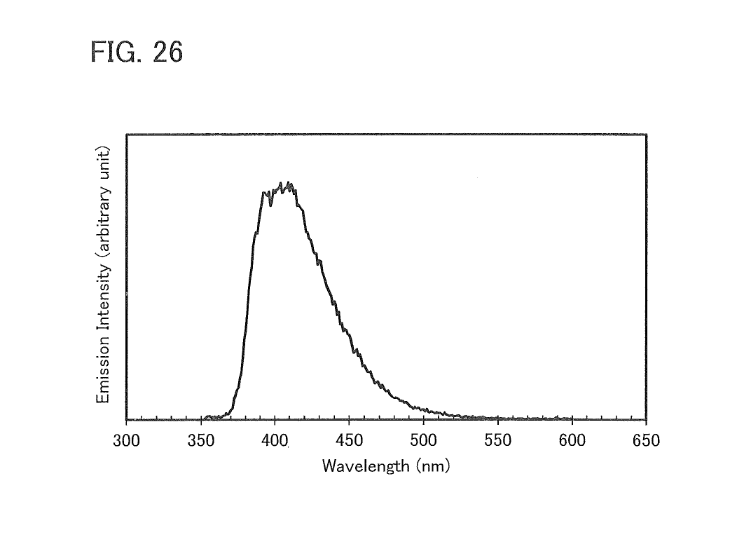

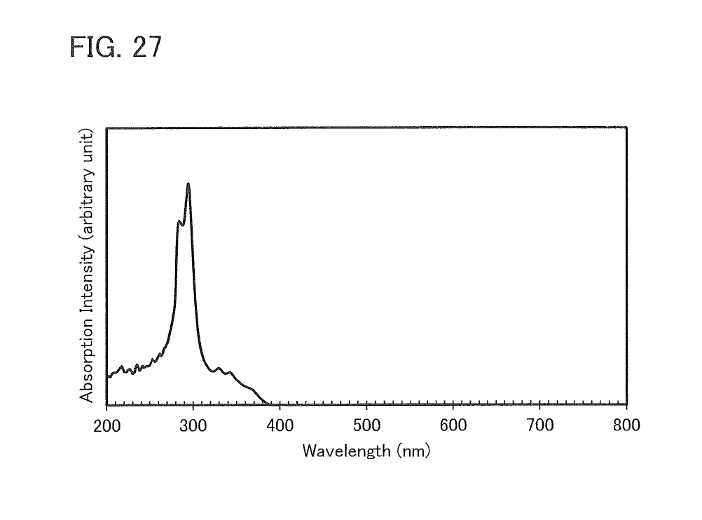

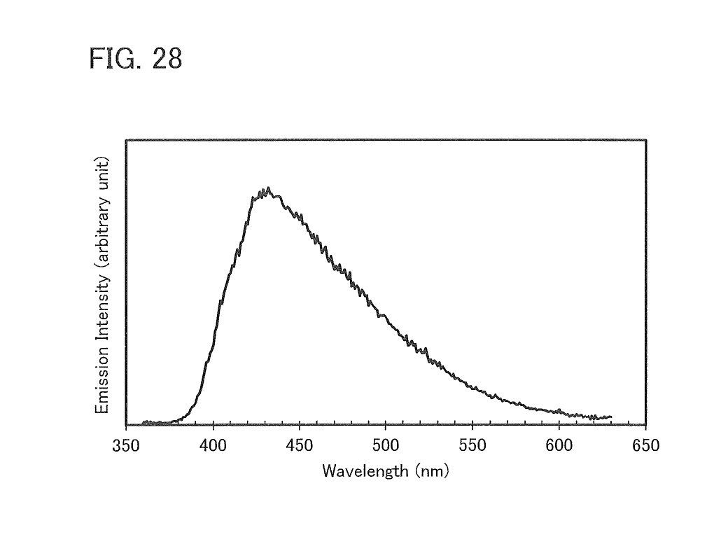

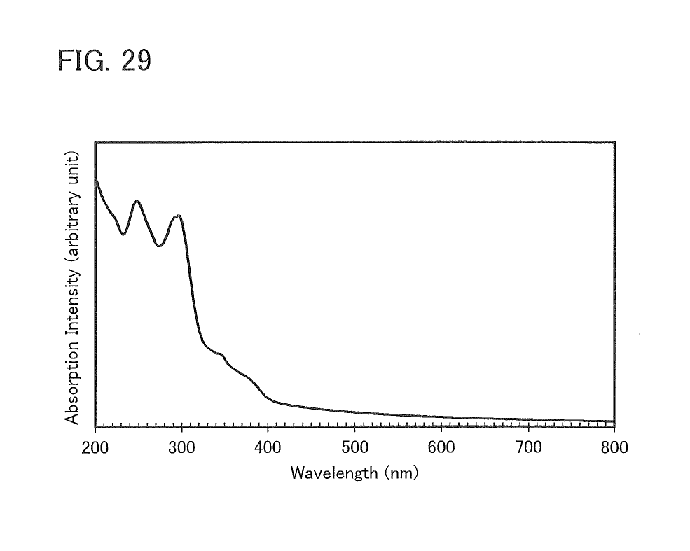

FIG. 26 and FIG. 27 respectively show an emission spectrum and an absorption spectrum of a toluene solution of 10CzPBTp. FIG. 28 and FIG. 29 respectively show an emission spectrum and an absorption spectrum of a thin film of 10CzPBTp. The toluene solution showed emission peaks at 393 nm and 409 nm (excitation wavelength: 343 nm) and absorption peaks at around 284 nm, 295 nm, 330 nm, 343 nm, and 368 nm. The thin film showed an emission peak at 432 nm (excitation wavelength: 340 nm) and absorption peaks at around 248 nm, 264 nm, 290 nm, 296 nm, 333 nm, 346 nm, and 382 nm.

Note that the absorption spectra were measured with the apparatus described in Example 1, and the emission spectra and absorption spectra were measured by the method described in Example 1.

Electrochemical characteristics of a 10CzPBTp solution were also measured.

As a measuring method, cyclic voltammetry (CV) measurement was employed.

The CV measurement revealed that the HOMO level and LUMO level of 10CzPBTp were −5.84 eV and −2.64 eV, respectively.

Note that the above cyclic voltammetry (CV) measurement was conducted in the same manner as in Example 1 except for the scanning range, which is described below.

The oxidation reaction characteristics of the compound of this example were measured in the following manner: the potential of the working electrode with respect to the reference electrode was scanned from approximately 0.3 V to approximately 1.2 V, and then from approximately 1.2 V to approximately 0.3 V.

The reduction reaction characteristics of the compound of this example were measured in the following manner: the potential of the working electrode with respect to the reference electrode was scanned from approximately −1.5 V to approximately −2.5 V, and then from approximately −2.5 V to approximately −1.5 V.

The structure described in this example can be used in appropriate combination with any of the structures described in the embodiments and the other examples.

### EXAMPLE 3

### 3. SYNTHESIS EXAMPLE 3

In this example, a method for synthesizing 7-[4-(benzo[b]triphenylen-10-yl)phenyl]-7H-dibenzo[c,g]carbazole (abbreviation: 10cgDBCzPBTp) that is represented by Structural Formula (153) in Embodiment 1 will be specifically described. The structure of 10cgDBCzPBTp is shown below.

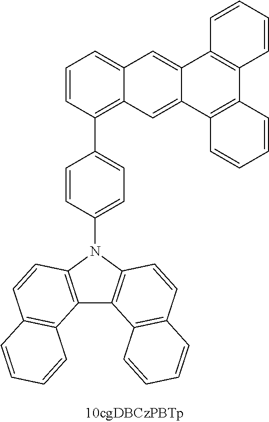 [SMILES: c1cc(-c2ccc(-n3c4ccc5ccccc5c4c4c5ccccc5ccc43)cc2)c2cc3c4ccccc4c4ccccc4c3cc2c1]

A synthesis scheme of 10cgDBCzPBTp is described below.

First, 0.70 g (2.0 mmol) of 10-bromodibenzo[a,c]anthracene, 1.3 g (2.8 mmol) of 7-[4-(4,4,5,5-tetramethyl-1,3,2-dioxaboran-2-yl)phenyl]-7H-dibenzo[c,g]carbazole, and 0.54 g (5.1 mmol) of sodium carbonate were put into a 200-mL three-neck flask. To this mixture, 15 mL of toluene, 5 mL of ethanol, and 5 mL of water were added. Then, while the pressure was reduced, this mixture was stirred to be degassed. After that, 45 mg (39 μmol) of tetrakis(triphenylphosphine)palladium(0) was added to this mixture and stirring was perfoimed at 90° C. under a nitrogen stream for 7 hours. After the stirring, extraction was performed with toluene. An organic layer was dried with magnesium sulfate. The resulting mixture was separated by gravity filtration, and the filtrate was concentrated to give a solid. This solid was purified by silica gel column chromatography (as the developing solvent, toluene and hexane in a ratio of 1:2 were used) to give a solid. The obtained solid was recrystallized with toluene twice to give 0.65 g of a white solid in a yield of 54%. The above synthesis scheme is shown in (c-3) below.

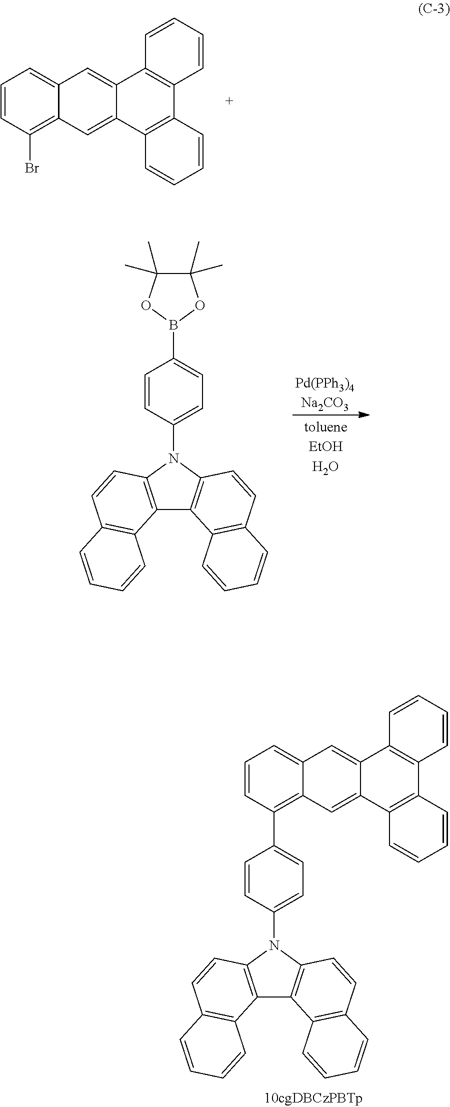 [SMILES: Brc1cccc2cc3c4ccccc4c4ccccc4c3cc12 ; CC1(C)OB(c2ccc(-n3c4ccc5ccccc5c4c4c5ccccc5ccc43)cc2)OC1(C)C ; c1cc(-c2ccc(-n3c4ccc5ccccc5c4c4c5ccccc5ccc43)cc2)c2cc3c4ccccc4c4ccccc4c3cc2c1 ; "Pd(PPh3)4Na2CO3"]

Next, 0.63 g of the obtained solid was purified by a train sublimation method. The purification by sublimation was conducted by heating at 320° C. under a pressure of 3.6 Pa with a flow rate of an argon gas of 15 mL/min. After the purification by sublimation, 0.46 g of a pale yellow solid was obtained at a collection rate of 73%.

This compound was identified as 10cgDBCzPBTp, which was the target substance, by nuclear magnetic resonance (¹H NMR).

¹H NMR data of the obtained compound is shown below.

¹H NMR (CDCl₃, 300 MHz): δ=7.55-7.60 (m, 2H), 7.63-7.78 (m, 8H), 7.82-7.88 (in, 4H), 7.94-8.00 (in, 4H), 8.10 (dd, J1=7.8 Hz, J2=0.9 Hz, 2H), 8.20 (dd, J1=7.8 Hz, J2=1.8 Hz, 1H), 8.52-8.55 (m, 1H), 8.59-8.63 (in, 2H), 8.83-8.87 (in, 1H), 9.22 (s, 1H), 9.31 (d, J1=8.7 Hz, 2H), 9.36 (s, 1H).

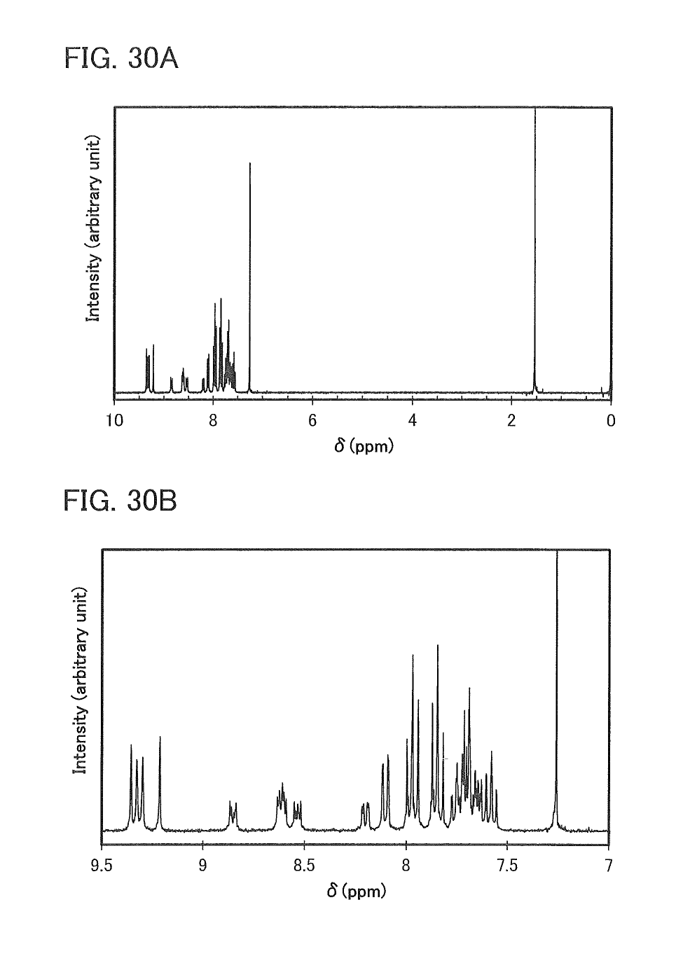

The ¹H NMR charts are shown in FIGS. 30A and 30B. FIG. 30A is the chart showing the range of 0 ppm to 10 ppm, and the FIG. 30B is an enlarged chart showing the range of 7 ppm to 9.5 ppm of FIG. 30A.

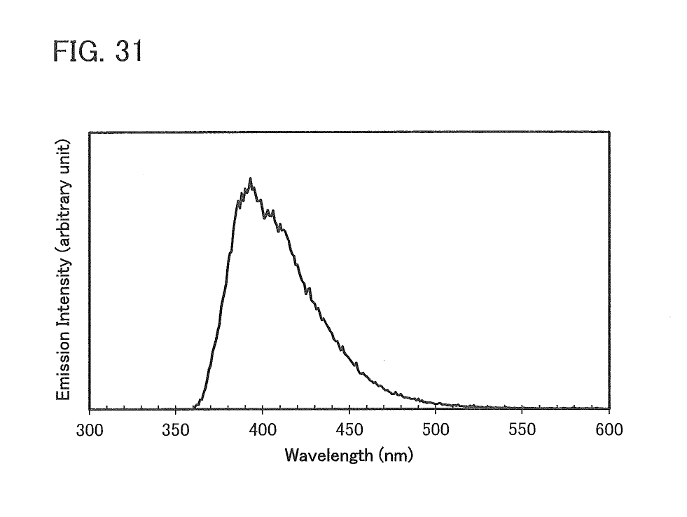

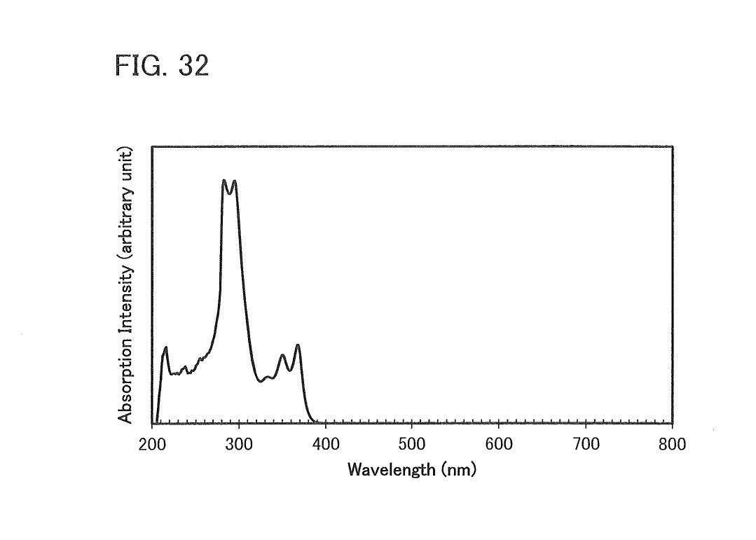

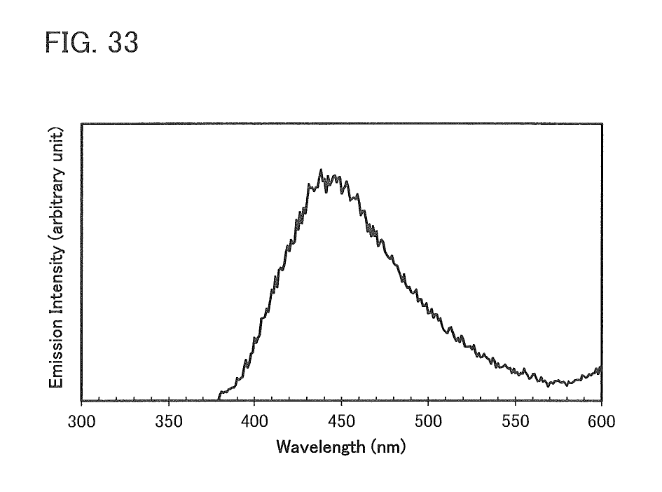

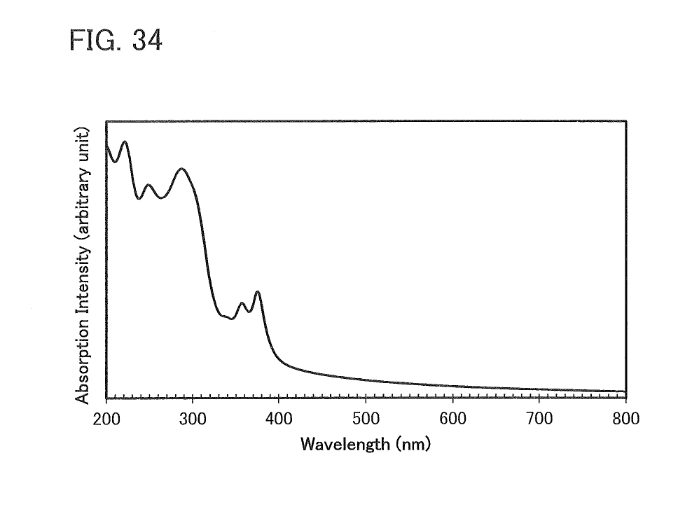

FIG. 31 and FIG. 32 respectively show an emission spectrum and an absorption spectrum of a toluene solution of 1 OcgDBCzPBTp. FIG. 33 and FIG. 34 respectively show an emission spectrum and an absorption spectrum of a thin film of 10cgDBCzPBTp. The toluene solution showed emission peaks at 393 nm and 403 mn (excitation wavelength: 343 nm) and absorption peaks at around 282 mn, 295 nm, 333 nm, 350 nm, and 368 nm. The thin film showed an emission peak at 442 nm (excitation wavelength: 374 mn) and absorption peaks at around 221 nm, 249 nm, 287 mn, 339 nm, 357 nm, and 375 nm

Note that the absorption spectra were measured with the apparatus described in Example 1, and the emission spectra and absorption spectra were measured by the method described in Example 1.

Electrochemical characteristics of a 10cgDBCzPBTp solution were also measured.

As a measuring method, cyclic voltammetry (CV) measurement was employed.

The CV measurement revealed that the HOMO level and LUMO level of 10cgDBCzPBTp were −5.70 eV and −2.63 eV, respectively.

Note that the above cyclic voltammetry (CV) measurement was conducted in the same manner as in Example 1.

The structure described in this example can be used in appropriate combination with any of the structures described in the embodiments and the other examples.

### Example 4

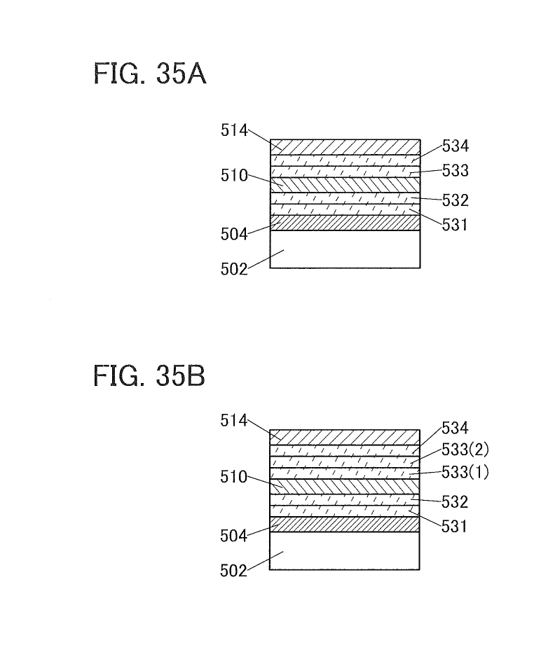

In this example, light-emitting elements of embodiments of the present invention (a light-emitting element 1 and a light-emitting element 2) were fabricated. Structures of the light-emitting elements fabricated in this example will be described with reference to FIG. 35A. Note that FIG. 35A is a cross-sectional view illustrating the structure of each of the light-emitting elements fabricated in this example. Chemical formulae of materials used in this example are shown below.

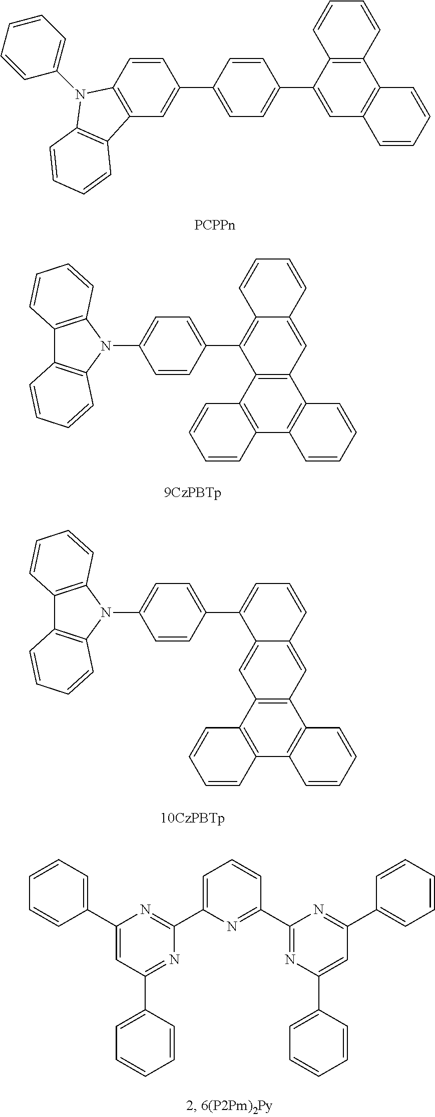 [SMILES: c1ccc(-n2c3ccccc3c3cc(-c4ccc(-c5cc6ccccc6c6ccccc56)cc4)ccc32)cc1 ; c1ccc2c(-c3ccc(-n4c5ccccc5c5ccccc54)cc3)c3c4ccccc4c4ccccc4c3cc2c1 ; c1cc(-c2ccc(-n3c4ccccc4c4ccccc43)cc2)c2cc3c4ccccc4c4ccccc4c3cc2c1 ; c1ccc(-c2cc(-c3ccccc3)nc(-c3cccc(-c4nc(-c5ccccc5)cc(-c5ccccc5)n4)n3)n2)cc1]

### <3-1. Method for Fabricating Light-Emitting Elements 1 and 2>

Next, manufacturing methods of the light-emitting element 1 and the light-emitting element 2 will be described below.

First, a lower electrode 504 was aimed over a substrate 502. For the lower electrode 504, In—Sn—Si oxide (abbreviated to ITSO) was deposited by sputtering to a thickness of 70 nm. Note that the composition of a target used for deposition of the ITSO was In₂O₃:SnO₂:SiO₂=85:10:5 [wt %]. The area of the lower electrode 504 was 4 mm² (2 mm×2 mm). Note that the lower electrode 504 served as an anode of the light-emitting element.

Then, for pretreatment before deposition of an organic compound layer by evaporation, the lower electrode 504 side of the substrate 502 was washed with water, baking was performed at 200° C. for 1 hour, and then UV ozone treatment was performed on a surface of the lower electrode 504 for 370 seconds.

After that, the substrate 502 was transferred into a vacuum evaporation apparatus where the pressure had been reduced to approximately 10⁻⁴ Pa, and was subjected to vacuum baking at 170° C. for 60 minutes in a heating chamber of the vacuum evaporation apparatus, and then the substrate 502 was cooled down for approximately 30 minutes.

Next, the substrate 502 was fixed to a holder provided in the vacuum evaporation apparatus so that a surface of the substrate over which the lower electrode 504 was formed faced downward. In this example, by a resistive heating vacuum evaporation method, a hole-injection layer 531, a hole-transport layer 532, a light-emitting layer 510, an electron-transport layer 533, an electron-injection layer 534, and an upper electrode 514 were sequentially formed. The fabrication method will be described in detail below.

First, the pressure in a vacuum evaporation apparatus was reduced to 10⁻⁴ Pa, followed by formation of the hole-injection layer 531 over the lower electrode 504. For the hole-injection layer 531, 9-[4-(9-phenyl-9H-carbazol-3-yl)phenyl]phenanthrene (abbreviation: PCPPn) and molybdenum oxide were deposited by co-evaporation such that the weight ratio of PCPPn to molybdenum oxide was 2:1. The thickness of the hole-injection layer 531 was 10 nm. Here, co-evaporation is an evaporation method in which different substances are concurrently vaporized from respective evaporation sources.

Then, the hole-transport layer 532 was formed on the hole-injection layer 531. As the hole-transport layer 532, PCPPn was deposited by evaporation. The thickness of the hole-transport layer 532 was 30 nm.

Next, the light-emitting layer 510 was formed over the hole-transport layer 532. Note that the light-emitting element 2 and the light-emitting element 1 were different in the light-emitting layer 510. For the light-emitting layer 510 of the light-emitting element 1, 9CzPBTp synthesized in Example 1 was deposited by evaporation. For the light-emitting layer 510 of the light-emitting element 2, 10CzPBTp synthesized in Example 2 was deposited by evaporation. Note that in each of the light-emitting elements 1 and 2, the thickness of the light-emitting layer 510 was 25 nm.

Next, the electron-transport layer 533 was formed over the light-emitting layer 510. For the electron-transport layer 533, 2,2′-(pyridine-2,6-diyl)bis(4,6-diphenylpyrimidine) (abbreviation: 2,6(P2Pm)2Py) was deposited by evaporation. The thickness of the electron-transport layer 533 was 25 nm. Then, the electron-injection layer 534 was formed over the electron-transport layer 533. For the electron-injection layer 534, lithium fluoride (LiF) was deposited by evaporation. The thickness of the electron-injection layer 534 was 1 mn.

Then, the upper electrode 514 was formed over the electron-injection layer 534. For the upper electrode 514, aluminum (Al) was deposited by evaporation. The thickness of the upper electrode 514 was 200 nm.

Through the above process, the light-emitting elements 1 and 2 were fabricated. Table 1 shows element structures of the light-emitting element 1 and the light-emitting element 2.

TABLE 1

| | | Reference | Thickness | | |
|---|---|---|---|---|---|
| | Layer | numeral | (nm) | Material | Weight ratio |
| Light- | Upper electrode | 514 | 200 | Al | — |
| emitting | Electron-injection | 534 | 1 | LiF | — |
| element 1 | layer | | | | |
| | Electron-transport | 533 | 25 | 2,6(P2Pm)2py | — |
| | layer | | | | |
| | Light-emitting layer | 510 | 25 | 9CzPBTp | — |
| | Hole-transport layer | 532 | 30 | PCPPn | — |
| | Hole-injection layer | 531 | 10 | PCPPn:MoOₓ | 2:1 |
| | Lower electrode | 504 | 70 | ITSO | — |
| Light- | Upper electrode | 514 | 200 | Al | — |
| emitting | Electron-injection | 534 | 1 | LiF | — |
| element 2 | layer | | | | |
| | Electron-transport | 533 | 25 | 2,6(P2Pm)2py | — |
| | layer | | | | |
| | Light-emitting layer | 510 | 25 | 10CzPBTp | — |
| | Hole-transport layer | 532 | 30 | PCPPn | — |
| | Hole-injection layer | 531 | 10 | PCPPn:MoOₓ | 2:1 |
| | Lower electrode | 504 | 70 | ITSO | — |

A sealing substrate 550 was then prepared and sealing was performed by bonding the light-emitting element (the light-emitting element 1 or 2) to the sealing substrate in a glove box in a nitrogen atmosphere such that the light-emitting element was not exposed to the air. Note that for the sealing, a sealant was applied to surround the light-emitting element, irradiation with 365-nm ultraviolet light at 6 J/cm² was performed on the sealant at the time of the sealing, and then, heat treatment was perfoiined at 80° C. for 1 hour.

<3-2. Element characteristics of light-emitting element 1 and light-emitting element 2>

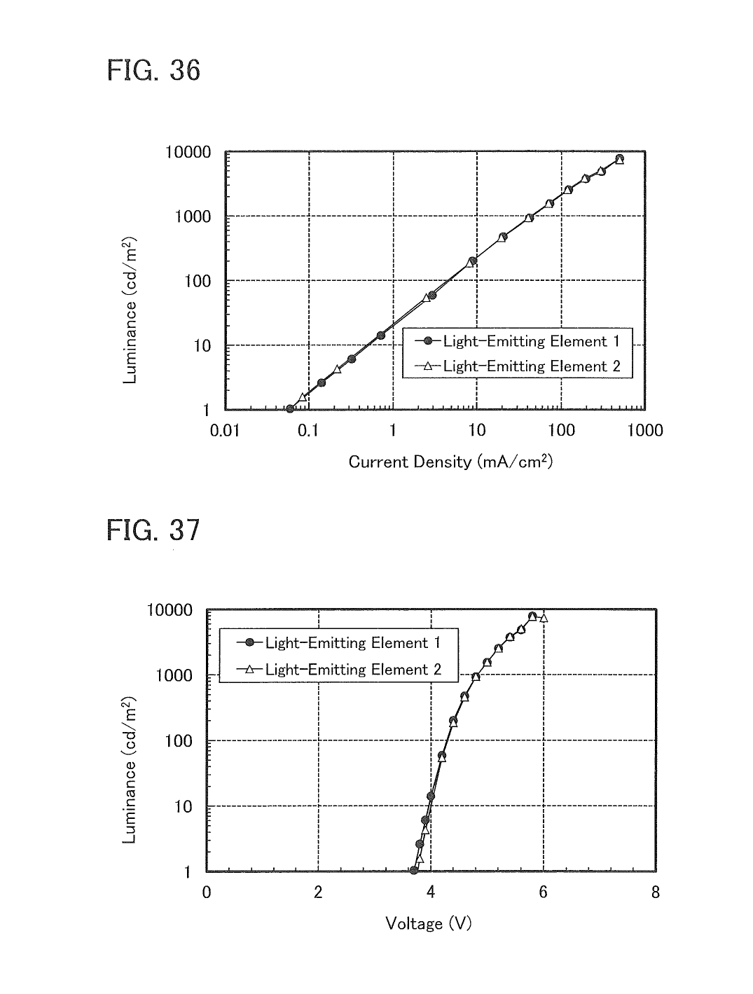

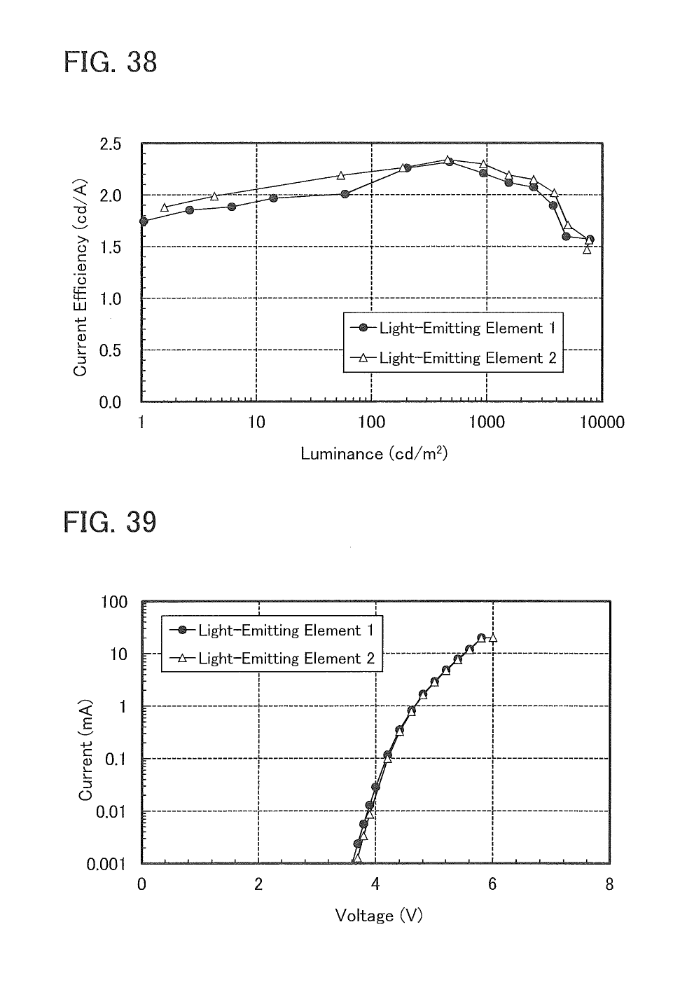

Next, element characteristics of the fabricated light-emitting elements 1 and 2 were measured. FIG. 36 shows the luminance vs. current density characteristics of the light-emitting elements 1 and 2. FIG. 37 shows the luminance vs. voltage characteristics of the light-emitting elements 1 and 2. FIG. 38 shows the current efficiency vs. luminance characteristics of the light-emitting elements 1 and 2. FIG. 39 shows the current vs. voltage characteristics of the light-emitting elements 1 and 2.

Table 2 shows voltage (V), current density (mA/cm²), CIE chromaticity coordinates (x, y), current efficiency (cd/A), and external quantum efficiency (%) of each light-emitting element at a luminance of around 1000 cd/m².

TABLE 2

| | | | | | | | External |
|---|---|---|---|---|---|---|---|
| | | | Current | Chromaticity | | Current | quantum |
| | Voltage | Current | density | coordinates | Luminance | efficiency | efficiency |
| | (V) | (mA) | (mA/cm²) | (x, y) | (cd/m²) | (cd/A) | (%) |
| Light- | 4.8 | 1.69 | 42.2 | (0.15, 0.06) | 932 | 2.2 | 4.1 |
| emitting | | | | | | | |
| element 1 | | | | | | | |
| Light- | 4.8 | 1.62 | 40.6 | (0.14, 0.06) | 932 | 2.3 | 4.6 |
| emitting | | | | | | | |
| element 2 | | | | | | | |

The results shown in FIG. 36 to FIG. 39 and Table 2 show that the light-emitting elements 1 and 2 of embodiments of the present invention have favorable element characteristics.

### <3-3. Measurement of Fluorescence Lifetime of Light-Emitting Elements 1 and 2>

Next, the fluorescence lifetimes of the fabricated light-emitting elements 1 and 2 were measured.

For the light-emitting element 1, blue light emitted from 9CzPBTp was observed. For the light-emitting element 2, blue light emitted from 10CzPBTp was observed.

A picosecond fluorescence lifetime measurement system (manufactured by Hamamatsu Photonics K.K.) was used for the measurement of the fluorescence lifetime. To measure the lifetimes of fluorescence in the light-emitting elements, a square wave pulse voltage was applied to the light-emitting elements, and time-resolved measurements of light, which was attenuated from the falling of the voltage, was performed using a streak camera. The pulse voltage was applied at a frequency of 10 Hz. By integrating data obtained by repeated measurements, data with a high SN ratio was obtained.

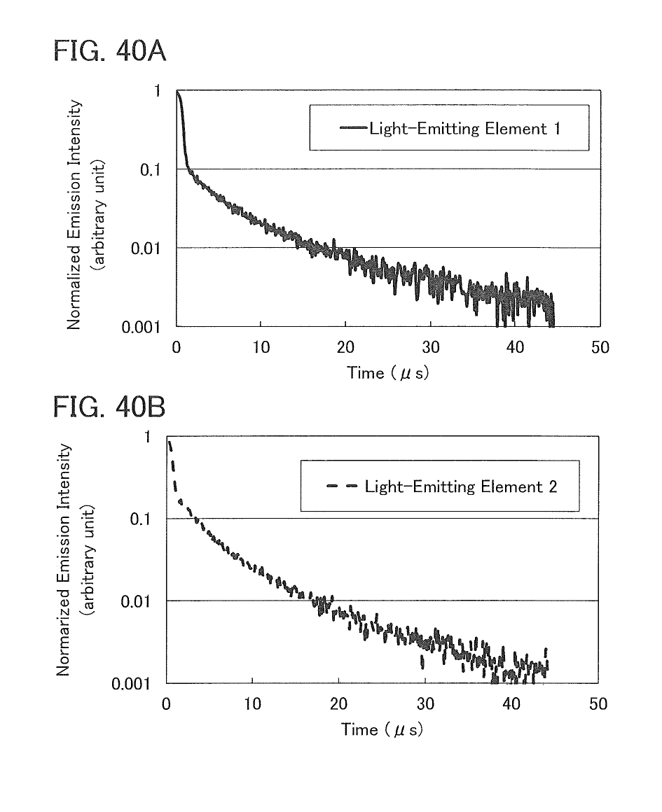

The measurement results of the fluorescence lifetimes of the light-emitting elements 1 and 2 are shown in FIGS. 40A and 40B, respectively. In the fluorescence lifetime measurement, the measurement temperature was room temperature (300 K), a pulse voltage of 4.6 V was applied, a pulse time width was 100 μsec, a negative bias voltage was −5 V, and a measurement time was 50 μs.

In FIGS. 40A and 40B, the vertical axis represents emission intensity normalized to that in a state where carriers are steadily injected (when the pulse voltage is ON), and the horizontal axis represents time elapsed after the falling of the pulse voltage.

The attenuation curves shown in FIGS. 40A and 40B were fitted with an exponential. As a result of the fitting, the fluorescence lifetimes ti of the light-emitting elements 1 and 2 were estimated to be 3.248 μs and 2.558 μs, respectively. Since the lifetime of fluorescence is generally several nanoseconds, light observed from each of the light-emitting elements 1 and 2 is probably fluorescence including delayed fluorescence components.

In the fluorescence lifetime measurement described with reference to FIGS. 40A and 40B, possible causes of the delayed fluorescence other than the formation of a singlet exciton due to triplet-triplet annihilation (TTA) include the formation of a singlet exciton due to recombination of carriers remaining in the light-emitting elements when the pulse voltage is OFF. In these measurements, however, since a negative bias voltage (−5 V) was applied, recombination of the remaining carriers was probably suppressed. Therefore, the delayed fluorescence components shown in the measurement results in FIGS. 40A and 40B were attributed to light emission due to triplet-triplet annihilation (TTA).

Next, the proportion of the delayed fluorescence components in all emissive components was calculated. The proportion of the delayed fluorescence components in each light-emitting element is shown in Table 3.

TABLE 3

| | The proportion of the delayed fluorescence |
|---|---|
| | components in all emissive components (%) |
| Light-emitting | 12.5 |
| element 1 | |
| Light-emitting | 25.2 |
| element 2 | |

The results show that the proportion of the delayed fluorescence components in the light-emitting element 1 was 12.5% and the proportion of the delayed fluorescence components in the light-emitting element 2 was 25.2%.

Accordingly, the benzotriphenylene compound of one embodiment of the present invention has a high proportion of delayed fluorescence components.

The structure described in this example can be used in appropriate combination with any of the structures described in the embodiments and the other examples.

### EXAMPLE 5

In this example, light-emitting elements of embodiments of the present invention (a light-emitting element 3 and a light-emitting element 4) were fabricated. Structures of the light-emitting elements fabricated in this example will be described with reference to FIG. 35B. Note that FIG. 35B is a cross-sectional view illustrating the structure of each of the light-emitting elements fabricated in this example. Chemical formulae of materials used in this example are shown below. For the materials other than those shown below, Example 4 can be referred to.

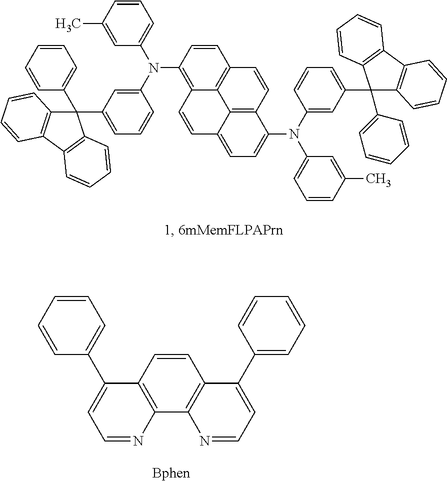 [SMILES: Cc1cccc(N(c2cccc(C3(c4ccccc4)c4ccccc4-c4ccccc43)c2)c2ccc3ccc4c(N(c5cccc(C)c5)c5cccc(C6(c7ccccc7)c7ccccc7-c7ccccc76)c5)ccc5ccc2c3c54)c1 ; c1ccc(-c2ccnc3c2ccc2c(-c4ccccc4)ccnc23)cc1]

### <4-1. Method for Fabricating Light-Emitting Elements 3 and 4>

Next, manufacturing methods of the light-emitting element 3 and the light-emitting element 4 will be described below.

First, the lower electrode 504 was foamed over the substrate 502. As the lower electrode 504, ITSO was deposited by sputtering to a thickness of 70 nm. Note that the composition of a target used for deposition of the ITSO was the same as that in Example 4. The area of the lower electrode 504 was 4 mm² (2 mm×2 mm). Note that the lower electrode 504 serves as an anode of the light-emitting element.

Then, for pretreatment before deposition of an organic compound layer by evaporation, the lower electrode 504 side of the substrate 502 was washed with water, baking was performed at 200° C. for 1 hour, and then UV ozone treatment was performed on a surface of the lower electrode 504 for 370 seconds.

After that, the substrate 502 was transferred into a vacuum evaporation apparatus where the pressure had been reduced to approximately 10⁻⁴ Pa, and was subjected to vacuum baking at 170° C. for 60 minutes in a heating chamber of the vacuum evaporation apparatus, and then the substrate 502 was cooled down for approximately 30 minutes.

Next, the substrate 502 was fixed to a holder provided in the vacuum evaporation apparatus so that a surface of the substrate over which the lower electrode 504 was formed faced downward. In this example, by a resistive heating vacuum evaporation method, the hole-injection layer 531, the hole-transport layer 532, the light-emitting layer 510, an electron-transport layer 533(1), an electron-transport layer 533(2), the electron-injection layer 534, and the upper electrode 514 were sequentially formed. The fabrication method will be described in detail below.

First, the pressure in a vacuum evaporation apparatus was reduced to 10⁻⁴ Pa, followed by formation of the hole-injection layer 531 over the lower electrode 504. As the hole-injection layer 531, PCPPn and molybdenum oxide were deposited by co-evaporation such that the weight ratio of PCPPn to molybdenum oxide was 2:1. The thickness of the hole-injection layer 531 was 10 nm.

Then, the hole-transport layer 532 was formed on the hole-injection layer 531. As the hole-transport layer 532, PCPPn was deposited by evaporation. The thickness of the hole-transport layer 532 was 20 mn.

Next, the light-emitting layer 510 was formed over the hole-transport layer 532. Note that the light-emitting element 3 and the light-emitting element 4 were different in the light-emitting layer 510.

As the light-emitting layer 510 of the light-emitting element 3, 9CzPBTp and N,N-bis(3-methylphenyl)-N,N-bis[3-(9-phenyl-9H-fluoren-9-yl)phenyl]pyrene-1,6-diamine (abbreviation: 1,6mMemFLPAPm) were deposited by co-evaporation such that the weight ratio of 9CzPBTp to 1,6mMemFLPAPrn was 1:0.05. Note that 9CzPBTp served as a host material and 1,6 mMemFLPAPrn served as a fluorescent material (a guest material) in the light-emitting layer 510 of the light-emitting element 3.

As the light-emitting layer 510 of the light-emitting element 4, 10CzPBTp and 1,6mMeinFLPAPrn were deposited by co-evaporation such that the weight ratio of 10CzPBTp to 1,6 mMemFLPAPrn was 1:0.05. Note that 10CzPBTp served as a host material and 1,6mMemFLPAPrn served as a fluorescent material (a guest material) in the light-emitting layer 510 of the light-emitting element 4.

In each of the light-emitting elements 3 and 4, the thickness of the light-emitting layer 510 was 25 nm.

Next, the electron-transport layer 533(1) was formed over the light-emitting layer 510. Note that the light-emitting element 3 and the light-emitting element 4 are different in the electron-transport layer 533(1).

For the electron-transport layer 533(1) of the light-emitting element 3, 9CzPBTp was deposited by evaporation. For the electron-transport layer 533(1) of the light-emitting element 4, 10CzPBTp was deposited by evaporation. In each of the light-emitting elements 3 and 4, the thickness of the electron-transport layer 533(1) was 10 nm.

Then, the electron-transport layer 533(2) was formed over the electron-transport layer 533(1). For the electron-transport layer 533(2), bathophenanthroline (abbreviation: Bphen) was deposited by evaporation. The thickness of the electron-transport layer 533(2) was 15 nm. Then, the electron-injection layer 534 was formed over the electron-transport layer 533(2). For the electron-injection layer 534, LiF was deposited by evaporation. The thickness of the electron-injection layer 534 was 1 nm.

Then, the upper electrode 514 was formed over the electron-injection layer 534. For the upper electrode 514, Al was deposited by evaporation. The thickness of the upper electrode 514 was 200 nm.

Through the above process, the light-emitting elements 3 and 4 were fabricated. Table 4 shows element structures of the light-emitting element 3 and the light-emitting element 4.

TABLE 4

| | | Reference | Thickness | | |
|---|---|---|---|---|---|
| | Layer | numeral | (nm) | Material | Weight ratio |
| Light- | Upper electrode | 514 | 200 | Al | — |
| emitting | Electron- | 534 | 1 | LiF | — |
| element 3 | injection layer | | | | |
| | Electron- | 533(2) | 15 | Bphen | — |
| | transport layer | 533(1) | 10 | 9CzPBTp | — |
| | Light-emitting | 510 | 25 | 9CzPBTp:1,6mMemFLPAPrn | 1:0.05 |
| | layer | | | | |
| | Hole-transport | 532 | 20 | PCPPn | — |
| | layer | | | | |
| | Hole-injection | 531 | 10 | PCPPn:MoOₓ | 2:1 |
| | layer | | | | |
| | Lower electrode | 504 | 70 | ITSO | — |
| Light- | Upper electrode | 514 | 200 | Al | — |
| emitting | Electron- | 534 | 1 | LiF | — |
| element 4 | injection layer | | | | |
| | Electron- | 533(2) | 15 | Bphen | — |
| | transport layer | 533(1) | 10 | 10CzPBTp | — |
| | Light-emitting | 510 | 25 | 10CzPBTp:1,6mMemFLPAPrn | 1:0.05 |
| | layer | | | | |
| | Hole-transport | 532 | 20 | PCPPn | — |
| | layer | | | | |
| | Hole-injection | 531 | 10 | PCPPn:MoOₓ | 2:1 |
| | layer | | | | |
| | Lower electrode | 504 | 70 | ITSO | — |

The sealing substrate 550 was then prepared and sealing was performed by bonding the light-emitting element (the light-emitting element 3 or 4) to the sealing substrate 550 in a glove box in a nitrogen atmosphere such that the light-emitting element was not exposed to the air. Note that for the sealing, a sealant was applied to surround the light-emitting element, irradiation with 365-nm ultraviolet light at 6 J/cm² was performed on the sealant at the time of the sealing, and then, heat treatment was performed at 80° C. for 1 hour.

### <4-2. Element Characteristics of Light-Emitting Element 3 and Light-Emitting Element 4>

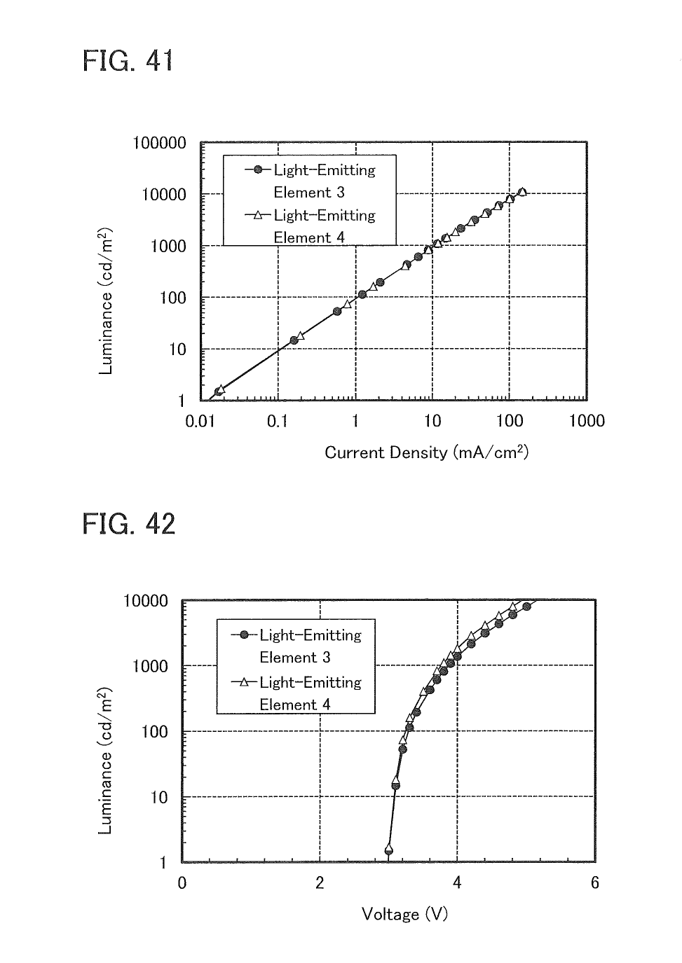

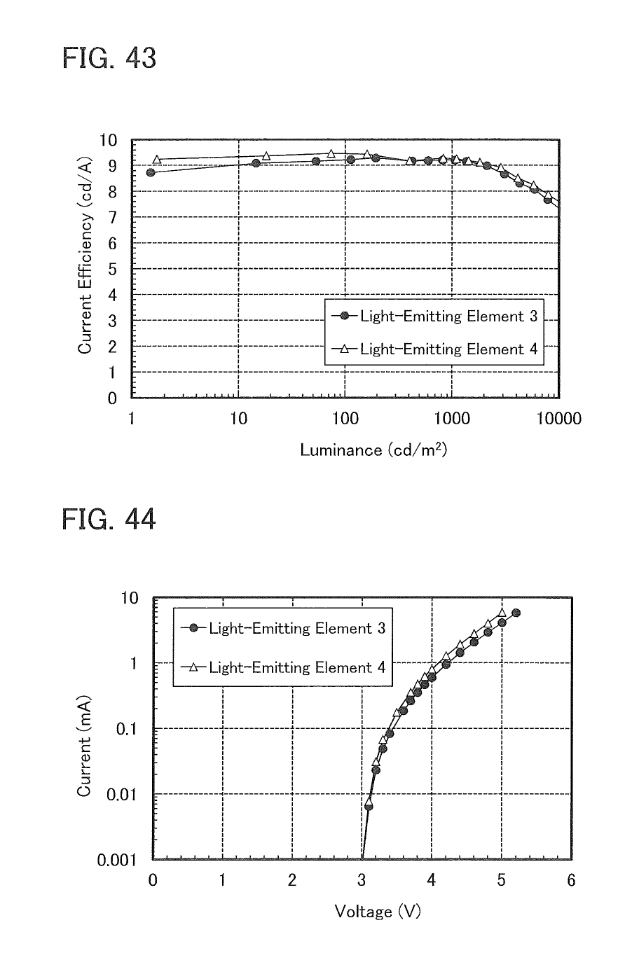

Next, element characteristics of the fabricated light-emitting elements 3 and 4 were measured. FIG. 41 shows the luminance vs. current density characteristics of the light-emitting elements 3 and 4. FIG. 42 shows the luminance vs. voltage characteristics of the light-emitting elements 3 and 4. FIG. 43 shows the current efficiency vs. luminance characteristics of the light-emitting elements 3 and 4. FIG. 44 shows the current vs. voltage characteristics of the light-emitting elements 3 and 4.

Table 5 shows voltage (V), current density (mA/cm²), CIE chromaticity coordinates (x, y), current efficiency (cd/A), and external quantum efficiency (%) of each light-emitting element at a luminance of around 1000 cd/m².

TABLE 5

| | | | | | | | External |
|---|---|---|---|---|---|---|---|
| | | | Current | Chromaticity | | Current | quantum |
| | Voltage | Current | density | coordinates | Luminance | efficiency | efficiency |
| | (V) | (mA) | (mA/cm²) | (x, y) | (cd/m²) | (cd/A) | (%) |
| Light- | 3.9 | 0.46 | 11.6 | (0.14, 0.18) | 1067 | 9.2 | 5.0 |
| emitting | | | | | | | |
| element 3 | | | | | | | |
| Light- | 3.8 | 0.48 | 11.9 | (0.14, 0.18) | 1102 | 9.3 | 5.1 |
| emitting | | | | | | | |
| element 4 | | | | | | | |

### <4-2. Reliability of Light-Emitting Element 3 and Light-Emitting Element 4>

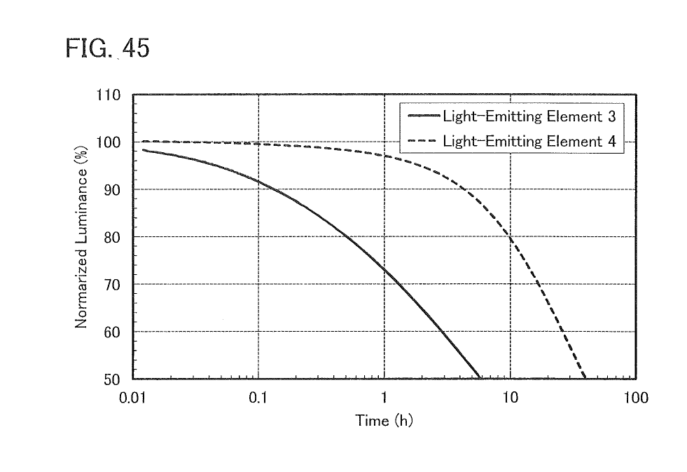

Next, reliability tests were performed on the light-emitting elements 3 and 4. Results of the reliability tests are shown in FIG. 45.

In the reliability tests, the light-emitting elements 3 and 4 were driven under the conditions where the initial luminance was 5000 cd/m² and the current density was constant. In FIG. 45, the vertical axis represents normalized luminance (%) with the initial luminance of 100%, and the horizontal axis represents driving time (h) of the light-emitting element.

As can be observed from the results in FIG. 45, it took approximately 6 hours for the normalized luminance of the light-emitting element 3 to be less than 50%, and it took approximately 40 hours for the normalized luminance of the light-emitting element 4 to be less than 50%.

The structure described in this example can be used in appropriate combination with any of the structures described in the embodiments and the other examples.

This application is based on Japanese Patent Application serial no. 2015-171076 filed with Japan Patent Office on Aug. 31, 2015, the entire contents of which are hereby incorporated by reference.

What is claimed is:

1. A compound represented by General Formula (G1-1): 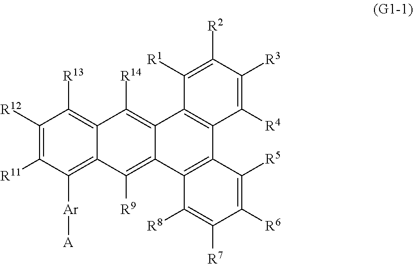
wherein A represents a condensed ring,
wherein each of R¹ to R⁹ and R¹¹ to R¹⁴ independently represents any of hydrogen, an alkyl group having 1 to 6 carbon atoms, a cycloalkyl group having 3 to 6 carbon atoms, and a substituted or unsubstituted aryl group having 6 to 13 carbon atoms, and
wherein Ar represents an arylene group having 6 to 13 carbon atoms.

2. The compound according to claim 1, wherein the condensed ring is one selected from the group consisting of a substituted or unsubstituted carbazolyl group, a substituted or unsubstituted naphthocarbazolyl group, a substituted or unsubstituted dibenzocarbazolyl group, and a substituted or unsubstituted benzocarbazolyl group.

3. The compound according to claim 1,
wherein the condensed ring is a substituted or unsubstituted carbazolyl group, and
wherein any one of a 2-position, a 3-position, and a 9-position of the carbazolyl group is bonded to Ar.

4. The compound according to claim 1, represented by General Formula (G1-2): 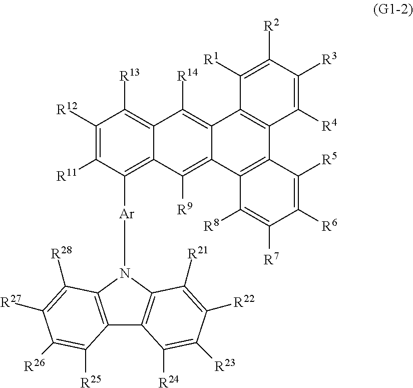
wherein each of R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms.

5. The compound according to claim 1,
wherein the arylene group comprises one or more substituents.

6. The compound according to claim 5, wherein the substituents are bonded to each other to form a ring.

7. The compound according to claim 1, wherein Ar represents a substituted or unsubstituted phenylene group or a substituted or unsubstituted biphenyldiyl group.

8. The compound according to claim 1, wherein Ar represents a substituted or unsubstituted m-phenylene group.

9. The compound according to claim 1, represented by Structural Formula (100):

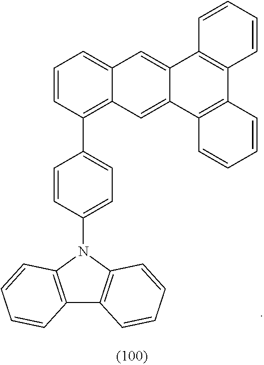 [SMILES: c1cc(-c2ccc(-n3c4ccccc4c4ccccc43)cc2)c2cc3c4ccccc4c4ccccc4c3cc2c1]

10. A light-emitting element comprising:
a first electrode;
a second electrode; and
an EL layer between the first electrode and the second electrode, the EL layer comprising the compound according to claim 1.

11. The light-emitting element according to claim 10, wherein the EL layer comprises a fluorescent material.

12. The light-emitting element according to claim 11, wherein the fluorescent material is capable of emitting blue light.

13. The light-emitting element according to claim 11, wherein the fluorescent material is capable of exhibiting delayed fluorescence.

14. A compound represented by General Formula (G2-1): 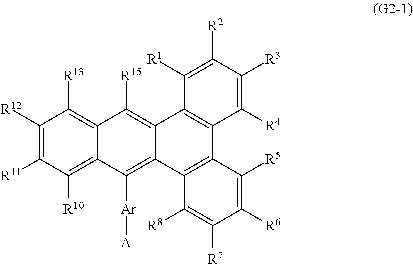
wherein A represents a condensed ring,
wherein each of R¹ to R⁸ and R¹⁰ to R¹³ independently represents any of hydrogen, an alkyl group having 1 to 6 carbon atoms, a cycloalkyl group having 3 to 6 carbon atoms, and a substituted or unsubstituted aryl group having 6 to 13 carbon atoms,
wherein R¹⁵ represents any of hydrogen, an alkyl group having 1 to 6 carbon atoms, and a cycloalkyl group having 3 to 6 carbon atoms, and
wherein Ar represents an arylene group having 6 to 13 carbon atoms.

15. The compound according to claim 14, wherein the condensed ring is one selected from the group consisting of a substituted or unsubstituted carbazolyl group, a substituted or unsubstituted naphthocarbazolyl group, a substituted or unsubstituted dibenzocarbazolyl group, and a substituted or unsubstituted benzocarbazolyl group.

16. The compound according to claim 14,
wherein the condensed ring is a substituted or unsubstituted carbazolyl group, and
wherein any one of a 2-position, a 3-position, and a 9-position of the carbazolyl group is bonded to Ar.

17. The compound according to claim 14, represented by General Formula (G2-2): 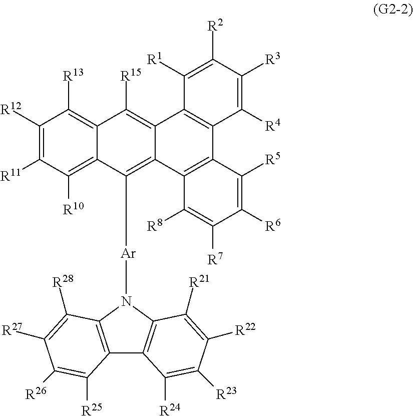
wherein each of R²¹ to R²⁸ independently represents any of hydrogen, an alkyl group having 1 to 6 carbon atoms, a cycloalkyl group having 3 to 6 carbon atoms, and a substituted or unsubstituted aryl group having 6 to 13 carbon atoms.

18. The compound according to claim 14,
wherein the arylene group comprises one or more substituents.

19. The compound according to claim 18, wherein the substituents are bonded to each other to form a ring.

20. The compound according to claim 14, wherein Ar represents a substituted or unsubstituted phenylene group or a substituted or unsubstituted biphenyldiyl group.

21. The compound according to claim 14, wherein Ar represents a substituted or unsubstituted in-phenylene group.

22. The compound according to claim 14, represented by Structural Formula (200):

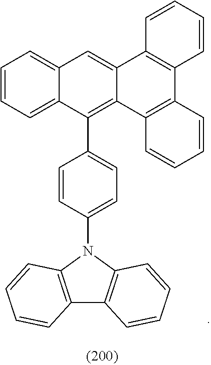 [SMILES: c1ccc2c(-c3ccc(-n4c5ccccc5c5ccccc54)cc3)c3c4ccccc4c4ccccc4c3cc2c1]

23. A light-emitting element comprising:
a first electrode;
a second electrode; and
an EL layer between the first electrode and the second electrode, the EL layer comprising the compound according to claim 14.

24. The light-emitting element according to claim 23, wherein the EL layer comprises a fluorescent material.

25. The light-emitting element according to claim 24, wherein the fluorescent material is capable of emitting blue light.

26. The light-emitting element according to claim 24, wherein the fluorescent material is capable of exhibiting delayed fluorescence.
